(19) United States
(12) Patent Application Publication
(10) Pub. No.: US 2008/0221078 A1
(43) Pub. Date: Sep. 11, 2008

(54) Use of a CB1 Antagonist for Treating Side Effects and Negative Symptoms of Schizophrenia

(75) Inventors: Mark BLACK, Lebanon, NJ (US); Beth BOROWSKY, Flemington, NJ (US); Nancy ROGACKI, South Plainfield, NJ (US); Yaw SENYAH, Princeton, NJ (US); Rachel STEVENS, Lakewood, NJ (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 12/125,285

(22) Filed: May 22, 2008

Related U.S. Application Data
(63) Continuation of application No. PCT/US2006/046547, filed on Dec. 7, 2006.
(60) Provisional application No. 60/748,434, filed on Dec. 8, 2005.

(51) Int. Cl. A61K 31/397; C07D 401/04; C07D 239/24; C07D 215/36; C07D 205/04; A61P 25/18

(52) U.S. Cl. 514/210.21; 548/952; 546/172; 544/298; 546/268.1; 514/210.01; 514/210.2

(57) ABSTRACT

The present invention discloses and claims a method of treating cognition deficits in a patient suffering from schizophrenia by administering to said patient a therapeutically effective amount of a CB1 receptor antagonist as described herein. In another aspect, this invention also discloses and claims a combination of one or more CB1 receptor antagonists and of one or more antipsychotic agents useful in the treatment of psychiatric disorders. The combination of this invention provides synergistic results in that the combination improves positive and negative symptoms of schizophrenia, weight gain and catalepsy.

### CROSS REFERENCE TO RELATED APPLICATIONS

This application is a continuation of International application No. PCT/US2006/046,547, filed Dec. 7, 2006, which is incorporated herein by reference in its entirety; which claims the benefit of U.S. Provisional Application No. 60/748,434, filed Dec. 8, 2005.

### BACKGROUND OF THE INVENTION

1. Field of the Invention

The present invention relates to the use of one or more cannabinoid 1 receptor antagonists (CB1 receptor antagonist) to treat the side effects and negative symptoms of schizophrenia. More specifically, the present invention relates to use of at least one CB 1 antagonist optionally in combination with one or more antipsychotic agents to improve working memory and negative symptoms of schizophrenia and to reverse antipsychotic-induced catalepsy.

2. Description of the Art

CB1 receptor antagonists have been developed for the treatment of schizophrenia (D. Kendall, Curr. Opin. Cent. Peripher. Nerv. Syst. Invest. Drugs, 2(1), 112-122, 2000), for their effect on food intake (G. Colombo et al., Life Sciences, 63 (8), 113-117 (1998); J. Siamand et al., Behavioral Pharmacol., 9, 179-181 (1998)) and for the treatment of Parkinson's disease, epilepsy, migraine and stress (G. Gerdeman, D M. Lovinger, J. Neurophysiol., 85(1), 468-471, 2001; WO 0046209).

Endocannabinoids have been detected in many structures of the brain, including those regions involved with appetite control, movement and memory. Here, they act as neuromodulators via CB1 receptors, frequently causing a pre-synaptic inhibition of another neurotransmitter, which results in reduction in neuronal activity in the structure concerned. Indeed, cannabinoid agonists have been demonstrated to reduce activity in many neurotransmitter systems and to have profound effects on appetite, behavior and coordination and memory. CB1 agonists are known to impair working memory while CB1 antagonists have been shown to reverse working memory deficits.

Various other drugs have been developed for the treatment of psychiatric disorders, particularly in the treatment of schizophrenia. However, quite a few of these drugs exhibit side effects such as weight gain, such as for example olanzapine. A few other psychiatric drugs, for example, haloperidol causes catalepsy. Various drugs that are suitable for treating schizophrenia are also listed in Goodman & Gilman's The Pharmacological Basis of Therapeutics, 9^{th} Ed., McGraw-Hill, 1996, p 404-406.

Thus there is a need for developing a drug either as monotherapy or in combination with other suitable drugs to alleviate certain of the above noted side effects as well as negative symptoms associated in treating various neurological disorders including psychiatric disorders.

All of the references described herein are incorporated herein by reference in their entirety.

### SUMMARY OF THE INVENTION

In one aspect of this invention there is provided a method of treating cognition deficits in a patient suffering from schizophrenia by administering to said patient a therapeutically effective amount of a CB1 receptor antagonist as described hereinbelow.

In another aspect of this invention there is provided a combination of one or more CB1 receptor antagonists and of one or more antipsychotic agent useful in the treatment of psychiatric disorders. The combination of this invention provides synergistic results in that the combination improves positive and negative symptoms of schizophrenia, weight gain and catalepsy.

### DETAILED DESCRIPTION OF THE INVENTION

The terms as used herein have the following meanings:

As used herein, the expression “C₁₋₆alkyl” includes methyl and ethyl groups, and straight-chained or branched propyl, butyl, pentyl and hexyl groups. Particular alkyl groups are methyl, ethyl, n-propyl, isopropyl and tert-butyl. Derived expressions such as “C₁₋₄alkoxy”, “C₁₋₄thioalkyl” “C₁₋₄alkoxyC₁₋₄alkyl”, “hydroxyC₁₋₄alkyl”, “C₁₋₄alkylcarbonyl”, “C₁₋₄alkoxycarbonyl”, “aminoC₁₋₄alkyl”, “C₁₋₄alkylamino”, “C₁₋₄alkylcarbamoylC₁₋₆alkyl”, “C₁₋₄dialkylcarbamoylC₁₋₄alkyl” “mono- or di-C₁₋₄alkylaminoC₁₋₄alkyl”, “aminoC₁₋₄alkylcarbonyl” “diphenylC₁₋₄alkyl”, “phenylC₁₋₄alkyl”, “phenylcarbonylC₁₋₄alkyl” and “phenoxyC₁₋₄alkyl” are to be construed accordingly.

As used herein, the expression “cycloalkyl” includes all of the known cyclic radicals. Representative examples of “cycloalkyl” includes without any limitation cyclopropyl, cyclobutyl, cyclopentyl, cyclohexyl, cycloheptyl, cyclooctyl, and the like. Derived expressions such as “cycloalkoxy”, “cycloalkylalkyl”, “cycloalkylaryl”, “cycloalkylcarbonyl” are to be construed accordingly.

As used herein, the expression “C₂₋₆alkenyl” includes ethenyl and straight-chained or branched propenyl, butenyl, pentenyl and hexenyl groups. Similarly, the expression “C₂₋₆alkynyl” includes ethynyl and propynyl, and straight-chained or branched butynyl, pentynyl and hexynyl groups.

As used herein the expression “C₁₋₄acyl” shall have the same meaning as “C₁₋₆alkanoyl”, which can also be represented structurally as “R—CO—,” where R is a C₁₋₃alkyl as defined herein. Additionally, “C₁₋₃alkylcarbonyl” shall mean same as C₁₋₄acyl. Specifically, “C₁₋₄acyl” shall mean formyl, acetyl or ethanoyl, propanoyl, n-butanoyl, etc. Derived expressions such as “C₁₋₄acyloxy” and “C₁₋₄-acyloxyalkyl” are to be construed accordingly.

As used herein, the expression “C₁₋₆ perfluoroalkyl” means that all of the hydrogen atoms in said alkyl group are replaced with fluorine atoms. Illustrative examples include trifluoromethyl and pentafluoroethyl, and straight-chained or branched heptafluoropropyl, nonafluorobutyl, undecafluoropentyl and tridecafluorohexyl groups. Derived expression, “C₁₋₆ perfluoroalkoxy”, is to be construed accordingly.

As used herein, the expression “C₆₋₁₂aryl” means substituted or unsubstituted phenyl or naphthyl. Specific examples of substituted phenyl or naphthyl include o-, p-, m-tolyl, 1,2-, 1,3-, 1,4-xylyl, 1-methylnaphthyl, 2-methylnaphthyl, etc. “Substituted phenyl” or “substituted naphthyl” also include any of the possible substituents as further defined herein or one known in the art. Derived expression, “C₆₋₁₂arylsulfonyl,” is to be construed accordingly.

As used herein, the expression “C₆₋₁₂arylC₁₋₄alkyl” means that the C₆₋₁₂aryl as defined herein is further attached to C₁₋₄alkyl as defined herein. Representative examples include benzyl, phenylethyl, 2-phenylpropyl, 1-naphthylmethyl, 2-naphthylmethyl and the like.

As used herein, the expression “heteroaryl” includes all of the known heteroatom containing aromatic radicals. Representative 5-membered heteroaryl radicals include furanyl, thienyl or thiophenyl, pyrrolyl, isopyrrolyl, pyrazolyl, imidazolyl, oxazolyl, thiazolyl, isothiazolyl, and the like. Representative 6-membered heteroaryl radicals include pyridinyl, pyridazinyl, pyrimidinyl, pyrazinyl, triazinyl, and the like radicals. Representative examples of bicyclic heteroaryl radicals include benzofuranyl, benzothiophenyl, indolyl, quinolinyl, isoquinolinyl, cinnolyl, benzimidazolyl, indazolyl, pyridofuranyl, pyridothienyl, and the like radicals.

As used herein, the expression “heterocycle” includes all of the known reduced heteroatom containing cyclic radicals. Representative 5-membered heterocycle radicals include tetrahydrofuranyl, tetrahydrothiophenyl, pyrrolidinyl, 2-thiazolinyl, tetrahydrothiazolyl, tetrahydrooxazolyl, and the like. Representative 6-membered heterocycle radicals include piperidinyl, piperazinyl, morpholinyl, thiomorpholinyl, and the like. Various other heterocycle radicals include, without limitation, aziridinyl, azepanyl, diazepanyl, diazabicyclo[2.2.1]hept-2-yl, and triazocanyl, and the like.

“Halogen” or “halo” means chloro, fluoro, bromo, and iodo.

As used herein, “patient” means a warm blooded animal, such as for example rat, mice, dogs, cats, guinea pigs, and primates such as humans.

As used herein, the expression “pharmaceutically acceptable carrier” means a non-toxic solvent, dispersant, excipient, adjuvant, or other material which is mixed with the compound of the present invention in order to permit the formation of a pharmaceutical composition, i.e., a dosage form capable of administration to the patient. One example of such a carrier is pharmaceutically acceptable oil typically used for parenteral administration.

The term “pharmaceutically acceptable salts” as used herein means that the salts of the compounds of the present invention can be used in medicinal preparations. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, hydrobromic acid, hydroiodic acid, sulfuric acid, methanesulfonic acid, ethanesulfonic acid, 2-hydroxyethanesulfonic acid, benzenesulfonic acid, p-toluenesulfonic acid, gluconic acid, isethionic acid, maleic acid, methylenebis(oxynaphthoic) acid, nitric acid, oxalic acid, palmoic acid, phosphoric acid, salicylic acid, succinic acid, tartaric acid, theophyllineacetic acid, fumaric acid, hydroxymaleic acid, malic acid, ascorbic acid, glutaric acid, acetic acid, cinnamic acid, 2-phenoxybenzoic acid, hydroxybenzoic acid, phenylacetic acid, benzoic acid, oxalic acid, citric acid, glycolic acid, lactic acid, pyruvic acid, malonic acid or carbonic acid. The acid metal salts such as sodium monohydrogen orthophosphate and potassium hydrogen sulfate can also be formed. Also, the salts so formed may present either as mono- or di-acid salts and can exist substantially anhydrous or can be hydrated. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts, and salts formed with suitable organic ligands, e.g. quaternary ammonium salts.

The expression “stereoisomers” is a general term used for all isomers of the individual molecules that differ only in the orientation of their atoms in space. Typically it includes mirror image isomers that are usually formed due to at least one asymmetric center, (enantiomers). Where the compounds according to the invention possess two or more asymmetric centers, they may additionally exist as diastereoisomers, also certain individual molecules may exist as geometric isomers (cis/trans). Similarly, certain compounds of this invention may exist in a mixture of two or more structurally distinct forms that are in rapid equilibrium, commonly known as tautomers. Representative examples of tautomers include keto-enol tautomers, phenol-keto tautomers, nitroso-oxime tautomers, imine-enamine tautomers, etc. It is to be understood that all such isomers and mixtures thereof in any proportion are encompassed within the scope of the present invention.

The term “solvate” as used herein means an aggregate that consists of a solute ion or molecule with one or more solvent molecules. Similarly, a “hydrate” means a solute ion or molecule with one or more water molecules.

In a broad sense, the term “substituted” is contemplated to include all permissible substituents of organic compounds. In a few of the specific embodiments as disclosed herein, the term “substituted” means substituted with one or more substituents independently selected from the group consisting of C₁₋₆alkyl, C₂₋₆alkenyl, C₁₋₆ perfluoroalkyl, phenyl, hydroxy, —CO₂H, an ester, an amide, C₁-C₆alkoxy, C₁-C₆thioalkyl, C₁-C₆ perfluoroalkoxy, —NH₂, Cl, Br, I, F, —NH-lower alkyl, and —N(lower alkyl)₂. However, any of the other suitable substituents known to one skilled in the art can also be used in these embodiments.

“Therapeutically effective amount” means an amount of the compound which is effective in treating the named disease, disorder or condition.

The term “treating” refers to:
(i) preventing a disease, disorder or condition from occurring in a patient that may be predisposed to the disease, disorder and/or condition, but has not yet been diagnosed as having it;
(ii) inhibiting the disease, disorder or condition, i.e., arresting its development; and
(iii) relieving the disease, disorder or condition, i.e., causing regression of the disease, disorder and/or condition.

As used herein “psychiatric disorders” shall have the same meaning as “psychotic disorder” as defined in Diagnostic and Statistical Manual of Mental Disorders, 4^{th} Ed., (“DSM-IV”) American Psychiatric Association, 1995, incorporated herein by reference. The essential feature of brief psychotic disorder is a disturbance that involves the sudden onset of at least one of the following positive psychotic symptoms: delusions, hallucinations, disorganized speech (e.g., frequent derailment or incoherence), or grossly disorganized or catatonic behavior (Criterion A). An episode of the disturbance lasts at least one day but less than one month, and the individual eventually has a full return to the premorbid level of functioning (Criterion B). The disturbance is not better accounted for by a mood disorder with psychotic features, by schizoaffective disorder, or by schizophrenia and is not due to the direct physiological effects of a substance (e.g., hallucinogen) or a general medical condition (e.g., subdural hematoma) (Criterion C).

As used herein “catalepsy” shall mean a failure to correct an externally imposed, unusual posture over a prolonged period of time.

A subject-matter of the invention is therefore a method of treating cognition deficits in a patient suffering from schizophrenia by administering to said patient a therapeutically effective amount of a CB1 antagonist, azetidine derivatives of formula (I) as described hereinbelow.

Use may in particular be made, among CB1 antagonists, of the azetidine derivatives of formula (I). The compounds of formula (I) are disclosed in patent applications: FR 0002775, FR 0002777, FR 0002776 as well as in the corresponding United States patents: S U.S. Pat. No. 6,479,479; U.S. Pat. No. 6,355,631; and U.S. Pat. No. 6,566,356, all of which are incorporated herein by reference in their entirety. The azetidine derivatives used in the present invention are the compounds having the following formula (I):

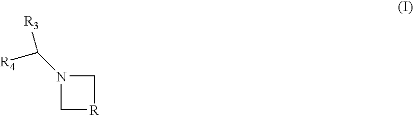

in which
either A:
R represents a CR₁R₂, C═C(R₅)SO₂R₆ or C═C(R₇)SO₂alk radical,
either R₁ represents a hydrogen atom and R₂ represents a —C(R₈)(R₉)(R₁₀), —C(R₈)(R₁₁)(R₁₂), —CO—NR₁₃R₁₄, —CH₂—CO—NR₁₃R₁₄, —CH₂—CO—R₆, —CO—R₆, —CO-cycloalkyl, —SO—R₆, —SO₂—R₆, —C(OH)(R₁₂)(R₆), —C(OH)(R₆)(alkyl), —C(═NOalk)R₆, —C(═NO—CH₂—CH═CH₂)R₆, —CH₂—CH(R₆)NR₃₁R₃₂, —CH₂—C(═NOalk)R₆, —CH(R₆)NR₃₁R₃₂, —CH(R₆)NHSO₂alk, —CH(R₆)NHCONHalk or —CH(R₆)NHCOalk radical,
or R₁ represents an alkyl, NH—R₁₅, cyano, —S-alk-NR₁₆R₁₇, —CH₂—NR₁₈R₁₉ or —NR₂₀R₂₁ radical and R₂ represents a —C(R₈)(R₁₁)(R₁₂) radical,
R₃ and R₄, which are identical or different, represent either an alkyl or cycloalkyl radical, or an aromatic radical chosen from phenyl, naphthyl or indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₂₂R₂₃, —CO—NH—NR₂₄R₂₅, alkylsulfanyl, alkylsulfinyl, alkylsulfonyl, alkylsulfanylalkyl, alkylsulfinylalkyl, alkylsulfonylalkyl, hydroxyalkyl or -alk-NR₂₄R₂₅; or a heteroaromatic radical chosen from the benzofuryl, benzothiazolyl, benzothienyl, benzoxazolyl, chromanyl, 2,3-dihydroxybenzofuryl, 2,3-dihydrobenzothienyl, furyl, imidazolyl, isochromanyl, isoquinolyl, pyrrolyl, pyridyl, pyrimidinyl, quinolyl, 1,2,3,4-tetrahydro-isoquinolyl, thiazolyl and thienyl rings, it being possible for these heteroaromatic radicals to be unsubstituted or substituted by one or more halogen, alkyl, alkoxy, hydroxyl, trifluoromethyl, trifluoromethoxy, cyano, —COOH, —COOalk, —CO—NH—NR₂₄R₂₅, —CONR₂₂R₂₃, -alk-NR₂₄R₂₅, alkylsulfanyl, alkylsulfinyl, alkylsulfonyl, alkylsulfanylalkyl, alkylsulfinylalkyl, alkylsulfonylalkyl or hydroxyalkyl,
R₅ represents a hydrogen atom or an alkyl radical,
R₆ represents an Ar₁ or Het₁ radical,
R₇ represents a cycloalkyl, heterocycloalkyl or heterocyclenyl radical optionally substituted by a —CSO-phenyl radical,
R₅ represents a hydrogen atom or an alkyl radical,
R₉ represents a —CO—NR₂₆R₂₇, —COOH, —COOalk, —CH₂OH, —NH—CO—NH-alk, —CH₂—NHR₂₈ or —NHCOOalk radical,
R₁₀ represents an Ar₁ or Het₁ radical,
R₁₁ represents an —SO₂-alk, —SO₂—Ar₁ or —SO₂-Het₁ radical,
R₁₂ represents a hydrogen atom or an Ar₁ or Het₁ radical,
R₁₃ represents a hydrogen atom or an alkyl radical,
R₁₄ represents an Ar₁, Het₁, -alk-Ar₁ or -alk-Het₁ radical,
R₁₅ represents an alkyl, cycloalkyl or -alk-NR₂₉R₃₀ radical,
R₁₆ and R₁₇, which are identical or different, represent a hydrogen atom or an alkyl radical or else R₁₆ and R₁₇ form, together with the nitrogen atom to which they are attached, a saturated or unsaturated and mono- or bicyclic heterocycle having 3 to 10 ring members and optionally comprising one or more other heteroatoms chosen from oxygen, sulfur and nitrogen and optionally substituted by one or more alkyl radicals,
R₁₈ represents a hydrogen atom or an alkyl radical,
R₁₉ represents a hydrogen atom or an alkyl, cycloalkyl, cycloalkylalkyl, cycloalkylcarbonyl, —SO₂alk, —CO—NHalk or —COOalk radical,
or else, R₁₈ and R₁₉ form, with the nitrogen atom to which they are attached, a saturated or unsaturated and mono- or bicyclic heterocycle having 3 to 10 ring members and optionally comprising one or more heteroatoms chosen from oxygen, sulfur and nitrogen and optionally substituted by one or more alkyl radicals,
—NR₂₀R₂₁ represents a saturated or unsaturated monocyclic heterocycle having 3 to 8 ring members and optionally comprising another heteroatom chosen from oxygen, nitrogen and sulfur,
R₂₂ and R₂₃, which are identical or different, represent a hydrogen atom or an alkyl radical or else R₂₂ and R₂₃ form, together with the nitrogen atom to which they are attached, a saturated mono- or bicyclic heterocycle having 3 to 10 ring members and optionally comprising another heteroatom chosen from oxygen, sulfur and nitrogen and optionally being substituted by one more alkyl radicals,
R₂₄ and R₂₅, which are identical or different, represent a hydrogen atom or an alkyl, —COOalk, cycloalkyl, alkylcycloalkyl, -alk-O-alk or hydroxyalkyl radical or else R₂₄ and R₂₅ form, together with the nitrogen atom to which they are attached, a saturated or unsaturated and mono- or bicyclic heterocycle having 3 to 10 ring members and optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂ radicals,
R₂₆ and R₂₇, which are identical or different, represent a hydrogen atom or an alkyl, hydroxyalkyl, cycloalkyl, cycloalkylalkyl, -alk-COOalk, -alk-Ar₁, alk-Het₁, Het₁ or -alk-N(alk)₂ radical, R₂₆ and R₂₇ can also form, with the nitrogen atom to which they are attached, a saturated or unsaturated and mono- or bicyclic heterocycle having 3 to 10 ring members and optionally comprising one or more heteroatoms chosen from oxygen, sulfur and nitrogen and optionally substituted by one or more alkyl, alkoxy or halogen radicals,
R₂₈ represents a —CH₂-alk, benzyl, —SO₂alk, —CONHalk, —COalk, cycloalkylalkylcarbonyl, cycloalkylcarbonyl or —CO—(CH₂)ₙOH radical,
n is equal to 1, 2, or 3,
R₂₉ and R₃₀, which are identical or different, represent a hydrogen atom or an alkyl radical or else R₂₉ and R₃₀ form, together with the nitrogen atom to which they are attached, a saturated mono- or bicyclic heterocycle having 3 to 10 ring members and optionally comprising another heteroatom chosen from oxygen, sulfur and nitrogen and optionally being substituted by one or more alkyl radicals,
R₃₁ and R₃₂, which are identical or different, represent a hydrogen atom or an alkyl, Ar₁ or -alk-Ar₁ radical or else R₃₁ and R₃₂ form, together with the nitrogen atom to which they are attached, a heterocycle chosen from aziridinyl, azetidinyl, pyrrolidinyl and piperidinyl,
Ar₁ represents a phenyl or naphthyl radical optionally substituted by one or more substituents chosen from halogen, alkyl, alkoxy, —CO-alk, cyano, —COOH, —COOalk, —CONR₂₂R₂₃, —CO—NH—NR₂₄R₂₅, alkylsulfanyl, alkylsulfinyl, alkylsulfonyl, alkylsulfanylalkyl, alkylsulfinylalkyl, alkylsulfonylalkyl, hydroxyalkyl, -alk-NR₂₄R₂₅, —NR₂₄R₂₅, alkylthioalkyl, formyl, hydroxyl, CF₃, OCF₃, Het₁, O-alk-NH-cycloalkyl or SO₂NH₂,
Het₁ represents a saturated or unsaturated and mono- or bicyclic heterocycle having 3 to 10 ring members and comprising one or more heteroatoms chosen from oxygen, sulfur and nitrogen and optionally substituted by one or more halogen, allyl, alkoxy, alkoxycarbonyl, —CONR₂₂R₂₃, hydroxyl, hydroxyalkyl, oxo or SO₂NH₂, or B:
R represents a CHR₃₃ radical,
R₃₃ represents an —NHCOR₃₄ or —N(R₃₅)—Y—R₃₆ radical,
Y is CO or SO₂,
R₃ and R₄, which are identical or different, represent either an aromatic radical chosen from phenyl, naphthyl and indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₃₇R₃₈, —CO—NH—NR₃₉R₄₀, alkylsulfanyl, alkylsulfinyl, alkylsulfonyl, alkylsulfanylalkyl, alkylsulfinylalkyl, alkylsulfonylalkyl, hydroxyalkyl or -alk-NR₃₇R₃₈; or a heteroaromatic radical chosen from the benzofuryl, benzothiazolyl, benzothienyl, benzoxazolyl, chromanyl, 2,3-dihydrobenzofuryl, 2,3-dihydrobenzothienyl, pyrimidinyl, furyl, imidazolyl, isochromanyl, isoquinolyl, pyrrolyl, pyridyl, quinolyl, 1,2,3,4-tetrahydroisoquinolyl, thiazolyl and thienyl rings, it being possible for these heteroaromatic radicals to be unsubstituted or substituted by a halogen, alkyl, alkoxy, hydroxyl, trifluoromethyl, trifluoromethoxy, cyano, —COOH, —COOalk, —CO—NH—NR₃₉R₄₀, —CONR₃₇R₃₈, -alk-NR₃₉R₄₀, alkylsulfanyl, alkylsulfinyl, alkylsulfonyl, alkylsulfanylalkyl, alkylsulfinylalkyl, alkylsulfonylalkyl or hydroxyalkyl,
R₃₄ represents an -alk-SO₂—R₄₁ radical, an -alk-SO₂—CH═CH—R₄₁ radical, a Het₂ radical substituted by —SO₂—R₄₁ or a phenyl radical substituted by —SO₂—R₄₁ or -alk-SO₂—R₄₁,
R₃₅ represents a hydrogen atom or an alkyl radical,
R₃₆ represents a phenylalkyl, Het₂ or Ar₂ radical,
R₃₇ and R₃₈, which are identical or different, represent a hydrogen atom or an alkyl radical or else R₃₇ and R₃₈ form, together with the nitrogen atom to which they are attached, a saturated mono- or bicyclic heterocycle having 3 to 10 ring members and optionally comprising another heteroatom chosen from oxygen, sulfur and nitrogen and optionally being substituted by one or more alkyl,
R₃₉ and R₄₀, which are identical or different, represent a hydrogen atom or an alkyl, —COOalk, cycloalkyl, alkylcycloalkyl, -alk-O-alk or hydroxyalkyl radical or else R₃₉ and R₄₀ form, together with the nitrogen atom to which they are attached, a saturated or unsaturated and mono- or bicyclic heterocycle having 3 to 10 ring members and optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂,
R₄₁ represents an alkyl, Ar₂ or Het₂ radical,
Ar₂ represents a phenyl, naphthyl or indenyl radical, these radicals optionally being substituted by one or more halogen, alkyl, alkoxy, cyano, —CO-alk, —COOH, —COOalk, —CONR₄₂R₄₃, —CO—NH—NR₄₄R₄₅, alkylsulfanyl, alkylsulfinyl, alkylsulfonyl, -alk-NR₄₄R₄₅, —NR₄₄R₄₅, alkylthioalkyl, formyl, hydroxyl, hydroxyalkyl, Het₂, —O-alk-NH-cycloalkyl, OCF₃, CF₃, —NH—CO-alk, —SO₂NH₂, —HN—COCH₃, —NH—COOalk or Het₂ or else on two adjacent carbon atoms by a dioxymethylene,
Het₂ represents a saturated or unsaturated and mono- or bicyclic heterocycle having 3 to 10 ring members and comprising one or more heteroatoms chosen from oxygen, sulfur and nitrogen optionally substituted by one or more alkyl, alkoxy, vinyl, halogen, alkoxycarbonyl, oxo, hydroxyl, OCF₃ or CF₃, the nitrogenous heterocycles optionally being in their N-oxidized form,
R₄₂ and R₄₃, which are identical or different, represent a hydrogen atom or an alkyl radical or else R₄₂ and R₄₃ form, together with the nitrogen atom to which they are attached, a saturated mono- or bicyclic heterocycle having 3 to 10 ring members and optionally comprising another heteroatom chosen from oxygen, sulfur and nitrogen and optionally being substituted by one or more alkyl radicals,
R₄₄ and R₄₅, which are identical or different, represent a hydrogen atom or an alkyl, —COOalk, cycloalkyl, alkylcycloalkyl, -alk-O-alk or hydroxyalkyl radical or else R₄₄ and R₄₅ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂ radicals, or C:
R represents a CHR₄₆ radical,
R₄₆ represents an —N(R₄₇)R₄₈, —N(R₄₇)—CO—R₄₈ or —N(R₄₇)—SO₂R₄₉ radical,
R₃ and R₄, which are identical or different, represent either an aromatic radical chosen from phenyl, naphthyl and indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₅₀R₅₁, —CO—NH—NR₅₂R₅₃, alkylsulfanyl, alkylsulfinyl, alkylsulfonyl, alkylsulfanylalkyl, alkylsulfinylalkyl, alkylsulfonylalkyl, hydroxyalkyl or -alk-NR₇R₈ radicals; or a heteroaromatic radical chosen from the benzofuryl, benzothiazolyl, benzothienyl, benzoxazolyl, chromanyl, 2,3-dihydrobenzofuryl, 2,3-dihydrobenzothienyl, furyl, imidazolyl, isochromanyl, isoquinolyl, pyrrolyl, pyridyl, pyrimidyl, quinolyl, 1,2,3,4-tetrahydroisoquinolyl, thiazolyl and thienyl rings, it being possible for these heteroaromatic radicals to be unsubstituted or substituted by a halogen, alkyl, alkoxy, hydroxyl, trifluoromethyl, trifluoromethoxy, cyano, —COOH, —COOalk, —CO—NH—NR₅₂R₅₃, —CONR₅₀R₅₁, -alk-NR₅₂R₅₃, alkylsulfanyl, alkylsulfinyl, alkylsulfonyl, alkylsulfanylalkyl, alkylsulfinylalkyl, alkylsulfonylalkyl or hydroxyalkyl radical,
R₄₇ represents a —C(R₅₄)(R₅₅)-Het₃, -Het₃, —C(R₅₄)(R₅₅)—Ar₃, Ar₃, cycloalkyl or norbornyl radical,
R₄₈ represents a hydrogen atom or a hydroxyalkyl radical, -alk-COOalk radical, -alk-CONR₅₀R₅₁ radical, -alk-NR₅₀R₅₁ radical, alkoxy radical, Ar₃ radical, Het₃ radical, —CH₂Ar₃ radical, —CH₂Het₃ radical or alkyl radical optionally substituted with one or more halogen,
R₄₉ represents a hydroxyalkyl radical, -alk-COOalk radical, -alk-CONR₅₀R₅₁ radical, -alk-NR₅₀R₅₁ radical, alkoxy radical, Ar₃ radical, Het₃ radical, —CH₂Ar₃ radical, —CH₂Het₃ radical or alkyl radical optionally substituted with one or more halogen,
R₅₀ and R₅₁, which are identical or different, represent a hydrogen atom or an alkyl radical or else R₅₀ and R₅₁ form, together with the nitrogen atom to which they are attached, a saturated mono- or bicyclic heterocycle having 3 to 10 ring members and optionally comprising another heteroatom chosen from oxygen, sulfur and nitrogen and optionally being substituted by one or more alkyl,
R₅₂ and R₅₃, which are identical or different, represent a hydrogen atom or an alkyl, —COOalk, cycloalkyl, alkylcycloalkyl, -alk-O-alk or hydroxyalkyl radical or else R₅₂ and R₅₃ form, together with the nitrogen atom to which they are attached, a saturated or unsaturated and mono- or bicyclic heterocycle having 3 to 10 ring members and optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂,
R₅₄ represents a hydrogen atom or a hydroxyalkyl radical, -alk-COOalk radical, -alk-CONR₅₀R₅₁ radical, -alk-NR₅₀R₅₁ radical, alkoxyalkyl radical, Ar₃ radical, Het₃ radical, —CH₂Ar₃ radical, —CH₂Het₃ radical or alkyl radical optionally substituted with one or more halogen,
R₅₅ represents a hydrogen atom or a hydroxyalkyl radical, -alk-COOalk radical, -alk-CONR₅₀R₅₁ radical, -alk-NR₅₀R₅₁ radical, alkoxyalkyl radical or alkyl radical optionally substituted with one or more halogen,
or else R₅₄ and R₅₅ form, together with the carbon atom to which they are attached, a saturated mono- or bicyclic ring having 3 to 10 ring members and optionally comprising another heteroatom chosen from oxygen, sulfur and nitrogen and optionally being substituted by one or more alkyl,
Ar₃ represents a phenyl, naphthyl or indenyl radical, these various radicals optionally being substituted by one or more halogen, alkyl, alkoxy, —CO-alk, cyano, —COOH, —COOalk, —CONR₅₆R₅₇, —CO—NH—NR₅₈R₅₉, alkylsulfanyl, alkylsulfinyl, alkylsulfonyl, -alk-NR₅₈R₅₉, —NR₅₈R₅₉, alkylthioalkyl, formyl, CF₃, OCF₃, Het₃, —O-alk-NH-cycloalkyl, SO₂NH₂, hydroxyl, hydroxyalkyl, —NHCOalk or —NHCOOalk or on 2 adjacent carbon atoms by dioxymethylene,
Het₃ represents a saturated or unsaturated and mono- or bicyclic heterocycle having 3 to 10 ring members and comprising one or more heteroatoms chosen from oxygen, sulfur and nitrogen and optionally substituted by one or more alkyl, alkoxy, halogen, alkoxycarbonyl, oxo or hydroxyl, the nitrogenous heterocycles optionally being in their N-oxidized form,
R₅₆ and R₅₇, which are identical or different, represent a hydrogen atom or an alkyl radical or else R₅₆ and R₅₇ form, together with the nitrogen atom to which they are attached, a saturated mono- or bicyclic heterocycle having 3 to 10 ring members and optionally comprising another heteroatom chosen from oxygen, sulfur and nitrogen and optionally being substituted by one or more alkyl,
R₅₈ and R₅₉, which are identical or different, represent a hydrogen atom or an alkyl radical or else R₅₈ and R₅₉ form, together with the nitrogen atom to which they are attached, a saturated mono- or bicyclic heterocycle having 3 to 10 ring members and optionally comprising another heteroatom chosen from oxygen, sulfur and nitrogen and optionally being substituted by one or more alkyl,
alk represents an alkyl or alkylene radical,
the alkyl and alkylene radicals as well as the alkoxy radicals may feature straight or branched chains and comprise 1 to 6 carbon atoms, the cycloalkyl radicals comprise 3 to 10 carbon atoms and the heterocycloalkyl and heterocyclenyl radicals comprise 3 to 10 carbon atoms,
the optical isomers of these compounds and their pharmaceutically acceptable salts with an inorganic or organic acid.

Representative examples of specific CB1 antagonists within the scope of this invention without any limitation include the following:
(RS)-1-[bis(4-chlorophenyl)methyl)]-3-[(3,5-difluoro-phenyl)(methylsulfonyl)methyl]azetidine,
(R)-1-[bis(4-chlorophenyl)methyl)]-3-[(3,5-difluoro-phenyl)(methylsulfonyl)methyl]azetidine,
(S)-1-[bis(4-chlorophenyl)methyl)]-3-[(3,5-difluoro-phenyl)(methylsulfonyl)methyl]azetidine,
(RS)-1-[bis(4-chlorophenyl)methyl)]-3-[(pyrid-3-yl)-(methylsulfonyl)methyl]azetidine,
(R)-1-[bis(4-chlorophenyl)methyl)]-3-[(pyrid-3-yl)-(methylsulfonyl)methyl]azetidine,
(S)-1-[bis(4-chlorophenyl)methyl)]-3-[(pyrid-3-yl)-(methylsulfonyl)methyl]azetidine,
(RS)-1-[bis(3-fluorophenyl)methyl]-3-[(3,5-difluorophenyl)(methylsulfonyl)methyl]azetidine,
(R)-1-[bis(3-fluorophenyl)methyl]-3-[(3,5-difluorophenyl)(methylsulfonyl)methyl]azetidine,
(S)-1-[bis(3-fluorophenyl)methyl]-3-[(3,5-difluorophenyl)(methylsulfonyl)methyl]azetidine,
1-[bis(4-chlorophenyl)methyl]-3-(RS)-{[3-(azetidin-1-yl)phenyl](methylsulfonyl)methyl}azetidine,
1-[bis(4-chlorophenyl)methyl]-3-(R)-{[3-(azetidin-1-yl)phenyl](methylsulfonyl)methyl}azetidine,
1-[bis(4-chlorophenyl)methyl]-3-(S)-{[3-(azetidin-1-yl)phenyl](methylsulfonyl)methyl}azetidine,
(RS)-1-[3-({1-[bis(4-chlorophenyl)methyl]azetidin-3-yl}(methylsulfonyl)methyl)phenyl]pyrrolidine,
(R)-1-[3-({1-[bis(4-chlorophenyl)methyl]azetidin-3-yl}(methylsulfonyl)methyl)phenyl]pyrrolidine,
(S)-1-[3-({1-[bis(4-chlorophenyl)methyl]azetidin-3-yl}(methylsulfonyl)methyl)phenyl]pyrrolidine,
(RS)—N-[3-({1-[bis(4-chlorophenyl)methyl]azetidin-3-yl}(methylsulfonyl)methyl)phenyl]-N-methylamine,
(R)—N-[3-({1-[bis(4-chlorophenyl)methyl]azetidin-3-yl}(methylsulfonyl)methyl)phenyl]-N-methylamine,
(S)—N-[3-({1-[bis(4-chlorophenyl)methyl]azetidin-3-yl}(methylsulfonyl)methyl)phenyl]-N-methylamine,
(RS)-1-[bis(4-chlorophenyl)methyl]-3-[(3,5-bis(trifluoro-methyl)phenyl)(methylsulfonyl)methyl]azetidine,
(R)-1-[bis(4-chlorophenyl)methyl]-3-[(3,5-bis(trifluoro-methyl)phenyl)(methylsulfonyl)methyl]azetidine,
(S)-1-[bis(4-chlorophenyl)methyl]-3-[(3,5-bis(trifluoro-methyl)phenyl)(methylsulfonyl)methyl]azetidine,
1-[bis(4-chlorophenyl)methyl]-3-(phenylsulfonyl-methyl)azetidine,
(RS)-1-[bis(4-chlorophenyl)methyl]-3-[(3,5-difluoro-phenyl)(methylsulfonyl)methyl]-3-methylazetidine,
(R)-1-[bis(4-chlorophenyl)methyl]-3-[(3,5-difluorophenyl)methylsulfonylmethyl]-3-methylazetidine,
(S)-1-[bis(4-chlorophenyl)methyl]-3-[(3,5-difluoro-phenyl)(methylsulfonyl)methyl]-3-methylazetidine,
(RS)-2-{1-[bis(4-chlorophenyl)methyl]azetidin-3-yl}-2-(3,5-difluorophenyl)-N-cyclohexylacetamide,
(R)-2-{1-[bis(4-chlorophenyl)methyl]azetidin-3-yl}-2-(3,5-difluorophenyl)-N-cyclohexylacetamide,
(S)-2-{1-[bis(4-chlorophenyl)methyl]azetidin-3-yl}-2-(3,5-difluorophenyl)-N-cyclohexylacetamide,
(RS)-2-{1-[bis(4-chlorophenyl)methyl]azetidin-3-yl}-2-(3,5-difluorophenyl)-N-isobutylacetamide,
(R)-2-{1-[bis(4-chlorophenyl)methyl]azetidin-3-yl}-2-(3,5-difluorophenyl)-N-isobutylacetamide,
(S)-2-{1-[bis(4-chlorophenyl)methyl]azetidin-3-yl}-2-(3,5-difluorophenyl)-N-isobutylacetamide,
(RS)-2-{1-[bis(4-chlorophenyl)methyl]azetidin-3-yl}-2-(3,5-difluorophenyl)-N-cyclopropylmethylacetamide,
(R)-2-{1-[bis(4-chlorophenyl)methyl]azetidin-3-yl}-2-(3,5-difluorophenyl)-N-isopropylacetamide,
(S)-2-{1-[bis(4-chlorophenyl)methyl]azetidin-3-yl}-2-(3,5-difluorophenyl)-N-cyclopropylmethylacetamide,
(RS)-2-{1-[bis(4-chlorophenyl)methyl]azetidin-3-yl}-2-(3,5-difluorophenyl)-N-isopropylacetamide,
(R)-2-{1-[bis(4-chlorophenyl)methyl]azetidin-3-yl}-2-(3,5-difluorophenyl)-N-isopropylacetamide,
(S)-2-{1-[bis(4-chlorophenyl)methyl]azetidin-3-yl}-2-(3,5-difluorophenyl)-N-isopropylacetamide,
(RS)-1-[bis(4-chlorophenyl)methyl]-3-[1-(3,5-difluorophenyl)-1-(methylsulfonyl)ethyl]azetidine,
(R)-1-[bis(4-chlorophenyl)methyl]-3-[1-(3,5-difluorophenyl)-1-(methylsulfonyl)ethyl]azetidine,
(S)-1-[bis(4-chlorophenyl)methyl]-3-[1-(3,5-difluorophenyl)-1-(methylsulfonyl)ethyl]azetidine,
(RS)-1-[bis(4-fluorophenyl)methyl]-3-[(3,5-difluoro-phenyl)(methylsulfonyl)methyl]azetidine,
(R)-1-[bis(4-fluorophenyl)methyl]-3-[(3,5-difluoro-phenyl)(methylsulfonyl)methyl]azetidine,
(S)-1-[bis(4-fluorophenyl)methyl]-3-[(3,5-difluoro-phenyl)(methylsulfonyl)methyl]azetidine,
(RS)-{1-[(3-pyridyl)(4-chlorophenyl)methyl]-3-[(3,5-difluoro-phenyl)(methylsulfonyl)methyl]azetidine,
(SS)-{1-[(3-pyridyl)(4-chlorophenyl)methyl]-3-[(3,5-difluoro-phenyl)(methylsulfonyl)methyl]azetidine,
(RR)-{1-[(3-pyridyl)(4-chlorophenyl)methyl]-3-[(3,5-difluoro-phenyl)(methylsulfonyl)methyl]azetidine,
(SR)-{1-[(3-pyridyl)(4-chlorophenyl)methyl]-3-[(3,5-difluoro-phenyl)(methylsulfonyl)methyl]azetidine,
(RS)-{1-[(4-pyridyl)(4-chlorophenyl)methyl]-3-[(3,5-difluoro-phenyl)(methylsulfonyl)methyl]azetidine,
(SS)-{1-[(4-pyridyl)(4-chlorophenyl)methyl]-3-[(3,5-difluoro-phenyl)(methylsulfonyl)methyl]azetidine,
(RR)-{1-[(4-pyridyl)(4-chlorophenyl)methyl]-3-[(3,5-difluoro-phenyl)(methylsulfonyl)methyl]azetidine,
(SR)-{1-[(4-pyridyl)(4-chlorophenyl)methyl]-3-[(3,5-difluoro-phenyl)(methylsulfonyl)methyl]azetidine,
(RS)-5-((4-chlorophenyl) {3-[(3,5-difluorophenyl)(methylsulfonyl)methyl]azetidin-1-yl}methyl)pyrimidine,
(SR)-5-((4-chlorophenyl) {3-[(3,5-difluorophenyl)(methylsulfonyl)methyl]azetidin-1-yl}methyl)pyrimidine,
(RR)-5-((4-chlorophenyl) {3-[(3,5-difluorophenyl)(methylsulfonyl)methyl]azetidin-1-yl}methyl)pyrimidine,
(SS)-5-((4-chlorophenyl) {3-[(3,5-difluorophenyl)(methylsulfonyl)methyl]azetidin-1-yl}methyl)pyrimidine,
(SS)-{1-[(2-chloropyrid-5-yl)(4-chlorophenyl)methyl]-3-[(3,5-difluorophenyl)(methylsulfonyl)methyl]azetidine,
(RR)-{1-[(2-chloropyrid-5-yl)(4-chlorophenyl)methyl]-3-[(3,5-difluorophenyl)(methylsulfonyl)methyl]azetidine,
(RS)-{1-[(2-chloropyrid-5-yl)(4-chlorophenyl)methyl]-3-[(3,5-difluorophenyl)(methylsulfonyl)methyl]azetidine,
(SR)-{1-[(2-chloropyrid-5-yl)(4-chlorophenyl)methyl]-3-[(3,5-difluorophenyl)(methylsulfonyl)methyl]azetidine,
N-{1-[bis(4-chlorophenyl)methyl]azetidin-3-yl}thien-2-ylsulfonamide,
N-{1-[bis(4-chlorophenyl)methyl]azetidin-3-yl}-4-methoxyphenylsulfonamide,
N-[4-(N-{1-[bis(4-chlorophenyl)methyl]azetidin-3-yl}sulfamoyl)phenyl]acetamide,
N-{1-[bis(4-chlorophenyl)methyl]azetidin-3-yl}-4-methylphenylsulfonamide,
N-{1-[bis(4-chlorophenyl)methyl]azetidin-3-yl}-3,4-dimethoxyphenylsulfonamide,
N-{1-[bis(4-chlorophenyl)methyl]azetidin-3-yl}-3-fluorophenylsulfonamide,
N-{1-[bis(4-chlorophenyl)methyl]azetidin-3-yl}-3,4-dichlorophenylsulfonamide,
N-{1-[bis(4-chlorophenyl)methyl]azetidin-3-yl}-3-cyanophenylsulfonamide,
N-{1-[bis(4-chlorophenyl)methyl]azetidin-3-yl}-2,5-dimethoxyphenylsulfonamide,
N-{1-[bis(4-chlorophenyl)methyl]azetidin-3-yl}-3-trifluoromethylphenylsulfonamide,
N-{1-[bis(4-chlorophenyl)methyl]azetidin-3-yl}naphth-2-ylsulfonamide,
N-{1-[bis(4-chlorophenyl)methyl]azetidin-3-yl}naphth-1-ylsulfonamide,
N-{1-[bis(4-chlorophenyl)methyl]azetidin-3-yl}-3,4-difluorophenylsulfonamide,
N-{1-[bis(4-chlorophenyl)methyl]azetidin-3-yl}-1-methyl-1H-imidazol-4-ylsulfonamide,
N-[4-(N-{1-[bis(4-chlorophenyl)methyl]azetidin-3-yl}sulfamoyl)-2-chlorophenyl]acetamide,
N-{1-[bis(4-chlorophenyl)methyl]azetidin-3-yl}pyrid-3-ylsulfonamide,
N-{1-[bis(4-chlorophenyl)methyl]azetidin-3-yl}-4-fluorophenylsulfonamide,
N-{1-[bis(4-chlorophenyl)methyl]azetidin-3-yl}quinol-8-ylsulfonamide,
N-{1-[bis(4-chlorophenyl)methyl]azetidin-3-yl}phenylsulfonamide,
N-{1-[bis(4-chlorophenyl)methyl]azetidin-3-yl}(phenylmethyl)sulfonamide,
N-{1-[bis(4-chlorophenyl)methyl]azetidin-3-yl}-3,5-difluorophenylsulfonamide,
N-{1-[bis(4-chlorophenyl)methyl]azetidin-3-yl}pyrid-2-ylsulfonamide,
N-{1-[bis(4-chlorophenyl)methyl]azetidin-3-yl}-(3-fluoro-5-pyrrolidin-1-ylphenyl)sulfonamide,
N-{1-[bis(4-chlorophenyl)methyl]azetidin-3-yl}-N-methyl-4-fluorophenylsulfonamide,
N-{1-[bis(4-chlorophenyl)methyl]azetidin-3-yl}-N-methylquinol-8-ylsulfonamide,
N-{1-[bis(4-chlorophenyl)methyl]azetidin-3-yl}-N-methylphenylsulfonamide,
N-{1-[bis(4-chlorophenyl)methyl]azetidin-3-yl}-N-methyl(phenylmethyl)sulfonamide,
N-{1-[bis(4-chlorophenyl)methyl]azetidin-3-yl}-3-sulfamoylphenylsulfonamide,
2-benzenesulfonyl-N-{1-[bis(4-chlorophenyl)methyl]azetidin-3-yl}acetamide,
N-{1-[bis(4-chlorophenyl)methyl]azetidin-3-yl}-2-(toluene-4-sulfonyl)acetamide,
(3-chloro-4-(methylsulfonyl)thiophene-2-carboxy) {1-[bis(4-chlorophenyl)methyl]azetidin-3-yl}amide,
N-{1-[bis(4-chlorophenyl)methyl]azetidin-3-yl}-3-(2-phenylethylenesulfonyl)propionamide,
N-{1-[bis(4-chlorophenyl)methyl]azetidin-3-yl}-4-(methylsulfonyl)benzamide,
(5-(methylsulfonyl)thiophene-2-carboxy)-{1-[bis(4-chlorophenyl)methyl]azetidin-3-yl}amide,
(5-(methylsulfonyl)-3-methyl-4-vinylthiophene-2-carboxy) {1-[bis(4-chlorophenyl)methyl]azetidin-3-yl}amide,
(RS)—N-{1-[{4-chlorophenyl)(pyridin-3-yl)methyl]azetidin-3-yl}-3,5-difluorobenzenesulfonamide,
(RS)—N-{1-[{4-chlorophenyl)(pyrimidin-5-yl)methyl]azetidin-3-yl}-3,5-difluorobenzenesulfonamide,
N-{1-[bis{4-chlorophenyl)methyl]azetidin-3-yl}-N-(6-chloropyrid-2-yl)methylsulfonamide,
N-{1-[bis{4-chlorophenyl)methyl]azetidin-3-yl}-N-(6-ethylpyrid-2-yl)methylsulfonamide,
N-{1-[bis{4-chlorophenyl)methyl]azetidin-3-yl}-N-(quinol-6-yl)methylsulfonamide,
N-{1-[bis{4-chlorophenyl)methyl]azetidin-3-yl}-N-(quinol-5-yl)methylsulfonamide,
N-{1-[bis{4-chlorophenyl)methyl]azetidin-3-yl}-N-(isoquinol-5-yl)methylsulfonamide,
N-{1-[bis{4-chlorophenyl)methyl]azetidin-3-yl}-N-(pyrid-3-yl)methylsulfonamide,
N-{1-[bis{4-chlorophenyl)methyl]azetidin-3-yl}-N-(1-oxidopyrid-3-yl)methylsulfonamide,
N-((1R,2S,4S)bicyclo[2.2.1]hept-2-yl)-N-{1-[bis(4-chlorophenyl)methyl]azetidin-3-yl}methylsulfonamide,
N-((1R,2R,4S)bicyclo[2.2.1]hept-2-yl)-N-{1-[bis(4-chlorophenyl)methyl]azetidin-3-yl}methylsulfonamide,
N-{1-[bis(4-chlorophenyl)methyl]azetidin-3-yl}-N-(3,5-difluorophenyl)methylsulfonamide,
N-{1-[bis(4-chlorophenyl)methyl]azetidin-3-yl}-N-(thiazol-2-yl)methylsulfonamide,
N-{1-[bis(4-chlorophenyl)methyl]azetidin-3-yl}-N-(3-methoxyphenyl)methylsulfonamide,
N-{1-[bis(4-chlorophenyl)methyl]azetidin-3-yl}-N-(3-(hydroxyphenyl)methylsulfonamide,
N-{1-[bis(4-chlorophenyl)methyl]azetidin-3-yl}-N-(3-(hydroxymethyl)phenyl)methylsulfonamide,
ethyl N-{1-[bis(4-chlorophenyl)methyl]azetidin-3-yl}-N-(methylsulfonyl)-3-aminobenzoate
N-{1-[bis(4-chlorophenyl)methyl]azetidin-3-yl}-N-(1-isobutylpiperid-4-yl)methylsulfonamide,
N-benzyl-N-{1-{bis(4-chlorophenyl)methyl]azetidin-3-yl}amine
N-{1-[bis(4-chlorophenyl)methyl]azetidin-3-yl}-N-(3,5-difluorobenzyl)amine,
N-{1-[bis(4-chlorophenyl)methyl]azetidin-3-yl}-N-(3,5-difluorobenzyl)methylsulfonamide,
N-{1-[bis(4-chlorophenyl)methyl]azetidin-3-yl}-N-(pyrid-3-ylmethyl)methylsulfonamide,
N-{1-[bis(4-fluorophenyl)methyl]azetidin-3-yl}-N-(3,5-difluorophenyl)methylsulfonamide,
(RS)—N-{1-[(4-chlorophenyl)(pyrid-3-yl)methyl]azetidin-3-yl}-N-(3,5-difluorophenyl)methylsulfonamide,
(R)—N-{1-[(4-chlorophenyl)(pyrid-3-yl)methyl]azetidin-3-yl}-N-(3,5-difluorophenyl)methylsulfonamide,
(S)—N-{1-[(4-chlorophenyl)(pyrid-3-yl)methyl]azetidin-3-yl}-N-(3,5-difluorophenyl)methylsulfonamide,
(RS)—N-{1-[(4-chlorophenyl)(pyrid-4-yl)methyl]azetidin-3-yl}-N-(3,5-difluorophenyl)methylsulfonamide,
(R)—N-{1-[(4-chlorophenyl)(pyrid-4-yl)methyl]azetidin-3-yl}-N-(3,5-difluorophenyl)methylsulfonamide,
(S)—N-{1-[(4-chlorophenyl)(pyrid-4-yl)methyl]azetidin-3-yl}-N-(3,5-difluorophenyl)methylsulfonamide,
(RS)—N-{1-[(4-chlorophenyl)(pyrimidin-5-yl)methyl]azetidin-3-yl}-N-(3,5-difluorophenyl)methylsulfonamide,
(R)—N-{1-[(4-chlorophenyl)(pyrimidin-5-yl)methyl]azetidin-3-yl}-N-(3,5-difluorophenyl)methylsulfonamide,
(S)—N-{1-[(4-chlorophenyl)(pyrimidin-5-yl)methyl]azetidin-3-yl}-N-(3,5-difluorophenyl)methylsulfonamide,
N-{1-[bis(4-chlorophenyl)methyl]azetidin-3-yl}-N-(3,5-difluorophenyl)benzylsulfonamide,

their optical isomers and their pharmaceutically acceptable salts.

The compounds of formula (I) may be prepared using any of the known methods in the art particularly by the procedures as described in U.S. Pat. No. 6,355,631.

In this aspect of the invention, cognition deficits associated with a variety of disorders, particularly, central nervous system's disorders (CNS) can be treated with the compounds of this invention. Examples of CNS disorders include without any limitation, schizophrenia, mood disorders, attention deficit disorders, post-traumatic stress disorders, all kinds of depression, particularly major depressive disorders, bipolar disorders and obsessive compulsive disorders.

In another aspect of this invention there is provided a combination of one or more CB1 receptor antagonists and of one or more antipsychotic agent useful in the treatment of psychiatric disorders. The combination of this invention provides synergistic results in that the combination improves positive and negative symptoms of schizophrenia, weight gain and catalepsy.

Examples of antipsychotic agents that are contemplated to be used in the combination of this invention include all of the known antipsychotic drugs. Specific examples that may be enumerated without any limitation include the following olanzapine (ZYPREXA®), clozapine (CLOZARIL®), haloperidol and haloperidol decanoate (HALDOL®, HALPERON®), loxapine succinate (LOXITANE®), molindone hydrochloride (MOBAN®), pimozide (ORAP®) and risperidone (RISPERDAL®).

There are many ways to show that the compounds of the present invention are useful in treating various diseases as described herein, such as in animal models. For instance, object recognition test is one commonly used animal model to test the efficacy of the compounds in treating diseases involving various cognition impairment. See, for example Ennaceur et al., Behav. Brain Res., 1988, 31, 47-59. The test is based on the spontaneous exploratory activity of the animal and has the characteristics of episodic memory in humans. This memory test is sensitive to ageing (Scali et al., Eur. J. Pharmacol., 1997, 325, 173-180) and to cholinergic dysfunctions (Bartolini et al., Pharm. Biochem. Behav. 1996, 53(2), 277-283) and is based on the differences in the exploration of two objects of fairly similar shape—one familiar, the other new.

Similarly, a working memory performance in a rat hole board model has been used to measure various cognition deficits. The hole board task is a well-known and widely used assay to measure working and reference memory in rodents. This model utilizes a board with 8 holes, each baited with a food reward, thus taking advantage of the rodent's natural propensity to forage for food. In a modified version, it has now been able to assess improvements in working memory performance without the use of amnestic agents. Male Sprague Dawley rats are allowed to find and consume 4 of the 8 rewards and then removed to the home cage for 2 minutes. They are then returned and allowed to find and consume the remaining 4 rewards. Any returns to holes already visited are considered working memory errors. The CB1 antagonists of this invention are found to decrease memory deficit errors significantly.

Cannabinoids can mimic psychotic symptoms in normal people and can precipitate psychotic relapse in vulnerable people. However, recent clinical studies suggest that CB1 antagonists may not be sufficient as a monotherapy to ameliorate positive symptoms in schizophrenia patients. Thus, it has now been contemplated that a co-administration of a CB1 antagonist with an antipsychotic should produce an antipsychotic-like effect, to reverse or diminish the efficacy of a co-administered antipsychotic, and to potentiate the antipsychotic efficacy of a low dose of antipsychotic.

Thus, in one aspect of the treatment of patients affected by schizophrenia using antipsychotic drugs involve side effects such as sedation and illness. For instance, phencyclidine (PCP) and amphetamine-induced hyperlocomotor behavior are useful measures of antipsychotic potential as a significant reversal of that exaggerated activity may indicate antipsychotic potential. PCP and amphetamine are known to effect NMDA and dopaminergic systems which are dysregulated in schizophrenia. Spontaneous locomotion as affected by a test compound is also measured to rule out impact of possible side effects, such as sedation and illness, which could similarly produce a decreased locomotor response on its own.

It has now been found that CB1 antagonist at a suitable dose show no effect on spontaneous locomotion when administered to a patient suffering from schizophrenia. In contrast, the conventional antipsychotic haloperidol at suitable dose shows a significant decrease in spontaneous locomotion due to its sedative nature. It has also been found that the CB1 antagonist of this invention does not reverse hyperlocomotion induced by an antipsychotic such as PCP in a rat model, suggesting, that CB1 antagonists of this invention at these doses, would not be predicted to improve positive symptoms (hallucinations, delusions). In addition, CB1 antagonists of this invention co-administered with differing doses of antipsychotics such as haloperidol or olanzapine yielded comparable results to the effects of the antipsychotics alone. Thus, co-treatment of one or more CB1 antagonists of this invention with an antipsychotic would not be predicted to diminish or enhance the antipsychotic efficacy in patients.

The CB1 antagonists of this invention in combination with an antipsychotic are also useful in improving negative symptoms of schizophrenia. While the most enduring neurobiological hypothesis of schizophrenia is the dopamine (DA) hypothesis positing that the psychotic symptoms of the disorder result from mesolimbic DA hyperactivity (Abi-Dargham A, Gil R, Krystal J, et al (1998): Increased striatal dopamine transmission in schizophrenia: confirmation in a second cohort. Am J Psychiatry 155:761-7; Kapur S, Remington G (2001): Dopamine D(2) receptors and their role in atypical antipsychotic action: still necessary and may even be sufficient. Biol Psychiatry 50:873-83; Weiner I, Joel D (2002) Dopamine in schizophrenia: Dysfunctional information processing in basal ganglia-thalamocortical split circuits. In: Di Chiara G (ed) Handbook of Experimental Pharmacology, vol. 154/II, Dopamine in the CNS II. Springer-Verlag, Berlin, pp 417-472), in recent years increasing role has been given to alteration of glutamatergic transmission, particularly at the NMDA receptor (Goff D C, Coyle J T (2001): The emerging role of glutamate in the pathophysiology and treatment of schizophrenia. Am J Psychiatry 158:1367-77; Javitt D C (1987): Negative schizophrenic symptomatology and the PCP (phencyclidine) model of schizophrenia. Hillside J Clin Psychiatry 9:12-35; Javitt D C (2002): Glycine modulators in schizophrenia. Curr Opin Investig Drugs 3:1067-72; Jentsch J D, Roth R H (1999): The neuropsychopharmacology of phencyclidine: from NMDA receptor hypofunction to the dopamine hypothesis of schizophrenia. Neuropsychopharmacology 20:201-25). A major reason for both hypotheses derives from findings that the administration of both amphetamine and NMDA receptor antagonists such as PCP and MK-801 induce psychosis in healthy humans and exacerbate symptoms in patients. Based on the above, two kinds of animal pharmacological models have evolved to study schizophrenia—amphetamine-based models considered to model the DA abnormality, and NMDAR antagonist-based models thought to model glutamatergic pathology. Because in humans, amphetamine induces only positive symptoms whereas NMDAR antagonists induce also negative and cognitive symptoms of the disorder, amphetamine is considered to model positive symptoms whereas the latter is considered to model negative/cognitive symptoms. This differentiation is supported by the effects of the established and putative antipsychotic drugs (APDs) on amphetamine vs NMDAR induced abnormalities: typically, the former are antagonized by both typical and atypical APDs whereas the latter are antagonized by atypical but not typical APDs. In addition, the NMDAR antagonist abnormalities are sensitive to compounds enhancing NMDAR function via the glycine B site which have been shown to be beneficial against negative symptoms (Halberstadt A L (1995): The phencyclidine-glutamate model of schizophrenia. Clin Neuropharmacol 18:237-49; Javitt D C, Zukin S R (1991): Recent advances in the phencyclidine model of schizophrenia. Am J Psychiatry 148:1301-8; Heresco-Levy U (2003): Glutamatergic neurotransmission modulation and the mechanisms of antipsychotic atypicality. Prog Neuropsychopharmacol Biol Psychiatry 27:1113-23; Heresco-Levy U, Javitt D C (2004): Comparative effects of glycine and -cycloserine on persistent negative symptoms in schizophrenia: a retrospective analysis. Schizophrenia Research 66:89-96; Javitt D C, Coyle J T (2004): Decoding schizophrenia. Sci Am 290:48-55; Krystal J H, D'Souza D C, Mathalon D, Perry E, Belger A, Hoffman R (2003): NMDA receptor antagonist effects, cortical glutamatergic function, and schizophrenia: toward a paradigm shift in medication development. Psychopharmacology (Berl) 169:215-33).

Latent inhibition (LI) is the process whereby pre-exposure to a stimulus retards conditioning to this stimulus when it is subsequently paired with reinforcement, and it has been used extensively to model cognitive impairments in schizophrenia. To date, LI is the only model in which amphetamine and NMDAR antagonists produce different, in fact, opposite, behavioral abnormalities, thus allowing a better screening of potential drugs, because compounds beneficial for positive symptoms and for negative symptoms, produce opposite effects in the model. Briefly, amphetamine disrupts LI in rats and normal humans, and this is paralleled by disrupted LI in acute schizophrenia patients. Amphetamine-induced LI disruption is reversed by both typical and atypical APDs. In contrast, MK-801 produces abnormally persistent LI (LI present under conditions that disrupt it in normal rats) in rats, and this is paralleled by excessive LI in schizophrenia patients with predominantly negative symptoms. Consistent with the pharmacology of NMDAR antagonist models as well as with that of negative symptoms, MK-801 induced persistent LI is reversed by atypical but not typical APDs as well as by glycinergic compounds. As noted above, treatments possessing the capacity to reverse amphetamine and MK-801 induced LI abnormalities, must produce different and in fact opposite actions on the LI phenomenon. Drugs effective in the amphetamine model, restore disrupted LI whereas drugs effective in the MK-801 model disrupt LI. Persistent LI may thus enable an accurate identification of drugs that are effective in reversing NMDAR effects and thus presumably in treating negative symptoms (Gray J A, Feldon J, Rawlins J N P, Hemsley D R, Smith A D (1991): The neuropsychology of schizophrenia. Behav Brain Sci 14:1-20; Moser P C, Hitchcock J M, Lister S, Moran P M (2000): The pharmacology of latent inhibition as an animal model of schizophrenia. Brain Res Rev 33:275-307; Gaisler-Salomon I, Weiner I (2003): Systemic administration of MK-801 produces an abnormally persistent latent inhibition which is reversed by clozapine but not haloperidol. Psychopharmacology (Berl) 166:333-42; Weiner I (2003): The “two-headed” latent inhibition model of schizophrenia: modeling positive and negative symptoms and their treatment. Psychopharmacology, 169:257-297).

Thus as noted above, one measure of negative symptoms of schizophrenia is by measuring the LI, which is measured in a thirst motivated conditioned emotional response (CER) procedure by comparing the suppression of drinking to a tone previously paired with a foot shock in rats that received non-reinforced exposure to the tone prior to conditioning (pre-exposed) and in rats for whom the tone is novel (non-preexposed). The CB1 antagonists of this invention reversed MK801-induced persistent latent inhibition at suitable dosage levels.

Another important side effect of various known psychotic drugs is weight gain. It has now been found that surprisingly the CB1 antagonists of the invention when administered in combination with a psychotic drug controls weight gain in a patient. For instance, olanzapine, a known antipsychotic agent, significantly increases weight gain in a patient. Whereas, a combination of olanzapine and a CB1 antagonist of this invention causes no significant increase in weight gain of a patient.

In another aspect of this invention, it has also been found that catalepsy a side effect normally caused by either a classical antipsychotic agent, such as haloperidol or a atypical antipsychotic agent such as olanzapine can be reduced by co-administration of the CB1 antagonist of this invention with antipsychotic agent. In essence the CB1 antagonists of this invention reduce the extrapyramidal side effects (EPS) elicited by antipsychotic agents when used in combination with such antipsychotic agents.

Of course, clinical trials on humans may also be used to show the usefulness of the compounds of the present invention in treating various disorders as described herein.

Preferably the pharmaceutical compositions of this invention are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. Alternatively, the compositions may be presented in a form suitable for once-weekly or once-monthly administration; for example, an insoluble salt of the active compound, such as the decanoate salt, may be adapted to provide a depot preparation for intramuscular injection. An erodible polymer containing the active ingredient may be envisaged. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Flavored unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The pharmaceutical compositions of this invention can be administered by any of the methods known in the art. In general, the pharmaceutical compositions of this invention can be administered by oral, intramuscular, subcutaneous, rectal, intratracheal, intranasal, intraperitoneal or topical route. The preferred administrations of the pharmaceutical composition of this invention are by oral and intranasal routes. Any of the known methods to administer pharmaceutical compositions by an oral or an intranasal route can be used to administer the composition of this invention.

In the treatment of various disease states as described herein, a suitable dosage level is about 0.01 to 250 mg/kg per day, preferably about 0.05 to 100 mg/kg per day, and especially about 0.05 to 20 mg/kg per day. The compounds may be administered on a regimen of 1 to 4 times per day.

This invention is further illustrated by the following examples which are provided for illustration purposes and in no way limit the scope of the present invention.

Examples 1 and 2 describe typical procedures used for the preparation of a CB1 antagonists in order to prepare the combination of this invention.

### EXAMPLE 1

### N-{1-[Bis(4-chlorophenyl)methyl]azetidin-3-yl}-N-(pyrid-3-yl)methylsulfonamide

The title compound can be prepared by carrying out the preparation in the following way: 0.042 cm³ of phosphorus trichloride is added to a solution of 0.144 g of N-{1-[bis(4-chlorophenyl)methyl]azetidin-3-yl}-N-(1-oxidopyrid-3-yl)methyl-sulfonamide in 5 cm³ of chloroform and then the mixture is heated to the reflux temperature. After stirring for 1 hour 30 minutes, the reaction mixture is allowed to return to normal temperature, 5 cm³ of 0.1 N hydrochloric acid are then added to the mixture, and then the mixture is stirred and separated by settling. The organic phase is diluted with 20 cm³ of chloroform, dried over magnesium sulfate, filtered and then concentrated to dryness under reduced pressure (2.7 kPa). The residue is chromatographed on a column of silica gel (particle size 0.063-0.200 mm, height 9 cm, diameter 1.8 cm), elution being carried out under a pressure of 0.1 bar of argon with a mixture of dichloromethane and of methanol (95/5 by volume) and 15-cm³ fractions being collected. Fractions 2 to 4 are combined and concentrated to dryness under reduced pressure (2.7 kPa). The residue is stirred with 15 cm³ of diethyl ether, the suspension is filtered and the solid is pulled dry and then dried under reduced pressure (2.7 kPa). 35 mg of N-{1-[bis(4-chloro-phenyl)-methyl]azetidin-3-yl}-N-(pyrid-3-yl)methyl-sulfonamide are obtained in the form of a cream solid [¹H N.M.R. spectrum (300 MHz, CDCl₃, δ in ppm): from 2.80 to 2.95 (mt, 2H), 2.87 (s, 3H), 3.51 (split t, J=7 and 1.5 Hz, 2H), 4.18 (s, 1H), 4.65 (mt, 1H), from 7.15 to 7.35 (mt, 8H), 7.37 (broad dd, J=8 and 5 Hz, 1H), 7.64 (reduced d, J=8 Hz, 1H), 8.52 (broad d, J=2 Hz, 1H), 8.61 (broad d, J=5 Hz, 1H)].

### EXAMPLE 2

### Method 1

### N-{1-[Bis(4-chlorophenyl)methyl]azetidin-3-yl}-N-(3,5-difluorophenyl)methylsulfonamide

The title compound can be prepared by carrying out the preparation in the following way: 1.0 g of cesium carbonate is added to a mixture of 1.23 g of 1-[bis(4-chlorophenyl)methyl]azetidin-3-yl}methylsulfonate and of 0.66 g of N-(3,5-difluorophenyl)methylsulfonamide in 25 cm³ of dioxane. After stirring for S hours at the reflux temperature and then for 20 hours at 20° C., 50 cm³ of diethyl ether and 30 cm³ of brine are added to the reaction mixture and then the reaction mixture is stirred and separated by settling. The organic phase is dried over magnesium sulfate, filtered and then concentrated to dryness at 50° C. under reduced pressure (2.7 kPa). The orange oil obtained is chromatographed on a column of silica gel (particle size 0.040-0.063 mm, height 25 cm, diameter 2.0 cm), elution being carried out under a pressure of 0.5 bar of argon with a mixture of cyclohexane and of ethyl acetate (65/35 by volume) and 10-cm³ fractions being collected. Fractions 6 to 10 are combined and concentrated to dryness under reduced pressure (2.7 kPa). The residue is chromatographed on a column of silica gel (particle size 0.040-0.063 mm, height 15 cm, diameter 1.0 cm), elution being carried out under a pressure of 0.5 bar of argon with a mixture of cyclohexane and of ethyl acetate (65/35 by volume) and 5-cm³ fractions being collected. Fraction 7 is concentrated to dryness under reduced pressure (2.7 kPa). 0.11 g of N-{1-[bis(4-chlorophenyl)methyl]-azetidin-3-yl}-N-(3,5-difluorophenyl)methylsulfonamide is obtained in the form of a white powder [¹H N.M.R. spectrum (300 MHz, CDCl₃, δ in ppm): 2.82 (s, 3H), 2.85 (mt, 2H), 3.52 (split t, J=7 and 2 Hz, 2H), 4.22 (s, 1H), 4.47 (mt, 1H), from 6.75 to 6.90 (mt, 3H), from 7.20 to 7.35 (mt, 8H)].

### Method 2

0.78 cm³ of diethyl azodicarboxylate and 1.31 g of triphenylphosphine are added under argon to a solution of 1.41 g of 1-[bis(4-chlorophenyl)methyl]-azetidin-3-ol and of 0.95 g of N-(3,5-difluorophenyl)-methylsulfonamide in 100 cm³ of anhydrous tetrahydrofuran. After stirring for 16 hours at 20° C., 300 cm³ of ethyl acetate are added and the reaction mixture is washed twice with 100 cm³ of water, dried over magnesium sulfate and concentrated to dryness under reduced pressure (2.7 kPa). The residue is chromatographed on a column of silica gel (particle size 0.20-0.063 mm, height 50 cm, diameter 4 cm), elution being carried out under a pressure of 0.6 bar of argon with a mixture of cyclohexane and of ethyl acetate (75/25 by volume) and 125-cm³ fractions being collected. Fractions 6 to 12 are combined and concentrated to dryness under reduced pressure (2.7 kPa). 1.8 g of a solid are obtained, which solid is dissolved under hot conditions in an ethyl acetate/diisopropyl ether mixture (15/2 by volume), cooled and diluted with 100 cm³ of pentane to initiate crystallization. After filtration and drying, 1.0 g of N-{1-[bis(4-chlorophenyl)methyl]azetidin-3-yl}-N-(3,5-difluorophenyl)methylsulfonamide is obtained in the form of white crystals melting at 154° C.

N-(3,5-Difluorophenyl)methylsulfonamide can be prepared by carrying out the preparation in the following way: 2.0 cm³ of methylsulfonyl chloride, 3.8 cm³ of triethylamine and 20 mg of 4-dimethylamino-pyridine are slowly added to a solution of 3.5 g of 3,5-difluoroaniline in 75 cm³ of dichloromethane. After stirring for 20 hours at 20° C., the reaction mixture, to which 20 cm³ of dichloromethane and 20 cm³ of water are added, is stirred and then separated by settling. The organic phase is dried over magnesium sulfate, filtered and then concentrated to dryness under reduced pressure (2.7 kPa). The residue is chromatographed on a column of silica gel (particle size 0.063-0.200 mm, height 20 cm, diameter 2.0 cm), elution being carried out under a pressure of 0.1 bar of argon with dichloromethane and 25-cm³ fractions being collected. Fractions 14 to 20 are combined and concentrated to dryness under reduced pressure (2.7 kPa). 0.66 g of N-(3,5-difluorophenyl)methylsulfonamide is obtained in the form of a white powder.

1-[Bis(4-chlorophenyl)methyl]azetidin-3-yl methylsulfonate can be prepared by carrying out the preparation in the following way: 3.5 cm³ of methylsulfonyl chloride are added under argon over 10 minutes to a solution of 12 g of 1-[bis(4-chloro-phenyl)methyl]azetidin-3-ol in 200 cm³ of dichloromethane, then the mixture is cooled to +5° C. and 3.8 cm³ of pyridine are added in over 10 minutes. After stirring for 30 minutes at +5° C. and then for 20 hours at 20° C., the reaction mixture is diluted with 100 cm³ of water and 100 cm³ of dichloromethane. The mixture, filtered first, is separated by settling. The organic phase is washed with water and then dried over magnesium sulfate, filtered and concentrated to dryness under reduced pressure (2.7 kPa). The oil obtained is chromatographed on a column of silica gel (particle size 0.063-0.200 mm, height 40 cm, diameter 3.0 cm), elution being carried out under a pressure of 0.5 bar of argon with a mixture of cyclohexane and of ethyl acetate (70/30 by volume) and 100-cm³ fractions being collected. Fractions 4 to 15 are combined and concentrated to dryness under reduced pressure (2.7 kPa). 6.8 g of 1-[bis(4-chlorophenyl)methyl]-azetidin-3-yl methylsulfonate are obtained in the form of a yellow oil.

1-[Bis(4-chlorophenyl)methyl]azetidin-3-ol can be prepared according to the procedure described by Katritzky A. R. et al., J. Heterocycl. Chem., 271 (1994), starting from 35.5 g of [bis(4-chlorophenyl)methyl]amine hydrochloride and 11.0 cm³ of epichlorohydrin. 9.0 g of 1-[bis(4-chlorophenyl)methyl]azetidin-3-ol are isolated.

[Bis(4-chlorophenyl)methyl]amine hydrochloride can be prepared according to the method described by Grisar M. et al., J. Med. Chem., 885 (1973).

### EXAMPLE 3

### Holeboard Test

This test shows the efficacy of the CB1 antagonists of this invention when administered alone or in combination with an antipsychotic agent.

Animals: Male Sprague Dawley rats (Charles River) were housed on a 12 hour light/dark cycle, with lights on at 06:00. Rats were maintained at 80% of their normal body weight, with average starting weights at 200-220 grams. Rats were acclimated to the testing chamber (Med-Associates, Inc. hole board in a ventilated, sound-attenuating cubicle) for four 10-minute trials over a two-day period 24 hours prior to drug treatments. The testing chamber contains eight holes, each of which is baited with a food reward (cocoa puff). Procedure: Each experiment was carried over two-three days, with a 3 day (experiment 1), 4 day (experiment 2), and 3 day (experiment 3) washout in between. Thirty two animals were used for each experiment, with each animal pseudo-randomly assigned to treatment groups such that each animal received two of the four-five treatments, with an even distribution of all possible treatment-treatment combinations. There were a total of 16 animals per treatment group. On test days, rats were injected intraperitoneally (i.p.) with N-{1-[bis(4-chlorophenyl)methyl]azetidin-3-yl}-N-(3,5-difluorophenyl)methylsulfonamide (Example 2) (Exp 1: 0.3, 1, or 3 mg/kg; Exp 2: 1, 3 or 10 mg/kg) or vehicle (distilled water with 1% tween). For Exp 3, rats were injected i.p. with risperidone followed by Example 2 (Exp 3: 0.010, 0.10, or 1.0 mg/kg with 3 mg/kg Example 2) or vehicle (0.9% NaCl with 1% tween). The rats were then placed in the test chamber 60 minutes later. After consuming 4 food rewards, the rats were removed from the chamber for 2 minutes and returned to their home cage. They were then returned to the chamber and allowed to retrieve and finish the remaining four rewards, or for a total of 10 minutes. Animals that did not retrieve all 8 rewards within the 10 minute period were excluded from the study. The number of visits to holes that they have already visited were noted.
Drugs: Doses of Example 2 were 0.3, 1 and 3 mg/kg (experiment 1) and 1, 3 and 10 mg/kg (experiment 2). Example 2 was put into suspension with distilled water (exp 1 & 2) or 0.9% NaCl (exp 3), with the addition of tween 80. Doses of risperidone (antipsychotic, Sigma) were 0.010, 0.10, and 1.0 mg/kg (experiment 3). risperidone was solubilized in 0.9% NaCl with the addition of 1% tween 80,

In Experiment 1, there was a significant effect of treatment on the number of working memory errors following the 2 minute delay. The 3 mg/kg dose but not the 0.3 or 1 mg/kg doses of Example 2 caused a significant decrease in the number of working memory errors compared to vehicle treated animals (p<0.05). In Experiment 2, there was a significant effect of treatment on the number of working errors following the 2 minute delay. The 3 mg/kg and 10 mg/kg, but not 1 mg/kg dose of Example 2 caused a significant decrease in the number of working memory errors compared to vehicle treated animals (p<0.05). In Experiment 3, there was a significant effect of treatment on the number of working errors following the 2 minute delay with Example 2 alone and in combination with risperidone. 3 mg/kg of Example 2 alone and in combination with 0.10 mg/kg of risperidone, but not 0.010, or 1.0 mg/kg risperidone, caused a significant decrease in the number of working memory errors compared to vehicle treated animals (p<0.05). Example 2 alone or in combination with risperidone did not effect the latency to complete the task in either experiment.

This test demonstrates that Example 2 significantly decreased the number of visits to holes previously visited, indicating an improvement in working memory performance in this model. The minimum effective dose for this effect was 3 mg/kg. Further, 3 mg/kg of Example 2 improved working memory performance in the presence of 0.1 mg/kg risperidone. These data support a potential utility of Example 2 as a treatment for cognitive deficits associated with schizophrenia.

### EXAMPLE 4

Test for Positive Symptoms of Schizophrenia

Animals: Male CD-1 mice (Charles River Laboratories) weighing 20-30 g were used. Male Sprague-Dawley rats (Charles River Laboratories) weighing 250-433 g were used. The animals were housed under standard laboratory conditions as outlined in the NIH Guide for Care and Use of Laboratory Animals. They were maintained on a 12:12 light/dark cycle with tap water and Lab Diet rodent chow ad libitum. Mice were acclimatized to the experimental room for 60 min prior to injections.
Procedure: A standard automated locomotion assay was employed (see, for example: R. Christopher Pierce and Peter Kalivas. (1997) Locomotor Behavior. In: Current Protocols in Neuroscience, Volume 3, 8.1.1-8.1.8. G. P. Taylor, Editor. New York: John Wiley & Sons, Inc.). Horizontal activity was measured by beam breaks of photocells lining the chamber exterior to the activity boxes. The activity was measured for 60 min during spontaneous locomotion or 90 min for the PCP or amphetamine-induced assays. Example 2 was administered per os (po) with a 1 hr pre-treatment. In co-administration experiments either haloperidol or olanzapine was administered intraperitoneally (ip) with a 30 min pre-treatment. PCP or amphetamine was administered ip or subcutaneous (sc), respectively, with no pre-treatment. When pre-treatment time elapsed for each rodent, the activity cage was transferred from its holding rack and placed into its own locomotion chamber. Independent start time is possible with the recording of activity commencing almost immediately. The computer automatically times out each chamber individually when the session has ended.
Drugs: Doses of Example 2 for mice were 0.3, 1, 3, and 10 mg/kg, p.o. The lowest dose was not tested in spontaneous locomotion for rats. The three highest doses of Example 2 were tested against PCP-induced and amphetamine-induced locomotion in mice and rats, respectively. The conventional antipsychotic haloperidol was used in co-administration with Example 2 (1, 3, and 10 mg/kg) at the doses of 0.1 and 0.2 mg/kg in mice to reverse PCP-induced locomotion. Haloperidol was co-administered at the dose of 0.3 mg/kg in rats to reverse amphetamine-induced locomotion. The atypical antipsychotic olanzapine was used in co-administration with Example 2 (1, 3, and 10 mg/kg) at the doses of 0.03 and 0.3 mg/kg in mice to reverse PCP-induced locomotion. Olanzapine was co-administered at the doses of 1 and 3 mg/kg in rats to reverse amphetamine-induced locomotion.

Example 2 was suspended via homogenization in 60% labrasol/40% labrafil for all mouse experiments and most rat experiments. For the rat spontaneous and 1 mg/kg olanzapine tests, Example 2 was suspended in sterile water with a drop of tween 80. Haloperidol was dissolved in distilled water via dilution of a 5 mg/ml stock solution in distilled water. Olanzapine had the addition of a drop of acetic acid (mice) or a drop of HCl (rats) prior to the addition of distilled water. Phencyclidine and amphetamine were dissolved in distilled water.

Example 2 administered alone at doses of 0.3, 1, 3, or 10 mg/kg did not significantly alter spontaneous locomotion in either mice or rats. Example 2 alone at doses of 1, 3, and 10 mg/kg demonstrated no significant reversal of locomotor hyperactivity induced by PCP in mice or by d-AMPH in rats. This was in contrast with the highly significant reversal shown by haloperidol (0.3 mg/kg). Co-treatment of Example 2 with haloperidol at two doses (0.1 and 0.2 mg/kg) in mice and one dose (0.3 mg/kg) in rats yielded the same effects whether or not Example 2 was present or absent. Similarly, co-treatment of Example 2 with olanzapine at two doses (0.03 and 0.3 mg/kg) in mice and two doses (1 and 3 mg/kg) in rats yielded the same effects whether or not Example 2 was present or absent. In virtually every treatment group the level of significance remained the same whether Example 2 was present in combination with the antipsychotic or not. No significant difference was found for olanzapine or haloperidol alone versus any of the combinations tested.

This example demonstrates that the CB1 antagonists of this invention have no effect on spontaneous locomotion in either mice or rats. This is beneficial in that certain side effects, such as the potential sedation exhibited by haloperidol could be ruled out. The lack of impact of Example 2 on PCP- or amphetamine-induced hyperlocomotion indicates that as a monotherapy, no effects on positive symptoms would be predicted. Lastly, the co-treatment of Example 2 with the conventional antipsychotic haloperidol or the atypical antipsychotic olanzapine yielded comparable results to the administration of Example 2 alone. It is therefore suggested that Example 2 would not diminish the antipsychotic effects of these widely prescribed antipsychotics yet provide additional benefits as disclosed herein.

### EXAMPLE 5

### Test for Negative Symptoms of Schizophrenia

This Example 5 uses latent inhibition (LI) as a measure of negative symptoms of schizophrenia. LI was measured in a thirst motivated conditioned emotional response (CER) procedure by comparing the suppression of drinking to a tone previously paired with a foot shock in rats that received non-reinforced exposure to the tone prior to conditioning (pre-exposed) and in rats for whom the tone was novel (non-preexposed). Example 2 reversed MK801-induced persistent latent inhibition at 1, 3 and 10 mg/kg i.p. Apparatus and Procedure: Rats were tested in Campden Instruments rodent test chambers with a retractable bottle. When the bottle was not present, the hole was covered by a metal lid. Licks were detected by a Campden Instruments drinkometer. The preexposed to-be-conditioned stimulus was a 10 sec, 80 dB, 2.8 kHz tone produced by a Sonalert module. Shock was supplied through the floor by a Campden Instruments shock generator and shock scrambler set at 0.5 mA and 1 sec duration. Equipment programming and data recording were computer controlled.

LI was measured in a thirst motivated conditioned emotional response (CER) procedure by comparing the suppression of drinking to a tone previously paired with a foot shock in rats that received nonreinforced exposure to the tone prior to conditioning (preexposed) and in rats for whom the tone was novel (nonpreexposed). Parameters that do not produce LI in no-drug controls, 40 preexposures and 5 conditioning trials, were used, because persistent LI can be manifested only with such parameters.

Prior to the beginning of each LI experiment, rats were handled for about 2 min daily for 5 days. A 23 h water restriction schedule was initiated simultaneously with handling and continued throughout the experiment. On the next 5 days, rats were trained to drink in the experimental chamber for 20 min/day. Water in the test apparatus was given in addition to the daily ration of 1 h given in the home cages. The LI procedure was conducted on days 11-14 and consisted of the following stages:

Preexposure: With the bottle removed, the preexposed (PE) rats received 40 tone presentations with an inter-stimulus interval of 50 sec. The nonpreexposed (NPE) rats were confined to the chamber for an identical period of time without receiving the tone. Conditioning—With the bottle removed, each rat received 5 tone-shock pairings given 5 min apart. Shock immediately followed tone termination. The first tone-shock pairing was given 5 min after the start of the session. After the last pairing, rats were left in the experimental chamber for an additional 5 min.

Rebaseline: Rats were given a 15 min drinking session as in initial training. Data of rats that failed to complete 600 licks were dropped from the analysis.

Test: Each rat was placed in the chamber and allowed to drink from the bottle. When the rat completed 75 licks the tone was presented for 5 min. The following times were recorded: Time to first lick, time to complete licks 1-50, time to complete licks 51-75 (before tone onset) and time to complete licks 76-100 (after tone onset). Times to complete licks 76-100 were logarithmically transformed to allow parametric analysis of variance. Longer log times indicate stronger suppression of drinking. LI is defined as significantly shorter log times to complete licks 76-100 of the preexposed as compared to nonpreexposed rats. In addition, number of licks made during the presentation of the tone, was recorded in 5 blocks of 30 sec.

Drugs: Drugs were administered intraperitoneally. MK-801 (dizocilpine; Merck Research Laboratories, USA) was diluted in saline and administered at a dose of 0.05 mg/kg (Gaisler-Salomon I, Weiner I (2003): Systemic administration of MK-801 produces an abnormally persistent latent inhibition which is reversed by clozapine but not haloperidol. Psychopharmacology (Berl) 166:333-42), at a volume of 1 ml/kg 30 minutes before conditioning. Example 2 was dissolved in 1-2 drops of tween 80 solution (polyoxyethylene sorbitan monooleate; Sigma, Israel) and diluted in dH₂O, and administered in a volume of 1 ml/kg at doses of either 1, 3 or 10 mg/kg (D1, D2 and D3, respectively) 60 minutes prior to pre-exposure and conditioning stages. Glycine (Sigma, Israel) was diluted with vehicle and administered 30 minutes prior to the conditioning stage at a dose of 0.8 g/kg, in volume of 3 ml/kg. No-drug controls received the corresponding vehicle.
Results: The experiment included 191 rats (run in 4 replications) in 20 groups in a 2×2×5 design with main factors of preexposure (PE, NPE), treatment (vehicle, MK-801), and pretreatment (vehicle, D1, D2, D3, glycine). Data of 15 rats were dropped from the analysis. n per group was 8-10, except for the saline-glycine-NPE group (n=7). The 20 experimental groups did not differ in their times to complete licks 51-75 before tone onset (all p's>0.5; overall mean A period=8.32 sec). The data show that, vehicle-injected rats did not show LI, whereas MK-801-treated rats showed LI in spite of extended conditioning. MK-801-induced abnormally persistent LI was reversed by D1, D2, D3, and glycine, so that MK-801-treated rats did not show LI like controls.

### EXAMPLE 6

### Antipsychotic Induced Weight Gain

This Example demonstrates the efficacy of the CB 1 antagonists of this invention in controlling the weight gain induced by antipsychotics, such as olanzapine.

Animals: Female Wistar rats on high fat diet were used in this Example.
Drugs: Doses of olanzapine were 3 mg/kg intraperitoneally (i.p.) in co-administration with doses of Example 2 at 1, 3, and 10 mg/kg i.p. and a dose of Example 2 alone at 10 mg/kg i.p. was used for comparison, and saline solution is used as a control.
Results: A two way analysis of variance (ANOVA) revealed a significant effect of time and treatment for weight increase and food consumption. Olanzapine significantly caused an increase in weight gain vs. saline controls. Increase was significant in 5 days and lasted to the end of the study. Co administration of Example 2 caused a dose dependent attenuation of the weight gain elicited by olanzapine. Co treatment of 10 mg/kg of Example 2i.p. with olanzapine was not significant from saline controls. Example 2 alone at a dose of 10 mg/kg i.p. had no significant effect on weight vs. saline. Food consumption data was too variable to make concrete conclusions. Overall all the treatment groups with olanzapine appeared to consume greater quantities of food than saline.

### EXAMPLE 7

### Antipsychotic Induced Catalepsy

Animals: Male Sprague-Dawley rats (Charles River Laboratories) weighing 267-457 g were used. The animals were housed under standard laboratory conditions as outlined in the NIH Guide for Care and Use of Laboratory Animals. They were maintained on a 12:12 light/dark cycle with tap water and Lab Diet rodent chow ad libitum. Rats were acclimatized to the experimental room for 60 min prior to injections.
Procedure: The test for catalepsy consists of placing an individual animal in a white translucent plastic box (26×20×15 cm) with a wooden dowel mounted horizontally 10 cm from the floor and 4 cm from one end of the box. The floor is covered with approximately 1 cm of bedding material. Test animals are transferred from the vivarium in their home cages to the experimental room and are allowed to acclimatize for 60 mins. Five animals are kept in a cage. The animals are transferred into another cage after treatment. Test animals were administered with either vehicle or Example 2 orally. After a period of 30 mins, the animals received either 1 mg/kg of haloperidol or 10 mg/kg of olanzapine intraperitoneally. 30 mins after the second treatment, animals were placed individually in the white translucent plastic boxes and tested for catalepsy following a one minute acclimatization period. A group of five animals are tested at a time. Each treatment group consists of 10 animals.

At the end of the one minute acclimation period, each animal is gently grasped around the shoulders and under the forepaws, and gently placed on the wooden dowel. The amount of time each rat spends with at least one forepaw on the bar determined for a maximum period of 180 seconds. This is repeated three times.

Drugs: Doses of Example 2 tested were 1, 3, and 10 mg/kg. Haloperidol was used at the dose of 1 mg/kg. Olanzapine was administered at the dose of 10 mg/kg.

Example 2 was suspended via homogenization in 60% labrasol/40% labrafil with two drops of Tween 80 added. Haloperidol was dissolved in distilled water via dilution of a 5 mg/ml stock solution in distilled water. Olanzapine was dissolved in three drops of HCl prior to the addition of distilled water to full volume.

Results: Haloperidol significantly induces catalepsy in rats at doses of 1 and 3 mg/kg compared to vehicle treated animals with ED₅₀ of 0.64(0.33-1.26) mg/kg. Olanzapine, on the other hand only induces catalepsy at higher dose of 10 mg/kg with ED₅₀ of 9.34(6.82-12.78) mg/kg.

Example 2 administered alone at dose of 10 mg/kg did not significantly induce catalepsy in rats. Example 2 at a dose of 10 mg/kg significantly reversed haloperidol-induced catalepsy. Similarly, Example 2 significantly reversed olanzapine-induced catalepsy at 3 mg/kg and 10 mg/kg.

This Example demonstrates that Example 2 did not induce catalepsy in rats. Further, Example 2 significantly reduced catalepsy induced by either the typical antipsychotic haloperidol or the atypical antipsychotic olanzapine. This data suggests the potential utility of CB1 antagonists of this invention in reducing extrapyramidal side effects associated with antipsychotic therapy.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A method of treatment of cognition deficits in a patient suffering from schizophrenia comprising administering to said patient a therapeutically effective amount of a CB1 receptor antagonist of formula (I): 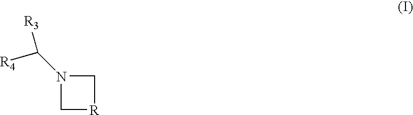
in which
either A:
R represents a CR₁R₂, C═C(R₅)SO₂R₆ or C═C(R₇)SO₂alk radical,
either R₁ represents a hydrogen atom and R₂ represents a —C(R₈)(R₉)(R₁₀), —C(R₈)(R₁₁)(R₁₂), —CO—NR₁₃R₁₄, —CH₂—CO—NR₁₃R₁₄, —CH₂—CO—R₆, —CO—R₆, —CO-cycloalkyl, —SO—R₆, —SO₂—R₆, —C(OH)(R₁₂)(R₆), —C(OH)(R₆)(alkyl), —C(═NOalk)R₆, —C(═NO—CH₂—CH═CH₂)R₆, —CH₂—CH(R₆)NR₃₁R₃₂, —CH₂—C(═NOalk)R₆, —CH(R₆)NR₃₁R₃₂, —CH(R₆)NHSO₂alk, —CH(R₆)NHCONHalk or —CH(R₆)NHCOalk radical,
or R₁ represents an alkyl, NH—R₁₅, cyano, —S-alk-NR₁₆R₁₇, —CH₂—NR₁₈R₁₉ or —NR₂₀R₂₁ radical and R₂ represents a —C(R₈)(R₁₁)(R₁₂) radical,
R₃ and R₄, which are identical or different, represent either an alkyl or cycloalkyl radical, or an aromatic radical chosen from phenyl, naphthyl or indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₂₂R₂₃, —CO—NH—NR₂₄R₂₅, alkylsulfanyl, alkylsulfinyl, alkylsulfonyl, alkylsulfanylalkyl, alkylsulfinylalkyl, alkylsulfonylalkyl, hydroxyalkyl or -alk-NR₂₄R₂₅; or a heteroaromatic radical chosen from the benzofuryl, benzothiazolyl, benzothienyl, benzoxazolyl, chromanyl, 2,3-dihydroxybenzofuryl, 2,3-dihydrobenzothienyl, furyl, imidazolyl, isochromanyl, isoquinolyl, pyrrolyl, pyridyl, pyrimidinyl, quinolyl, 1,2,3,4-tetrahydro-isoquinolyl, thiazolyl and thienyl rings, it being possible for these heteroaromatic radicals to be unsubstituted or substituted by one or more halogen, alkyl, alkoxy, hydroxyl, trifluoromethyl, trifluoromethoxy, cyano, —COOH, —COOalk, —CO—NH—NR₂₄R₂₅, —CONR₂₂R₂₃, -alk-NR₂₄R₂₅, alkylsulfanyl, alkylsulfinyl, alkylsulfonyl, alkylsulfanylalkyl, alkylsulfinylalkyl, alkylsulfonylalkyl or hydroxyalkyl,
R₅ represents a hydrogen atom or an alkyl radical,
R₆ represents an Ar₁ or Het₁ radical,
R₇ represents a cycloalkyl, heterocycloalkyl or heterocyclenyl radical optionally substituted by a —CSO-phenyl radical,
R₈ represents a hydrogen atom or an alkyl radical,
R₉ represents a —CO—NR₂₆R₂₇, —COOH, —COOalk, —CH₂OH, —NH—CO—NH-alk, —CH₂—NHR₂₈ or —NHCOOalk radical,
R₁₀ represents an Ar₁ or Het₁ radical,
R₁₁ represents an —SO₂-alk, —SO₂—Ar₁ or —SO₂-Het₁ radical,
R₁₂ represents a hydrogen atom or an Ar₁ or Het₁ radical,
R₁₃ represents a hydrogen atom or an alkyl radical,
R₁₄ represents an Ar₁, Het₁, -alk-Ar₁ or -alk-Het₁ radical,
R₁₅ represents an alkyl, cycloalkyl or -alk-NR₂₉R₃₀ radical,
R₁₆ and R₁₇, which are identical or different, represent a hydrogen atom or an alkyl radical or else R₁₆ and R₁₇ form, together with the nitrogen atom to which they are attached, a saturated or unsaturated and mono- or bicyclic heterocycle having 3 to 10 ring members and optionally comprising one or more other heteroatoms chosen from oxygen, sulfur and nitrogen and optionally substituted by one or more alkyl radicals,
R₁₈ represents a hydrogen atom or an alkyl radical,
R₁₉ represents a hydrogen atom or an alkyl, cycloalkyl, cycloalkylalkyl, cycloalkylcarbonyl, —SO₂alk, —CO—NHalk or —COOalk radical,
or else, R₁₈ and R₁₉ form, with the nitrogen atom to which they are attached, a saturated or unsaturated and mono- or bicyclic heterocycle having 3 to 10 ring members and optionally comprising one or more heteroatoms chosen from oxygen, sulfur and nitrogen and optionally substituted by one or more alkyl radicals,
—NR₂₀R₂₁ represents a saturated or unsaturated monocyclic heterocycle having 3 to 8 ring members and optionally comprising another heteroatom chosen from oxygen, nitrogen and sulfur,
R₂₂ and R₂₃, which are identical or different, represent a hydrogen atom or an alkyl radical or else R₂₂ and R₂₃ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one more alkyl radicals,
R₂₄ and R₂₅, which are identical or different, represent a hydrogen atom or an alkyl, —COOalk, cycloalkyl, alkylcycloalkyl, -alk-O-alk or hydroxyalkyl radical or else R₂₄ and R₂₅ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂ radicals,
R₂₆ and R₂₇, which are identical or different, represent a hydrogen atom or an alkyl, hydroxyalkyl, cycloalkyl, cycloalkylalkyl, -alk-COOalk, -alk-Ar₁, alk-Het₁, Het₁ or -alk-N(alk)₂ radical, R₂₆ and R₂₇ can also form, with the nitrogen atom to which they are attached, a saturated or unsaturated and mono- or bicyclic heterocycle having 3 to 10 ring members and optionally comprising one or more heteroatoms chosen from oxygen, sulfur and nitrogen and optionally substituted by one or more alkyl, alkoxy or halogen radicals,
R₂₈ represents a —CH₂-alk, benzyl, —SO₂alk, —CONHalk, —COalk, cycloalkylalkylcarbonyl, cycloalkylcarbonyl or —CO—(CH₂)ₙOH radical,
n is equal to 1, 2, or 3,
R₂₉ and R₃₀, which are identical or different, represent a hydrogen atom or an alkyl radical or else R₂₉ and R₃₀ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl radicals,
R₃₁ and R₃₂, which are identical or different, represent a hydrogen atom or an alkyl, Ar₁ or -alk-Ar₁ radical or else R₃₁ and R₃₂ form, together with the nitrogen atom to which they are attached, a heterocycle chosen from aziridinyl, azetidinyl, pyrrolidinyl and piperidinyl,
Ar₁ represents a phenyl or naphthyl radical optionally substituted by one or more substituents chosen from halogen, alkyl, alkoxy, —CO-alk, cyano, —COOH, —COOalk, —CONR₂₂R₂₃, —CO—NH—NR₂₄R₂₅, alkylsulfanyl, alkylsulfinyl, alkylsulfonyl, alkylsulfanylalkyl, alkylsulfinylalkyl, alkylsulfonylalkyl, hydroxyalkyl, -alk-NR₂₄R₂₅, —NR₂₄R₂₅, alkylthioalkyl, formyl, hydroxyl, CF₃, OCF₃, Het₁, O-alk-NH-cycloalkyl or SO₂NH₂,
Het₁ represents a saturated or unsaturated and mono- or bicyclic heterocycle having 3 to 10 ring members and comprising one or more heteroatoms chosen from oxygen, sulfur and nitrogen and optionally substituted by one or more halogen, alkyl, alkoxy, alkoxycarbonyl, —CONR₂₂R₂₃, hydroxyl, hydroxyalkyl, oxo or SO₂NH₂,
or B:
R represents a CHR₃₃ radical,
R₃₃ represents an —NHCOR₃₄ or —N(R₃₅)—Y—R₃₆ radical,
Y is CO or SO₂,
R₃ and R₄, which are identical or different, represent either an aromatic radical chosen from phenyl, naphthyl and indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₃₇R₃₈, —CO—NH—NR₃₉R₄₀, alkylsulfanyl, alkylsulfinyl, alkylsulfonyl, alkylsulfanylalkyl, alkylsulfinylalkyl, alkylsulfonylalkyl, hydroxyalkyl or -alk-NR₃₇R₃₈; or a heteroaromatic radical chosen from the benzofuryl, benzothiazolyl, benzothienyl, benzoxazolyl, chromanyl, 2,3-dihydrobenzofuryl, 2,3-dihydrobenzothienyl, pyrimidinyl, furyl, imidazolyl, isochromanyl, isoquinolyl, pyrrolyl, pyridyl, quinolyl, 1,2,3,4-tetrahydroisoquinolyl, thiazolyl and thienyl rings, it being possible for these heteroaromatic radicals to be unsubstituted or substituted by a halogen, alkyl, alkoxy, hydroxyl, trifluoromethyl, trifluoromethoxy, cyano, —COOH, —COOalk, —CO—NH—NR₃₉R₄₀, —CONR₃₇R₃₈, -alk-NR₃₉R₄₀, alkylsulfanyl, alkylsulfinyl, alkylsulfonyl, alkylsulfanylalkyl, alkylsulfinylalkyl, alkylsulfonylalkyl or hydroxyalkyl,
R₃₄ represents an -alk-SO₂—R₄₁ radical, an -alk-SO₂—CH═CH—R₄₁ radical, a Het₂ radical substituted by —SO₂—R₄, or a phenyl radical substituted by —SO₂—R₄₁ or -alk-SO₂—R₄₁,
R₃₅ represents a hydrogen atom or an alkyl radical,
R₃₆ represents a phenylalkyl, Het₂ or Ar₂ radical,
R₃₇ and R₃₈, which are identical or different, represent a hydrogen atom or an alkyl radical or else R₃₇ and R₃₈ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
R₃₉ and R₄₀, which are identical or different, represent a hydrogen atom or an alkyl, —COOalk, cycloalkyl, alkylcycloalkyl, -alk-O-alk or hydroxyalkyl radical or else R₃₉ and R₄₀ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂,
R₄₁ represents an alkyl, Ar₂ or Het₂ radical,
Ar₂ represents a phenyl, naphthyl or indenyl radical, these radicals optionally being substituted by one or more halogen, alkyl, alkoxy, cyano, —CO-alk, —COOH, —COOalk, —CONR₄₂R₄₃, —CO—NH—NR₄₄R₄₅, alkylsulfanyl, alkylsulfinyl, alkylsulfonyl, -alk-NR₄₄R₄₅, —NR₄₄R₄₅, alkylthioalkyl, formyl, hydroxyl, hydroxyalkyl, Het₂, —O-alk-NH-cycloalkyl, OCF₃, CF₃, —NH—CO-alk, —SO₂NH₂, —HN—COCH₃, —NH—COOalk or Het₂ or else on two adjacent carbon atoms by a dioxymethylene,
Het₂ represents a saturated or unsaturated and mono- or bicyclic heterocycle having 3 to 10 ring members and comprising one or more heteroatoms chosen from oxygen, sulfur and nitrogen optionally substituted by one or more alkyl, alkoxy, vinyl, halogen, alkoxycarbonyl, oxo, hydroxyl, OCF₃ or CF₃, the nitrogenous heterocycles optionally being in their N-oxidized form,
R₄₂ and R₄₃, which are identical or different, represent a hydrogen atom or an alkyl radical or else R₄₂ and R₄₃ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl radicals,
R₄₄ and R₄₅, which are identical or different, represent a hydrogen atom or an alkyl, —COOalk, cycloalkyl, alkylcycloalkyl, -alk-O-alk or hydroxyalkyl radical or else R₄₄ and R₄₅ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂ radicals,
or C:
R represents a CHR₄₆ radical,
R₄₆ represents an —N(R₄₇)R₄₈, —N(R₄₇)—CO—R₄₈ or —N(R₄₇)—SO₂R₄₉ radical,
R₃ and R₄, which are identical or different, represent either an aromatic radical chosen from phenyl, naphthyl and indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₅₀R₅₁, —CO—NH—NR₅₂R₅₃, alkylsulfanyl, alkylsulfinyl, alkylsulfonyl, alkylsulfanylalkyl, alkylsulfinylalkyl, alkylsulfonylalkyl, hydroxyalkyl or -alk-NR₇R₈ radicals; or a heteroaromatic radical chosen from the benzofuryl, benzothiazolyl, benzothienyl, benzoxazolyl, chromanyl, 2,3-dihydrobenzofuryl, 2,3-dihydrobenzothienyl, furyl, imidazolyl, isochromanyl, isoquinolyl, pyrrolyl, pyridyl, pyrimidyl, quinolyl, 1,2,3,4-tetrahydroisoquinolyl, thiazolyl and thienyl rings, it being possible for these heteroaromatic radicals to be unsubstituted or substituted by a halogen, alkyl, alkoxy, hydroxyl, trifluoromethyl, trifluoromethoxy, cyano, —COOH, —COOalk, —CO—NH—NR₅₂R₅₃, —CONR₅₀R₅₁, -alk-NR₅₂R₅₃, alkylsulfanyl, alkylsulfinyl, alkylsulfonyl, alkylsulfanylalkyl, alkylsulfinylalkyl, alkylsulfonylalkyl or hydroxyalkyl radical,
R₄₇ represents a —C(R₅₄)(R₅₅)-Het₃, -Het₃, —C(R₅₄)(R₅₅)—Ar₃, Ar₃, cycloalkyl or norbornyl radical,
R₄₈ represents a hydrogen atom or a hydroxyalkyl radical, -alk-COOalk radical, -alk-CONR₅₀R₅₁ radical, -alk-NR₅₀R₅₁ radical, alkoxy radical, Ar₃ radical, Het₃ radical, —CH₂Ar₃ radical, —CH₂Het₃ radical or alkyl radical optionally substituted with one or more halogen,
R₄₉ represents a hydroxyalkyl radical, -alk-COOalk radical, -alk-CONR₅₀R₅₁ radical, -alk-NR₅₀R₅₁ radical, alkoxy radical, Ar₃ radical, Het₃ radical, —CH₂Ar₃ radical, —CH₂Het₃ radical or alkyl radical optionally substituted with one or more halogen,
R₅₀ and R₅₁, which are identical or different, represent a hydrogen atom or an alkyl radical or else R₅₀ and R₅₁ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
R₅₂ and R₅₃, which are identical or different, represent a hydrogen atom or an alkyl, —COOalk, cycloalkyl, alkylcycloalkyl, -alk-O-alk or hydroxyalkyl radical or else R₅₂ and R₅₃ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂,
R₅₄ represents a hydrogen atom or a hydroxyalkyl radical, -alk-COOalk radical, -alk-CONR₅₀R₅₁ radical, -alk-NR₅₀R₅₁ radical, alkoxyalkyl radical, Ar₃ radical, Het₃ radical, —CH₂Ar₃ radical, —CH₂Het₃ radical or alkyl radical optionally substituted with one or more halogen,
R₅₅ represents a hydrogen atom or a hydroxyalkyl radical, -alk-COOalk radical, -alk-CONR₅₀R₅₁ radical, -alk-NR₅₀R₅₁ radical, alkoxyalkyl radical or alkyl radical optionally substituted with one or more halogen,
or else R₅₄ and R₅₅ form, together with the carbon atom to which they are attached, a saturated mono- or bicyclic ring having 3 to 10 ring members optionally comprising another heteroatom chosen from oxygen, sulfur and nitrogen and optionally being substituted by one or more alkyl,
Ar₃ represents a phenyl, naphthyl or indenyl radical, these various radicals optionally being substituted by one or more halogen, alkyl, alkoxy, —CO-alk, cyano, —COOH, —COOalk, —CONR₅₆R₅₇, —CO—NH—NR₅₈R₅₉, alkylsulfanyl, alkylsulfinyl, alkylsulfonyl, -alk-NR₅₈R₅₉, —NR₅₈R₅₉, alkylthioalkyl, formyl, CF₃, OCF₃, Het₃, —O-alk-NH-cycloalkyl, SO₂NH₂, hydroxyl, hydroxyalkyl, —NHCOalk or —NHCOOalk or on 2 adjacent carbon atoms by dioxymethylene,
Het₃ represents a saturated or unsaturated and mono- or bicyclic heterocycle having 3 to 10 ring members and comprising one or more heteroatoms chosen from oxygen, sulfur and nitrogen optionally substituted by one or more alkyl, alkoxy, halogen, alkoxycarbonyl, oxo or hydroxyl, the nitrogenous heterocycles optionally being in their N-oxidized form,
R₅₆ and R₅₇, which are identical or different, represent a hydrogen atom or an alkyl radical or else R₅₆ and R₅₇ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
R₅₈ and R₅₉, which are identical or different, represent a hydrogen atom or an alkyl radical or else R₅₈ and R₅₉ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
alk represents an alkyl or alkylene radical, and
the alkyl, alkylene and alkoxy radicals feature either straight or branched chains and comprise 1 to 6 carbon atoms, the cycloalkyl radicals comprise 3 to 10 carbon atoms and the heterocycloalkyl and heterocyclenyl radicals comprise 3 to 10 carbon atoms, or
an optical isomer of said CB1 antagonist or a pharmaceutically acceptable salt thereof.

2. The method as set forth in claim 1, wherein the CB1 antagonist is selected from the group consisting of:
(RS)-1-[bis(4-chlorophenyl)methyl)]-3-[(3,5-difluoro-phenyl)(methylsulfonyl)methyl]azetidine,
(R)-1-[bis(4-chlorophenyl)methyl)]-3-[(3,5-difluoro-phenyl)(methylsulfonyl)methyl]azetidine,
(S)-1-[bis(4-chlorophenyl)methyl)]-3-[(3,5-difluoro-phenyl)(methylsulfonyl)methyl]azetidine,
(RS)-1-[bis(4-chlorophenyl)methyl)]-3-[(pyrid-3-yl)-(methylsulfonyl)methyl]azetidine,
(R)-1-[bis(4-chlorophenyl)methyl)]-3-[(pyrid-3-yl)-(methylsulfonyl)methyl]azetidine,
(S)-1-[bis(4-chlorophenyl)methyl)]-3-[(pyrid-3-yl)-(methylsulfonyl)methyl]azetidine,
(RS)-1-[bis(3-fluorophenyl)methyl]-3-[(3,5-difluorophenyl)(methylsulfonyl)methyl]azetidine,
(R)-1-[bis(3-fluorophenyl)methyl]-3-[(3,5-difluorophenyl)(methylsulfonyl)methyl]azetidine,
(S)-1-[bis(3-fluorophenyl)methyl]-3-[(3,5-difluorophenyl)(methylsulfonyl)methyl]azetidine,
1-[bis(4-chlorophenyl)methyl]-3-(RS)-{[3-(azetidin-1-yl)phenyl](methylsulfonyl)methyl}azetidine,
1-[bis(4-chlorophenyl)methyl]-3-(R)-{[3-(azetidin-1-yl)phenyl](methylsulfonyl)methyl}azetidine,
1-[bis(4-chlorophenyl)methyl]-3-(S)-{[3-(azetidin-1-yl)phenyl](methylsulfonyl)methyl}azetidine,
(RS)-1-[3-({1-[bis(4-chlorophenyl)methyl]azetidin-3-yl}(methylsulfonyl)methyl)phenyl]pyrrolidine,
(R)-1-[3-({1-[bis(4-chlorophenyl)methyl]azetidin-3-yl}(methylsulfonyl)methyl)phenyl]pyrrolidine,
(S)-1-[3-({1-[bis(4-chlorophenyl)methyl]azetidin-3-yl}(methylsulfonyl)methyl)phenyl]pyrrolidine,
(RS)—N-[3-({1-[bis(4-chlorophenyl)methyl]azetidin-3-yl}(methylsulfonyl)methyl)phenyl]-N-methylamine,
(R)—N-[3-({1-[bis(4-chlorophenyl)methyl]azetidin-3-yl}(methylsulfonyl)methyl)phenyl]-N-methylamine,
(S)—N-[3-({1-[bis(4-chlorophenyl)methyl]azetidin-3-yl}(methylsulfonyl)methyl)phenyl]-N-methylamine,
(RS)-1-[bis(4-chlorophenyl)methyl]-3-[(3,5-bis(trifluoro-methyl)phenyl)(methylsulfonyl)methyl]azetidine,
(R)-1-[bis(4-chlorophenyl)methyl]-3-[(3,5-bis(trifluoro-methyl)phenyl)(methylsulfonyl)methyl]azetidine,
(S)-1-[bis(4-chlorophenyl)methyl]-3-[(3,5-bis(trifluoro-methyl)phenyl)(methylsulfonyl)methyl]azetidine,
1-[bis(4-chlorophenyl)methyl]-3-(phenylsulfonyl-methyl)azetidine,
(RS)-1-[bis(4-chlorophenyl)methyl]-3-[(3,5-difluoro-phenyl)(methylsulfonyl)methyl]-3-methylazetidine,
(R)-1-[bis(4-chlorophenyl)methyl]-3-[(3,5-difluorophenyl)methylsulfonylmethyl]-3-methylazetidine,
(S)-1-[bis(4-chlorophenyl)methyl]-3-[(3,5-difluoro-phenyl)(methylsulfonyl)methyl]-3-methylazetidine,
(RS)-2-{1-[bis(4-chlorophenyl)methyl]azetidin-3-yl}-2-(3,5-difluorophenyl)-N-cyclohexylacetamide,
(R)-2-{1-[bis(4-chlorophenyl)methyl]azetidin-3-yl}-2-(3,5-difluorophenyl)-N-cyclohexylacetamide,
(S)-2-{1-[bis(4-chlorophenyl)methyl]azetidin-3-yl}-2-(3,5-difluorophenyl)-N-cyclohexylacetamide,
(RS)-2-{1-[bis(4-chlorophenyl)methyl]azetidin-3-yl}-2-(3,5-difluorophenyl)-N-isobutylacetamide,
(R)-2-{1-[bis(4-chlorophenyl)methyl]azetidin-3-yl}-2-(3,5-difluorophenyl)-N-isobutylacetamide,
(S)-2-{1-[bis(4-chlorophenyl)methyl]azetidin-3-yl}-2-(3,5-difluorophenyl)-N-isobutylacetamide,
(RS)-2-{1-[bis(4-chlorophenyl)methyl]azetidin-3-yl}-2-(3,5-difluorophenyl)-N-cyclopropylmethylacetamide,
(R)-2-{1-[bis(4-chlorophenyl)methyl]azetidin-3-yl}-2-(3,5-difluorophenyl)-N-isopropylacetamide,
(S)-2-{1-[bis(4-chlorophenyl)methyl]azetidin-3-yl}-2-(3,5-difluorophenyl)-N-cyclopropylmethylacetamide,
(RS)-2-{1-[bis(4-chlorophenyl)methyl]azetidin-3-yl}-2-(3,5-difluorophenyl)-N-isopropylacetamide,
(R)-2-{1-[bis(4-chlorophenyl)methyl]azetidin-3-yl}-2-(3,5-difluorophenyl)-N-isopropylacetamide,
(S)-2-{1-[bis(4-chlorophenyl)methyl]azetidin-3-yl}-2-(3,5-difluorophenyl)-N-isopropylacetamide,
(RS)-1-[bis(4-chlorophenyl)methyl]-3-[1-(3,5-difluorophenyl)-1-(methylsulfonyl)ethyl]azetidine,
(R)-1-[bis(4-chlorophenyl)methyl]-3-[1-(3,5-difluorophenyl)-1-(methylsulfonyl)ethyl]azetidine,
(S)-1-[bis(4-chlorophenyl)methyl]-3-[1-(3,5-difluorophenyl)-1-(methylsulfonyl)ethyl]azetidine,
(RS)-1-[bis(4-fluorophenyl)methyl]-3-[(3,5-difluoro-phenyl)(methylsulfonyl)methyl]azetidine,
(R)-1-[bis(4-fluorophenyl)methyl]-3-[(3,5-difluoro-phenyl)(methylsulfonyl)methyl]azetidine,
(S)-1-[bis(4-fluorophenyl)methyl]-3-[(3,5-difluoro-phenyl)(methylsulfonyl)methyl]azetidine,
(RS)-{1-[(3-pyridyl)(4-chlorophenyl)methyl]-3-[(3,5-difluoro-phenyl)(methylsulfonyl)methyl]azetidine,
(SS)-{1-[(3-pyridyl)(4-chlorophenyl)methyl]-3-[(3,5-difluoro-phenyl)(methylsulfonyl)methyl]azetidine,
(RR)-{1-[(3-pyridyl)(4-chlorophenyl)methyl]-3-[(3,5-difluoro-phenyl)(methylsulfonyl)methyl]azetidine,
(SR)-{1-[(3-pyridyl)(4-chlorophenyl)methyl]-3-[(3,5-difluoro-phenyl)(methylsulfonyl)methyl]azetidine,
(RS)-{1-[(4-pyridyl)(4-chlorophenyl)methyl]-3-[(3,5-difluoro-phenyl)(methylsulfonyl)methyl]azetidine,
(SS)-{1-[(4-pyridyl)(4-chlorophenyl)methyl]-3-[(3,5-difluoro-phenyl)(methylsulfonyl)methyl]azetidine,
(RR)-{1-[(4-pyridyl)(4-chlorophenyl)methyl]-3-[(3,5-difluoro-phenyl)(methylsulfonyl)methyl]azetidine,
(SR)-{1-[(4-pyridyl)(4-chlorophenyl)methyl]-3-[(3,5-difluoro-phenyl)(methylsulfonyl)methyl]azetidine,
(RS)-5-((4-chlorophenyl) {3-[(3,5-difluorophenyl)(methylsulfonyl)methyl]azetidin-1-yl}methyl)pyrimidine,
(SR)-5-((4-chlorophenyl){3-[(3,5-difluorophenyl)(methylsulfonyl)methyl]azetidin-1-yl}methyl)pyrimidine,
(RR)-5-((4-chlorophenyl) {3-[(3,5-difluorophenyl)(methylsulfonyl)methyl]azetidin-1-yl}methyl)pyrimidine,
(SS)-5-((4-chlorophenyl) {3-[(3,5-difluorophenyl)(methylsulfonyl)methyl]azetidin-1-yl}methyl)pyrimidine,
(SS)-{1-[(2-chloropyrid-5-yl)(4-chlorophenyl)methyl]-3-[(3,5-difluorophenyl)(methylsulfonyl)methyl]azetidine,
(RR)-{1-[(2-chloropyrid-5-yl)(4-chlorophenyl)methyl]-3-[(3,5-difluorophenyl)(methylsulfonyl)methyl]azetidine,
(RS)-{1-[(2-chloropyrid-5-yl)(4-chlorophenyl)methyl]-3-[(3,5-difluorophenyl)(methylsulfonyl)methyl]azetidine,
(SR)-{1-[(2-chloropyrid-5-yl)(4-chlorophenyl)methyl]-3-[(3,5-difluorophenyl)(methylsulfonyl)methyl]azetidine,
N-{1-[bis(4-chlorophenyl)methyl]azetidin-3-yl}thien-2-ylsulfonamide,
N-{1-[bis(4-chlorophenyl)methyl]azetidin-3-yl}-4-methoxyphenylsulfonamide,
N-[4-(N-{1-[bis(4-chlorophenyl)methyl]azetidin-3-yl}sulfamoyl)phenyl]acetamide,
N-{1-[bis(4-chlorophenyl)methyl]azetidin-3-yl}-4-methylphenylsulfonamide,
N-{1-[bis(4-chlorophenyl)methyl]azetidin-3-yl}-3,4-dimethoxyphenylsulfonamide,
N-{1-[bis(4-chlorophenyl)methyl]azetidin-3-yl}-3-fluorophenylsulfonamide,
N-{1-[bis(4-chlorophenyl)methyl]azetidin-3-yl}-3,4-dichlorophenylsulfonamide,
N-{1-[bis(4-chlorophenyl)methyl]azetidin-3-yl}-3-cyanophenylsulfonamide,
N-{1-[bis(4-chlorophenyl)methyl]azetidin-3-yl}-2,5-dimethoxyphenylsulfonamide,
N-{1-[bis(4-chlorophenyl)methyl]azetidin-3-yl}-3-trifluoromethylphenylsulfonamide,
N-{1-[bis(4-chlorophenyl)methyl]azetidin-3-yl}naphth-2-ylsulfonamide,
N-{1-[bis(4-chlorophenyl)methyl]azetidin-3-yl}naphth-1-ylsulfonamide,
N-{1-[bis(4-chlorophenyl)methyl]azetidin-3-yl}-3,4-difluorophenylsulfonamide,
N-{1-[bis(4-chlorophenyl)methyl]azetidin-3-yl}-1-methyl-1H-imidazol-4-ylsulfonamide,
N-[4-(N-{1-[bis(4-chlorophenyl)methyl]azetidin-3-yl}sulfamoyl)-2-chlorophenyl]acetamide,
N-{1-[bis(4-chlorophenyl)methyl]azetidin-3-yl}pyrid-3-ylsulfonamide,
N-{1-[bis(4-chlorophenyl)methyl]azetidin-3-yl}-4-fluorophenylsulfonamide,
N-{1-[bis(4-chlorophenyl)methyl]azetidin-3-yl}quinol-8-ylsulfonamide,
N-{1-[bis(4-chlorophenyl)methyl]azetidin-3-yl}phenylsulfonamide,
N-{1-[bis(4-chlorophenyl)methyl]azetidin-3-yl}(phenylmethyl)sulfonamide,
N-{1-[bis(4-chlorophenyl)methyl]azetidin-3-yl}-3,5-difluorophenylsulfonamide,
N-{1-[bis(4-chlorophenyl)methyl]azetidin-3-yl}pyrid-2-ylsulfonamide,
N-{1-[bis(4-chlorophenyl)methyl]azetidin-3-yl}-(3-fluoro-5-pyrrolidin-1-ylphenyl)sulfonamide,
N-{1-[bis(4-chlorophenyl)methyl]azetidin-3-yl}-N-methyl-4-fluorophenylsulfonamide,
N-{1-[bis(4-chlorophenyl)methyl]azetidin-3-yl}-N-methylquinol-8-ylsulfonamide,
N-{1-[bis(4-chlorophenyl)methyl]azetidin-3-yl}-N-methylphenylsulfonamide,
N-{1-[bis(4-chlorophenyl)methyl]azetidin-3-yl}-N-methyl(phenylmethyl)sulfonamide,
N-{1-[bis(4-chlorophenyl)methyl]azetidin-3-yl}-3-sulfamoylphenylsulfonamide,
2-benzenesulfonyl-N-{1-[bis(4-chlorophenyl)methyl]azetidin-3-yl}acetamide,
N-{1-[bis(4-chlorophenyl)methyl]azetidin-3-yl}-2-(toluene-4-sulfonyl)acetamide,
(3-chloro-4-(methylsulfonyl)thiophene-2-carboxy) {1-[bis(4-chlorophenyl)methyl]azetidin-3-yl}amide,
N-{1-[bis(4-chlorophenyl)methyl]azetidin-3-yl}-3-(2-phenylethylenesulfonyl)propionamide,
N-{1-[bis(4-chlorophenyl)methyl]azetidin-3-yl}-4-(methylsulfonyl)benzamide,
(5-(methylsulfonyl)thiophene-2-carboxy)-{1-[bis(4-chlorophenyl)methyl]azetidin-3-yl}amide,
(5-(methylsulfonyl)-3-methyl-4-vinylthiophene-2-carboxy) {1-[bis(4-chlorophenyl)methyl]azetidin-3-yl}amide,
(RS)—N-{1-[{4-chlorophenyl)(pyridin-3-yl)methyl]azetidin-3-yl}-3,5-difluorobenzenesulfonamide,
(RS)—N-{1-[{4-chlorophenyl)(pyrimidin-5-yl)methyl]azetidin-3-yl}-3,5-difluorobenzenesulfonamide,
N-{1-[bis{4-chlorophenyl)methyl]azetidin-3-yl}-N-(6-chloropyrid-2-yl)methylsulfonamide,
N-{1-[bis{4-chlorophenyl)methyl]azetidin-3-yl}-N-(6-ethylpyrid-2-yl)methylsulfonamide,
N-{1-[bis{4-chlorophenyl)methyl]azetidin-3-yl}-N-(quinol-6-yl)methylsulfonamide,
N-{1-[bis{4-chlorophenyl)methyl]azetidin-3-yl}-N-(quinol-5-yl)methylsulfonamide,
N-{1-[bis{4-chlorophenyl)methyl]azetidin-3-yl}-N-(isoquinol-5-yl)methylsulfonamide,
N-{1-[bis{4-chlorophenyl)methyl]azetidin-3-yl}-N-(pyrid-3-yl)methylsulfonamide,
N-{1-[bis{4-chlorophenyl)methyl]azetidin-3-yl}-N-(1-oxidopyrid-3-yl)methylsulfonamide,
N-((1R,2S,4S)bicyclo[2.2.1]hept-2-yl)-N-{1-[bis(4-chlorophenyl)methyl]azetidin-3-yl}methylsulfonamide,
N-((1R,2R,4S)bicyclo[2.2.1]hept-2-yl)-N-{1-[bis(4-chlorophenyl)methyl]azetidin-3-yl}methylsulfonamide,
N-{1-[bis(4-chlorophenyl)methyl]azetidin-3-yl}-N-(3,5-difluorophenyl)methylsulfonamide,
N-{1-[bis(4-chlorophenyl)methyl]azetidin-3-yl}-N-(thiazol-2-yl)methylsulfonamide,
N-{1-[bis(4-chlorophenyl)methyl]azetidin-3-yl}-N-(3-methoxyphenyl)methylsulfonamide,
N-{1-[bis(4-chlorophenyl)methyl]azetidin-3-yl}-N-(3-(hydroxyphenyl)methylsulfonamide,
N-{1-[bis(4-chlorophenyl)methyl]azetidin-3-yl}-N-(3-(hydroxymethyl)phenyl)methylsulfonamide,
ethyl N-{1-[bis(4-chlorophenyl)methyl]azetidin-3-yl}-N-(methylsulfonyl)-3-aminobenzoate
N-{1-[bis(4-chlorophenyl)methyl]azetidin-3-yl}-N-(1-isobutylpiperid-4-yl)methylsulfonamide,
N-benzyl-N-{1-{bis(4-chlorophenyl)methyl]azetidin-3-yl}amine
N-{1-[bis(4-chlorophenyl)methyl]azetidin-3-yl}-N-(3,5-difluorobenzyl)amine,
N-{1-[bis(4-chlorophenyl)methyl]azetidin-3-yl}-N-(3,5-difluorobenzyl)methylsulfonamide,
N-{1-[bis(4-chlorophenyl)methyl]azetidin-3-yl}-N-(pyrid-3-ylmethyl)methylsulfonamide,
N-{1-[bis(4-fluorophenyl)methyl]azetidin-3-yl}-N-(3,5-difluorophenyl)methylsulfonamide,
(RS)—N-{1-[(4-chlorophenyl)(pyrid-3-yl)methyl]azetidin-3-yl}-N-(3,5-difluorophenyl)methylsulfonamide,
(R)—N-{1-[(4-chlorophenyl)(pyrid-3-yl)methyl]azetidin-3-yl}-N-(3,5-difluorophenyl)methylsulfonamide,
(S)—N-{1-[(4-chlorophenyl)(pyrid-3-yl)methyl]azetidin-3-yl}-N-(3,5-difluorophenyl)methylsulfonamide,
(RS)—N-{1-[(4-chlorophenyl)(pyrid-4-yl)methyl]azetidin-3-yl}-N-(3,5-difluorophenyl)methylsulfonamide,
(R)—N-{1-[(4-chlorophenyl)(pyrid-4-yl)methyl]azetidin-3-yl}-N-(3,5-difluorophenyl)methylsulfonamide,
(S)—N-{1-[(4-chlorophenyl)(pyrid-4-yl)methyl]azetidin-3-yl}-N-(3,5-difluorophenyl)methylsulfonamide,
(RS)—N-{1-[(4-chlorophenyl)(pyrimidin-5-yl)methyl]azetidin-3-yl}-N-(3,5-difluorophenyl)methylsulfonamide,
(R)—N-{1-[(4-chlorophenyl)(pyrimidin-5-yl)methyl]azetidin-3-yl}-N-(3,5-difluorophenyl)methylsulfonamide,
(S)—N-{1-[(4-chlorophenyl)(pyrimidin-5-yl)methyl]azetidin-3-yl}-N-(3,5-difluorophenyl)methylsulfonamide, and
N-{1-[bis(4-chlorophenyl)methyl]azetidin-3-yl}-N-(3,5-difluorophenyl)benzylsulfonamide,
or an optical isomer or a pharmaceutically acceptable salt thereof.

3. The method as set forth in claim 1, wherein the CB1 antagonist is selected from the group consisting of:
N-{1-[bis(4-chlorophenyl)methyl]azetidin-3-yl}-N-(pyrid-3-yl)methylsulfonamide, and
N-{1-[bis(4-chlorophenyl)methyl]azetidin-3-yl}-N-(3,5-difluorophenyl)methylsulfonamide
or an optical isomer or a pharmaceutically acceptable salt thereof.

4. A method for the treatment of a psychiatric disorder in a patient comprising administering to said patient a combination of CB1 receptor antagonist with one or more antipsychotic agents, wherein said CB1 antagonist is of formula (I): 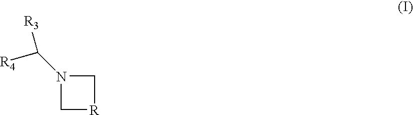
in which
either A:
R represents a CR₁R₂, C═C(R₅)SO₂R₆ or C═C(R₇)SO₂alk radical,
either R₁ represents a hydrogen atom and R₂ represents a —C(R₈)(R₉)(R₁₀), —C(R₈)(R₁₁)(R₁₂), —CO—NR₁₃R₁₄, —CH₂—CO—NR₁₃R₁₄, —CH₂—CO—R₆, —CO—R₆, —CO-cycloalkyl, —SO—R₆, —SO₂—R₆, —C(OH)(R₁₂)(R₆), —C(OH)(R₆)(alkyl), —C(═NOalk)R₆, —C(═NO—CH₂—CH═CH₂)R₆, —CH₂—CH(R₆)NR₃₁R₃₂, —CH₂—C(═NOalk)R₆, —CH(R₆)NR₃₁R₃₂, —CH(R₆)NHSO₂alk, —CH(R₆)NHCONHalk or —CH(R₆)NHCOalk radical,
or R₁ represents an alkyl, NH—R₁₅, cyano, —S-alk-NR₁₆R₁₇, —CH₂—NR₁₈R₁₉ or —NR₂₀R₂₁ radical and R₂ represents a —C(R₈)(R₁₁)(R₁₂) radical,
R₃ and R₄, which are identical or different, represent either an alkyl or cycloalkyl radical, or an aromatic radical chosen from phenyl, naphthyl or indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₂₂R₂₃, —CO—NH—NR₂₄R₂₅, alkylsulfanyl, alkylsulfinyl, alkylsulfonyl, alkylsulfanylalkyl, alkylsulfinylalkyl, alkylsulfonylalkyl, hydroxyalkyl or -alk-NR₂₄R₂₅; or a heteroaromatic radical chosen from the benzofuryl, benzothiazolyl, benzothienyl, benzoxazolyl, chromanyl, 2,3-dihydroxybenzofuryl, 2,3-dihydrobenzothienyl, furyl, imidazolyl, isochromanyl, isoquinolyl, pyrrolyl, pyridyl, pyrimidinyl, quinolyl, 1,2,3,4-tetrahydro-isoquinolyl, thiazolyl and thienyl rings, it being possible for these heteroaromatic radicals to be unsubstituted or substituted by one or more halogen, alkyl, alkoxy, hydroxyl, trifluoromethyl, trifluoromethoxy, cyano, —COOH, —COOalk, —CO—NH—NR₂₄R₂₅, —CONR₂₂R₂₃, -alk-NR₂₄R₂₅, alkylsulfanyl, alkylsulfinyl, alkylsulfonyl, alkylsulfanylalkyl, alkylsulfinylalkyl, alkylsulfonylalkyl or hydroxyalkyl,
R₅ represents a hydrogen atom or an alkyl radical,
R₆ represents an Ar₁ or Het₁ radical,
R₇ represents a cycloalkyl, heterocycloalkyl or heterocyclenyl radical optionally substituted by a —CSO-phenyl radical,
R₈ represents a hydrogen atom or an alkyl radical,
R₉ represents a —CO—NR₂₆R₂₇, —COOH, —COOalk, —CH₂OH, —NH—CO—NH-alk, —CH₂—NHR₂₈ or —NHCOOalk radical,
R₁₀ represents an Ar₁ or Het₁ radical,
R₁₁ represents an —SO₂-alk, —SO₂—Ar₁ or —SO₂-Het₁ radical,
R₁₂ represents a hydrogen atom or an Ar₁ or Het₁ radical,
R₁₃ represents a hydrogen atom or an alkyl radical,
R₁₄ represents an Ar₁, Het₁, -alk-Ar₁ or -alk-Het₁ radical,
R₁₅ represents an alkyl, cycloalkyl or -alk-NR₂₉R₃₀ radical,
R₁₆ and R₁₇, which are identical or different, represent a hydrogen atom or an alkyl radical or else R₁₆ and R₁₇ form, together with the nitrogen atom to which they are attached, a saturated or unsaturated and mono- or bicyclic heterocycle having 3 to 10 ring members and optionally comprising one or more other heteroatoms chosen from oxygen, sulfur and nitrogen and optionally substituted by one or more alkyl radicals,
R₁₈ represents a hydrogen atom or an alkyl radical,
R₁₉ represents a hydrogen atom or an alkyl, cycloalkyl, cycloalkylalkyl, cycloalkylcarbonyl, —SO₂alk, —CO—NiHalk or —COOalk radical,
or else, R₁₈ and R₁₉ form, with the nitrogen atom to which they are attached, a saturated or unsaturated and mono- or bicyclic heterocycle having 3 to 10 ring members and optionally comprising one or more heteroatoms chosen from oxygen, sulfur and nitrogen and optionally substituted by one or more alkyl radicals,
—NR₂₀R₂₁ represents a saturated or unsaturated monocyclic heterocycle having 3 to 8 ring members and optionally comprising another heteroatom chosen from oxygen, nitrogen and sulfur,
R₂₂ and R₂₃, which are identical or different, represent a hydrogen atom or an alkyl radical or else R₂₂ and R₂₃ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one more alkyl radicals,
R₂₄ and R₂₅, which are identical or different, represent a hydrogen atom or an alkyl, —COOalk, cycloalkyl, alkylcycloalkyl, -alk-O-alk or hydroxyalkyl radical or else R₂₄ and R₂₅ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂ radicals,
R₂₆ and R₂₇, which are identical or different, represent a hydrogen atom or an alkyl, hydroxyalkyl, cycloalkyl, cycloalkylalkyl, -alk-COOalk, -alk-Ar₁, alk-Het₁, Het₁ or -alk-N(alk)₂ radical, R₂₆ and R₂₇ can also form, with the nitrogen atom to which they are attached, a saturated or unsaturated and mono- or bicyclic heterocycle having 3 to 10 ring members and optionally comprising one or more heteroatoms chosen from oxygen, sulfur and nitrogen and optionally substituted by one or more alkyl, alkoxy or halogen radicals,
R₂₈ represents a —CH₂-alk, benzyl, —SO₂alk, —CONHalk, —COalk, cycloalkylalkylcarbonyl, cycloalkylcarbonyl or —CO—(CH₂)ₙOH radical,
n is equal to 1, 2, or 3,
R₂₉ and R₃₀, which are identical or different, represent a hydrogen atom or an alkyl radical or else R₂₉ and R₃₀ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl radicals,
R₃₁, and R₃₂, which are identical or different, represent a hydrogen atom or an alkyl, Ar₁ or -alk-Ar₁ radical or else R₃₁ and R₃₂ form, together with the nitrogen atom to which they are attached, a heterocycle chosen from aziridinyl, azetidinyl, pyrrolidinyl and piperidinyl,
Ar₁ represents a phenyl or naphthyl radical optionally substituted by one or more substituents chosen from halogen, alkyl, alkoxy, —CO-alk, cyano, —COOH, —COOalk, —CONR₂₂R₂₃, —CO—NH—NR₂₄R₂₅, alkylsulfanyl, alkylsulfinyl, alkylsulfonyl, alkylsulfanylalkyl, alkylsulfinylalkyl, alkylsulfonylalkyl, hydroxyalkyl, -alk-NR₂₄R₂₅, —NR₂₄R₂₅, alkylthioalkyl, formyl, hydroxyl, CF₃, OCF₃, Het₁, O-alk-NH-cycloalkyl or SO₂NH₂,
Het₁ represents a saturated or unsaturated and mono- or bicyclic heterocycle having 3 to 10 ring members and comprising one or more heteroatoms chosen from oxygen, sulfur and nitrogen and optionally substituted by one or more halogen, alkyl, alkoxy, alkoxycarbonyl, —CONR₂₂R₂₃, hydroxyl, hydroxyalkyl, oxo or SO₂NH₂,
or B:
R represents a CHR₃₃ radical,
R₃₃ represents an —NHCOR₃₄ or —N(R₃₅)—Y—R₃₆ radical,
Y is CO or SO₂,
R₃ and R₄, which are identical or different, represent either an aromatic radical chosen from phenyl, naphthyl and indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₃₇R₃₈, —CO—NH—NR₃₉R₄₀, alkylsulfanyl, alkylsulfinyl, alkylsulfonyl, alkylsulfanylalkyl, alkylsulfinylalkyl, alkylsulfonylalkyl, hydroxyalkyl or -alk-NR₃₇R₃₈; or a heteroaromatic radical chosen from the benzofuryl, benzothiazolyl, benzothienyl, benzoxazolyl, chromanyl, 2,3-dihydrobenzofuryl, 2,3-dihydrobenzothienyl, pyrimidinyl, furyl, imidazolyl, isochromanyl, isoquinolyl, pyrrolyl, pyridyl, quinolyl, 1,2,3,4-tetrahydroisoquinolyl, thiazolyl and thienyl rings, it being possible for these heteroaromatic radicals to be unsubstituted or substituted by a halogen, alkyl, alkoxy, hydroxyl, trifluoromethyl, trifluoromethoxy, cyano, —COOH, —COOalk, —CO—NH—NR₃₉R₄₀, —CONR₃₇R₃₈, -alk-NR₃₉R₄₀, alkylsulfanyl, alkylsulfinyl, alkylsulfonyl, alkylsulfanylalkyl, alkylsulfinylalkyl, alkylsulfonylalkyl or hydroxyalkyl,
R₃₄ represents an -alk-SO₂—R₄₁ radical, an -alk-SO₂—CH═CH—R₄₁ radical, a Het₂ radical substituted by —SO₂—R₄₁ or a phenyl radical substituted by —SO₂—R₄₁ or -alk-SO₂—R₄₁,
R₃₅ represents a hydrogen atom or an alkyl radical,
R₃₆ represents a phenylalkyl, Het₂ or Ar₂ radical,
R₃₇ and R₃₈, which are identical or different, represent a hydrogen atom or an alkyl radical or else R₃₇ and R₃₈ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
R₃₉ and R₄₀, which are identical or different, represent a hydrogen atom or an alkyl, —COOalk, cycloalkyl, alkylcycloalkyl, -alk-O-alk or hydroxyalkyl radical or else R₃₉ and R₄₀ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂,
R₄₁ represents an alkyl, Ar₂ or Het₂ radical,
Ar₂ represents a phenyl, naphthyl or indenyl radical, these radicals optionally being substituted by one or more halogen, alkyl, alkoxy, cyano, —CO-alk, —COOH, —COOalk, —CONR₄₂R₄₃, —CO—NH—NR₄₄R₄₅, alkylsulfanyl, alkylsulfinyl, alkylsulfonyl, -alk-R₅, —NR₄₄R₄₅, alkylthioalkyl, formyl, hydroxyl, hydroxyalkyl, Het₂, —O-alk-NH-cycloalkyl, OCF₃, CF₃, —NH—CO-alk, —SO₂NH₂, —HN—COCH₃, —NH—COOalk or Het₂ or else on two adjacent carbon atoms by a dioxymethylene,
Het₂ represents a saturated or unsaturated and mono- or bicyclic heterocycle having 3 to 10 ring members and comprising one or more heteroatoms chosen from oxygen, sulfur and nitrogen optionally substituted by one or more alkyl, alkoxy, vinyl, halogen, alkoxycarbonyl, oxo, hydroxyl, OCF₃ or CF₃, the nitrogenous heterocycles optionally being in their N-oxidized form,
R₄₂ and R₄₃, which are identical or different, represent a hydrogen atom or an alkyl radical or else R₄₂ and R₄₃ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl radicals,
R₄₄ and R₄₅, which are identical or different, represent a hydrogen atom or an alkyl, —COOalk, cycloalkyl, alkylcycloalkyl, -alk-O-alk or hydroxyalkyl radical or else R₄₄ and R₄₅ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂ radicals,
or C:
R represents a CHR₄₆ radical,
R₄₆ represents an —N(R₄₇)R₄₈, —N(R₄₇)—CO—R₄₈ or —N(R₄₇)—SO₂R₄₉ radical,
R₃ and R₄, which are identical or different, represent either an aromatic radical chosen from phenyl, naphthyl and indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₅₀R₅₁, —CO—NH—NR₅₂R₅₃, alkylsulfanyl, alkylsulfinyl, alkylsulfonyl, alkylsulfanylalkyl, alkylsulfinylalkyl, alkylsulfonylalkyl, hydroxyalkyl or -alk-NR₇R₈ radicals; or a heteroaromatic radical chosen from the benzofuryl, benzothiazolyl, benzothienyl, benzoxazolyl, chromanyl, 2,3-dihydrobenzofuryl, 2,3-dihydrobenzothienyl, furyl, imidazolyl, isochromanyl, isoquinolyl, pyrrolyl, pyridyl, pyrimidyl, quinolyl, 1,2,3,4-tetrahydroisoquinolyl, thiazolyl and thienyl rings, it being possible for these heteroaromatic radicals to be unsubstituted or substituted by a halogen, alkyl, alkoxy, hydroxyl, trifluoromethyl, trifluoromethoxy, cyano, —COOH, —COOalk, —CO—NH—NR₅₂R₅₃, —CONR₅₀R₅₁, -alk-NR₅₂R₅₃, alkylsulfanyl, alkylsulfinyl, alkylsulfonyl, alkylsulfanylalkyl, alkylsulfinylalkyl, alkylsulfonylalkyl or hydroxyalkyl radical,
R₄₇ represents a —C(R₅₄)(R₅₅)-Het₃, -Het₃, —C(R₅₄)(R₅₅)—Ar₃, Ar₃, cycloalkyl or norbornyl radical,
R₄₈ represents a hydrogen atom or a hydroxyalkyl radical, -alk-COOalk radical, -alk-CONR₅₀R₅₁ radical, -alk-NR₅₀R₅₁ radical, alkoxy radical, Ar₃ radical, Het₃ radical, —CH₂Ar₃ radical, —CH₂Het₃ radical or alkyl radical optionally substituted with one or more halogen,
R₄₉ represents a hydroxyalkyl radical, -alk-COOalk radical, -alk-CONR₅₀R₅₁ radical, -alk-NR₅₀R₅₁ radical, alkoxy radical, Ar₃ radical, Het₃ radical, —CH₂Ar₃ radical, —CH₂Het₃ radical or alkyl radical optionally substituted with one or more halogen,
R₅₀ and R₅₁, which are identical or different, represent a hydrogen atom or an alkyl radical or else R₅₀ and R₅₁ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
R₅₂ and R₅₃, which are identical or different, represent a hydrogen atom or an alkyl, —COOalk, cycloalkyl, alkylcycloalkyl, -alk-O-alk or hydroxyalkyl radical or else R₅₂ and R₅₃ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂,
R₅₄ represents a hydrogen atom or a hydroxyalkyl radical, -alk-COOalk radical, -alk-CONR₅₀R₅₁ radical, -alk-NR₅₀R₅₁ radical, alkoxyalkyl radical, Ar₃ radical, Het₃ radical, —CH₂Ar₃ radical, —CH₂Het₃ radical or alkyl radical optionally substituted with one or more halogen,
R₅₅ represents a hydrogen atom or a hydroxyalkyl radical, -alk-COOalk radical, -alk-CONR₅₀R₅₁ radical, -alk-NR₅₀R₅₁ radical, alkoxyalkyl radical or alkyl radical optionally substituted with one or more halogen,
or else R₅₄ and R₅₅ form, together with the carbon atom to which they are attached, a saturated mono- or bicyclic ring having 3 to 10 ring members optionally comprising another heteroatom chosen from oxygen, sulfur and nitrogen and optionally being substituted by one or more alkyl,
Ar₃ represents a phenyl, naphthyl or indenyl radical, these various radicals optionally being substituted by one or more halogen, alkyl, alkoxy, —CO-alk, cyano, —COOH, —COOalk, —CONR₅₆R₅₇, —CO—NH—NR₅₈R₅₉, alkylsulfanyl, alkylsulfinyl, alkylsulfonyl, -alk-NR₅₈R₅₉, —NR₅₈R₅₉, alkylthioalkyl, formyl, CF₃, OCF₃, Het₃, —O-alk-NH-cycloalkyl, SO₂NH₂, hydroxyl, hydroxyalkyl, —NHCOalk or —NHCOOalk or on 2 adjacent carbon atoms by dioxymethylene,
Het₃ represents a saturated or unsaturated and mono- or bicyclic heterocycle having 3 to 10 ring members and comprising one or more heteroatoms chosen from oxygen, sulfur and nitrogen optionally substituted by one or more alkyl, alkoxy, halogen, alkoxycarbonyl, oxo or hydroxyl, the nitrogenous heterocycles optionally being in their N-oxidized form,
R₅₆ and R₅₇, which are identical or different, represent a hydrogen atom or an alkyl radical or else R₅₆ and R₅₇ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
R₅₈ and R₅₉, which are identical or different, represent a hydrogen atom or an alkyl radical or else R₅₈ and R₅₉ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
alk represents an alkyl or alkylene radical, and
the alkyl, alkylene and alkoxy radicals feature either straight or branched chains and comprise 1 to 6 carbon atoms, the cycloalkyl radicals comprise 3 to 10 carbon atoms and the heterocycloalkyl and heterocyclenyl radicals comprise 3 to 10 carbon atoms, or
an optical isomer of said CB1 antagonist or a pharmaceutically acceptable salt thereof.

5. The method as set forth in claim 4, wherein the CB1 antagonist is selected from the group consisting of:
(RS)-1-[bis(4-chlorophenyl)methyl)]-3-[(3,5-difluoro-phenyl)(methylsulfonyl)methyl]azetidine,
(R)-1-[bis(4-chlorophenyl)methyl)]-3-[(3,5-difluoro-phenyl)(methylsulfonyl)methyl]azetidine,
(S)-1-[bis(4-chlorophenyl)methyl)]-3-[(3,5-difluoro-phenyl)(methylsulfonyl)methyl]azetidine,
(RS)-1-[bis(4-chlorophenyl)methyl)]-3-[(pyrid-3-yl)-(methylsulfonyl)methyl]azetidine,
(R)-1-[bis(4-chlorophenyl)methyl)]-3-[(pyrid-3-yl)-(methylsulfonyl)methyl]azetidine,
(S)-1-[bis(4-chlorophenyl)methyl)]-3-[(pyrid-3-yl)-(methylsulfonyl)methyl]azetidine,
(RS)-1-[bis(3-fluorophenyl)methyl]-3-[(3,5-difluorophenyl)(methylsulfonyl)methyl]azetidine,
(R)-1-[bis(3-fluorophenyl)methyl]-3-[(3,5-difluorophenyl)(methylsulfonyl)methyl]azetidine,
(S)-1-[bis(3-fluorophenyl)methyl]-3-[(3,5-difluorophenyl)(methylsulfonyl)methyl]azetidine,
1-[bis(4-chlorophenyl)methyl]-3-(RS)-{[3-(azetidin-1-yl)phenyl](methylsulfonyl)methyl}azetidine,
1-[bis(4-chlorophenyl)methyl]-3-(R)-{[3-(azetidin-1-yl)phenyl](methylsulfonyl)methyl}azetidine,
1-[bis(4-chlorophenyl)methyl]-3-(S)-{[3-(azetidin-1-yl)phenyl](methylsulfonyl)methyl}azetidine,
(RS)-1-[3-({1-[bis(4-chlorophenyl)methyl]azetidin-3-yl}(methylsulfonyl)methyl)phenyl]pyrrolidine,
(R)-1-[3-({1-[bis(4-chlorophenyl)methyl]azetidin-3-yl}(methylsulfonyl)methyl)phenyl]pyrrolidine,
(S)-1-[3-({1-[bis(4-chlorophenyl)methyl]azetidin-3-yl}(methylsulfonyl)methyl)phenyl]pyrrolidine,
(RS)—N-[3-({1-[bis(4-chlorophenyl)methyl]azetidin-3-yl}(methylsulfonyl)methyl)phenyl]-N-methylamine,
(R)—N-[3-({1-[bis(4-chlorophenyl)methyl]azetidin-3-yl}(methylsulfonyl)methyl)phenyl]-N-methylamine,
(S)—N-[3-({1-[bis(4-chlorophenyl)methyl]azetidin-3-yl}(methylsulfonyl)methyl)phenyl]-N-methylamine,
(RS)-1-[bis(4-chlorophenyl)methyl]-3-[(3,5-bis(trifluoro-methyl)phenyl)(methylsulfonyl)methyl]azetidine,
(R)-1-[bis(4-chlorophenyl)methyl]-3-[(3,5-bis(trifluoro-methyl)phenyl)(methylsulfonyl)methyl]azetidine,
(S)—-[bis(4-chlorophenyl)methyl]-3-[(3,5-bis(trifluoro-methyl)phenyl)(methylsulfonyl)methyl]azetidine,
1-[bis(4-chlorophenyl)methyl]-3-(phenylsulfonyl-methyl)azetidine,
(RS)-1-[bis(4-chlorophenyl)methyl]-3-[(3,5-difluoro-phenyl)(methylsulfonyl)methyl]-3-methylazetidine,
(R)-1-[bis(4-chlorophenyl)methyl]-3-[(3,5-difluorophenyl)methylsulfonylmethyl]-3-methylazetidine,
(S)-1-[bis(4-chlorophenyl)methyl]-3-[(3,5-difluoro-phenyl)(methylsulfonyl)methyl]-3-methylazetidine,
(RS)-2-{1-[bis(4-chlorophenyl)methyl]azetidin-3-yl}-2-(3,5-difluorophenyl)-N-cyclohexylacetamide,
(R)-2-{1-[bis(4-chlorophenyl)methyl]azetidin-3-yl}-2-(3,5-difluorophenyl)-N-cyclohexylacetamide,
(S)-2-{1-[bis(4-chlorophenyl)methyl]azetidin-3-yl}-2-(3,5-difluorophenyl)-N-cyclohexylacetamide,
(RS)-2-{1-[bis(4-chlorophenyl)methyl]azetidin-3-yl}-2-(3,5-difluorophenyl)-N-isobutylacetamide,
(R)-2-{1-[bis(4-chlorophenyl)methyl]azetidin-3-yl}-2-(3,5-difluorophenyl)-N-isobutylacetamide,
(S)-2-{1-[bis(4-chlorophenyl)methyl]azetidin-3-yl}-2-(3,5-difluorophenyl)-N-isobutylacetamide,
(RS)-2-{1-[bis(4-chlorophenyl)methyl]azetidin-3-yl}-2-(3,5-difluorophenyl)-N-cyclopropylmethylacetamide,
(R)-2-{1-[bis(4-chlorophenyl)methyl]azetidin-3-yl}-2-(3,5-difluorophenyl)-N-isopropylacetamide,
(S)-2-{1-[bis(4-chlorophenyl)methyl]azetidin-3-yl}-2-(3,5-difluorophenyl)-N-cyclopropylmethylacetamide,
(RS)-2-{1-[bis(4-chlorophenyl)methyl]azetidin-3-yl}-2-(3,5-difluorophenyl)-N-isopropylacetamide,
(R)-2-{1-[bis(4-chlorophenyl)methyl]azetidin-3-yl}-2-(3,5-difluorophenyl)-N-isopropylacetamide,
(S)-2-{1-[bis(4-chlorophenyl)methyl]azetidin-3-yl}-2-(3,5-difluorophenyl)-N-isopropylacetamide,
(RS)-1-[bis(4-chlorophenyl)methyl]-3-[1-(3,5-difluorophenyl)-1-(methylsulfonyl)ethyl]azetidine,
(R)-1-[bis(4-chlorophenyl)methyl]-3-[1-(3,5-difluorophenyl)-1-(methylsulfonyl)ethyl]azetidine,
(S)-1-[bis(4-chlorophenyl)methyl]-3-[1-(3,5-difluorophenyl)-1-(methylsulfonyl)ethyl]azetidine,
(RS)-1-[bis(4-fluorophenyl)methyl]-3-[(3,5-difluoro-phenyl)(methylsulfonyl)methyl]azetidine,
(R)-1-[bis(4-fluorophenyl)methyl]-3-[(3,5-difluoro-phenyl)(methylsulfonyl)methyl]azetidine,
(S)-1-[bis(4-fluorophenyl)methyl]-3-[(3,5-difluoro-phenyl)(methylsulfonyl)methyl]azetidine,
(RS)-{1-[(3-pyridyl)(4-chlorophenyl)methyl]-3-[(3,5-difluoro-phenyl)(methylsulfonyl)methyl]azetidine,
(SS)-{1-[(3-pyridyl)(4-chlorophenyl)methyl]-3-[(3,5-difluoro-phenyl)(methylsulfonyl)methyl]azetidine,
(RR)-{1-[(3-pyridyl)(4-chlorophenyl)methyl]-3-[(3,5-difluoro-phenyl)(methylsulfonyl)methyl]azetidine,
(SR)-{1-[(3-pyridyl)(4-chlorophenyl)methyl]-3-[(3,5-difluoro-phenyl)(methylsulfonyl)methyl]azetidine,
(RS)-{1-[(4-pyridyl)(4-chlorophenyl)methyl]-3-[(3,5-difluoro-phenyl)(methylsulfonyl)methyl]azetidine,
(SS)-{1-[(4-pyridyl)(4-chlorophenyl)methyl]-3-[(3,5-difluoro-phenyl)(methylsulfonyl)methyl]azetidine,
(RR)-{1-[(4-pyridyl)(4-chlorophenyl)methyl]-3-[(3,5-difluoro-phenyl)(methylsulfonyl)methyl]azetidine,
(SR)-{1-[(4-pyridyl)(4-chlorophenyl)methyl]-3-[(3,5-difluoro-phenyl)(methylsulfonyl)methyl]azetidine,
(RS)-5-((4-chlorophenyl) {3-[(3,5-difluorophenyl)(methylsulfonyl)methyl]azetidin-1-yl}methyl)pyrimidine,
(SR)-5-((4-chlorophenyl) {3-[(3,5-difluorophenyl)(methylsulfonyl)methyl]azetidin-1-yl}methyl)pyrimidine,
(RR)-5-((4-chlorophenyl) {3-[(3,5-difluorophenyl)(methylsulfonyl)methyl]azetidin-1-yl}methyl)pyrimidine,
(SS)-5-((4-chlorophenyl) {3-[(3,5-difluorophenyl)(methylsulfonyl)methyl]azetidin-1-yl}methyl)pyrimidine,
(SS)-{1-[(2-chloropyrid-5-yl)(4-chlorophenyl)methyl]-3-[(3,5-difluorophenyl)(methylsulfonyl)methyl]azetidine,
(RR)-{1-[(2-chloropyrid-5-yl)(4-chlorophenyl)methyl]-3-[(3,5-difluorophenyl)(methylsulfonyl)methyl]azetidine,
(RS)-{1-[(2-chloropyrid-5-yl)(4-chlorophenyl)methyl]-3-[(3,5-difluorophenyl)(methylsulfonyl)methyl]azetidine,
(SR)-{1-[(2-chloropyrid-5-yl)(4-chlorophenyl)methyl]-3-[(3,5-difluorophenyl)(methylsulfonyl)methyl]azetidine,
N-{1-[bis(4-chlorophenyl)methyl]azetidin-3-yl}thien-2-ylsulfonamide,
N-{1-[bis(4-chlorophenyl)methyl]azetidin-3-yl}-4-methoxyphenylsulfonamide,
N-[4-(N-{1-[bis(4-chlorophenyl)methyl]azetidin-3-yl}sulfamoyl)phenyl]acetamide,
N-{1-[bis(4-chlorophenyl)methyl]azetidin-3-yl}-4-methylphenylsulfonamide,
N-{1-[bis(4-chlorophenyl)methyl]azetidin-3-yl}-3,4-dimethoxyphenylsulfonamide,
N-{1-[bis(4-chlorophenyl)methyl]azetidin-3-yl}-3-fluorophenylsulfonamide,
N-{1-[bis(4-chlorophenyl)methyl]azetidin-3-yl}-3,4-dichlorophenylsulfonamide,
N-{1-[bis(4-chlorophenyl)methyl]azetidin-3-yl}-3-cyanophenylsulfonamide,
N-{1-[bis(4-chlorophenyl)methyl]azetidin-3-yl}-2,5-dimethoxyphenylsulfonamide,
N-{1-[bis(4-chlorophenyl)methyl]azetidin-3-yl}-3-trifluoromethylphenylsulfonamide,
N-{1-[bis(4-chlorophenyl)methyl]azetidin-3-yl}naphth-2-ylsulfonamide,
N-{1-[bis(4-chlorophenyl)methyl]azetidin-3-yl}naphth-1-ylsulfonamide,
N-{1-[bis(4-chlorophenyl)methyl]azetidin-3-yl}-3,4-difluorophenylsulfonamide,
N-{1-[bis(4-chlorophenyl)methyl]azetidin-3-yl}-1-methyl-1H-imidazol-4-ylsulfonamide,
N-[4-(N-{1-[bis(4-chlorophenyl)methyl]azetidin-3-yl}sulfamoyl)-2-chlorophenyl]acetamide,
N-{1-[bis(4-chlorophenyl)methyl]azetidin-3-yl}pyrid-3-ylsulfonamide,
N-{1-[bis(4-chlorophenyl)methyl]azetidin-3-yl}-4-fluorophenylsulfonamide,
N-{1-[bis(4-chlorophenyl)methyl]azetidin-3-yl}quinol-8-ylsulfonamide,
N-{1-[bis(4-chlorophenyl)methyl]azetidin-3-yl}phenylsulfonamide,
N-{1-[bis(4-chlorophenyl)methyl]azetidin-3-yl}(phenylmethyl)sulfonamide,
N-{1-[bis(4-chlorophenyl)methyl]azetidin-3-yl}-3,5-difluorophenylsulfonamide,
N-{1-[bis(4-chlorophenyl)methyl]azetidin-3-yl}pyrid-2-ylsulfonamide,
N-{1-[bis(4-chlorophenyl)methyl]azetidin-3-yl}-(3-fluoro-5-pyrrolidin-1-ylphenyl)sulfonamide,
N-{1-[bis(4-chlorophenyl)methyl]azetidin-3-yl}-N-methyl-4-fluorophenylsulfonamide,
N-{1-[bis(4-chlorophenyl)methyl]azetidin-3-yl}-N-methylquinol-8-ylsulfonamide,
N-{1-[bis(4-chlorophenyl)methyl]azetidin-3-yl}-N-methylphenylsulfonamide,
N-{1-[bis(4-chlorophenyl)methyl]azetidin-3-yl}-N-methyl(phenylmethyl)sulfonamide,
N-{1-[bis(4-chlorophenyl)methyl]azetidin-3-yl}-3-sulfamoylphenylsulfonamide,
2-benzenesulfonyl-N-{1-[bis(4-chlorophenyl)methyl]azetidin-3-yl}acetamide,
N-{1-[bis(4-chlorophenyl)methyl]azetidin-3-yl}-2-(toluene-4-sulfonyl)acetamide,
(3-chloro-4-(methylsulfonyl)thiophene-2-carboxy) {1-[bis(4-chlorophenyl)methyl]azetidin-3-yl}amide,
N-{1-[bis(4-chlorophenyl)methyl]azetidin-3-yl}-3-(2-phenylethylenesulfonyl)propionamide,
N-{1-[bis(4-chlorophenyl)methyl]azetidin-3-yl}-4-(methylsulfonyl)benzamide,
(5-(methylsulfonyl)thiophene-2-carboxy)-{1-[bis(4-chlorophenyl)methyl]azetidin-3-yl}amide,
(5-(methylsulfonyl)-3-methyl-4-vinylthiophene-2-carboxy) {1-[bis(4-chlorophenyl)methyl]azetidin-3-yl}amide,
(RS)—N-{1-[{4-chlorophenyl)(pyridin-3-yl)methyl]azetidin-3-yl}-3,5-difluorobenzenesulfonamide,
(RS)—N-{1-[{4-chlorophenyl)(pyrimidin-5-yl)methyl]azetidin-3-yl}-3,5-difluorobenzenesulfonamide,
N-{1-[bis{4-chlorophenyl)methyl]azetidin-3-yl}-N-(6-chloropyrid-2-yl)methylsulfonamide,
N-{1-[bis{4-chlorophenyl)methyl]azetidin-3-yl}-N-(6-ethylpyrid-2-yl)methylsulfonamide,
N-{1-[bis{4-chlorophenyl)methyl]azetidin-3-yl}-N-(quinol-6-yl)methylsulfonamide,
N-{1-[bis{4-chlorophenyl)methyl]azetidin-3-yl}-N-(quinol-5-yl)methylsulfonamide,
N-{1-[bis{4-chlorophenyl)methyl]azetidin-3-yl}-N-(isoquinol-5-yl)methylsulfonamide,
N-{1-[bis{4-chlorophenyl)methyl]azetidin-3-yl}-N-(pyrid-3-yl)methylsulfonamide,
N-{1-[bis{4-chlorophenyl)methyl]azetidin-3-yl}-N-(1-oxidopyrid-3-yl)methylsulfonamide,
N-((1R,2S,4S)bicyclo[2.2.1]hept-2-yl)-N-{1-[bis(4-chlorophenyl)methyl]azetidin-3-yl}methylsulfonamide,
N-((1R,2R,4S)bicyclo[2.2.1]hept-2-yl)-N-{1-[bis(4-chlorophenyl)methyl]azetidin-3-yl}methylsulfonamide,
N-{1-[bis(4-chlorophenyl)methyl]azetidin-3-yl}-N-(3,5-difluorophenyl)methylsulfonamide,
N-{1-[bis(4-chlorophenyl)methyl]azetidin-3-yl}-N-(thiazol-2-yl)methylsulfonamide,
N-{1-[bis(4-chlorophenyl)methyl]azetidin-3-yl}-N-(3-methoxyphenyl)methylsulfonamide,
N-{1-[bis(4-chlorophenyl)methyl]azetidin-3-yl}-N-(3-(hydroxyphenyl)methylsulfonamide,
N-{1-[bis(4-chlorophenyl)methyl]azetidin-3-yl}-N-(3-(hydroxymethyl)phenyl)methylsulfonamide,
ethyl N-{1-[bis(4-chlorophenyl)methyl]azetidin-3-yl}-N-(methylsulfonyl)-3-aminobenzoate
N-{1-[bis(4-chlorophenyl)methyl]azetidin-3-yl}-N-(1-isobutylpiperid-4-yl)methylsulfonamide,
N-benzyl-N-{1-{bis(4-chlorophenyl)methyl]azetidin-3-yl}amine
N-{1-[bis(4-chlorophenyl)methyl]azetidin-3-yl}-N-(3,5-difluorobenzyl)amine,
N-{1-[bis(4-chlorophenyl)methyl]azetidin-3-yl}-N-(3,5-difluorobenzyl)methylsulfonamide,
N-{1-[bis(4-chlorophenyl)methyl]azetidin-3-yl}-N-(pyrid-3-ylmethyl)methylsulfonamide,
N-{1-[bis(4-fluorophenyl)methyl]azetidin-3-yl}-N-(3,5-difluorophenyl)methylsulfonamide,
(RS)—N-{1-[(4-chlorophenyl)(pyrid-3-yl)methyl]azetidin-3-yl}-N-(3,5-difluorophenyl)methylsulfonamide,
(R)—N-{1-[(4-chlorophenyl)(pyrid-3-yl)methyl]azetidin-3-yl}-N-(3,5-difluorophenyl)methylsulfonamide,
(S)—N-{1-[(4-chlorophenyl)(pyrid-3-yl)methyl]azetidin-3-yl}-N-(3,5-difluorophenyl)methylsulfonamide,
(RS)—N-{1-[(4-chlorophenyl)(pyrid-4-yl)methyl]azetidin-3-yl}-N-(3,5-difluorophenyl)methylsulfonamide,
(R)—N-{1-[(4-chlorophenyl)(pyrid-4-yl)methyl]azetidin-3-yl}-N-(3,5-difluorophenyl)methylsulfonamide,
(S)—N-{1-[(4-chlorophenyl)(pyrid-4-yl)methyl]azetidin-3-yl}-N-(3,5-difluorophenyl)methylsulfonamide,
(RS)—N-{1-[(4-chlorophenyl)(pyrimidin-5-yl)methyl]azetidin-3-yl}-N-(3,5-difluorophenyl)methylsulfonamide,
(R)—N-{1-[(4-chlorophenyl)(pyrimidin-5-yl)methyl]azetidin-3-yl}-N-(3,5-difluorophenyl)methylsulfonamide,
(S)—N-{1-[(4-chlorophenyl)(pyrimidin-5-yl)methyl]azetidin-3-yl}-N-(3,5-difluorophenyl)methylsulfonamide, and
N-{1-[bis(4-chlorophenyl)methyl]azetidin-3-yl}-N-(3,5-difluorophenyl)benzylsulfonamide,
or an optical isomer or a pharmaceutically acceptable salt thereof.

6. The method as set forth in claim 4, wherein the CB1 antagonist is selected from the group consisting of:
N-{1-[bis(4-chlorophenyl)methyl]azetidin-3-yl}-N-(pyrid-3-yl)methylsulfonamide, and
N-{1-[bis(4-chlorophenyl)methyl]azetidin-3-yl}-N-(3,5-difluorophenyl)methylsulfonamide
or an optical isomer or a pharmaceutically acceptable salt thereof.

7. The method as set forth in claim 4, wherein said combination improves positive and negative symptoms of schizophrenia.

8. The method as set forth in claim 4, wherein said combination controls weight gain.

9. The method as set forth in claim 4, wherein said combination improves catalepsy.

10. The method as set forth in claim 6, wherein said combination improves positive and negative symptoms of schizophrenia.

11. The method as set forth in claim 6, wherein said combination controls weight gain.

12. The method as set forth in claim 6, wherein said combination improves catalepsy.

13. The method as set forth in claim 4, wherein said antipsychotic agent is selected from the group consisting of: olanzapine, clozapine, haloperidol and haloperidol decanoate, loxapine succinate, molindone hydrochloride, pimozide and risperidone.

14. The method as set forth in claim 6, wherein said antipsychotic agent is selected from the group consisting of: olanzapine, clozapine, haloperidol and haloperidol decanoate, loxapine succinate, molindone hydrochloride, pimozide and risperidone.

15. A pharmaceutical composition comprising one or more CB1 receptor antagonist and one or more antipsychotic agents, in combination with one or more pharmaceutically acceptable carrier, excipient or a diluent, wherein said CB1 antagonist is of formula (I): 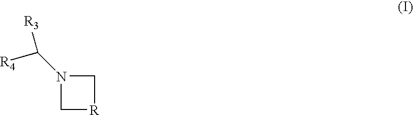
in which
either A:
R represents a CR₁R₂, C═C(R₅)SO₂R₆ or C═C(R₇)SO₂alk radical,
either R₁ represents a hydrogen atom and R₂ represents a —C(R₈)(R₉)(R₁₀), —C(R₈)(R₁₁)(R₁₂), —CO—NR₁₃R₁₄, —CH₂—CO—NR₁₃R₁₄, —CH₂—CO—R₆, —CO—R₆, —CO-cycloalkyl, —SO—R₆, —SO₂—R₆, —C(OH)(R₁₂)(R₆), —C(OH)(R₆)(alkyl), —C(═NOalk)R₆, —C(═NO—CH₂—CH═CH₂)R₆, —CH₂—CH(R₆)NR₃₁R₃₂, —CH₂—C(═NOalk)R₆, —CH(R₆)NR₃₁R₃₂, —CH(R₆)NHSO₂alk, —CH(R₆)NHCONHalk or —CH(R₆)NHCOalk radical,
or R₁ represents an alkyl, NH—R₁₅, cyano, —S-alk-NR₁₆R₁₇, —CH₂—NR₁₈R₁₉ or —NR₂₀R₂₁ radical and R₂ represents a —C(R₈)(R₁₁)(R₁₂) radical,
R₃ and R₄, which are identical or different, represent either an alkyl or cycloalkyl radical, or an aromatic radical chosen from phenyl, naphthyl or indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₂₂R₂₃, —CO—NH—NR₂₄R₂₅, alkylsulfanyl, alkylsulfinyl, alklylsulfonyl, alkylsulfanylalkyl, alkylsulfinylalkyl, alkylsulfonylalkyl, hydroxyalkyl or -alk-NR₂₄R₂₅; or a heteroaromatic radical chosen from the benzofuryl, benzothiazolyl, benzothienyl, benzoxazolyl, chromanyl, 2,3-dihydroxybenzofuryl, 2,3-dihydrobenzothienyl, furyl, imidazolyl, isochromanyl, isoquinolyl, pyrrolyl, pyridyl, pyrimidinyl, quinolyl, 1,2,3,4-tetrahydro-isoquinolyl, thiazolyl and thienyl rings, it being possible for these heteroaromatic radicals to be unsubstituted or substituted by one or more halogen, alkyl, alkoxy, hydroxyl, trifluoromethyl, trifluoromethoxy, cyano, —COOH, —COOalk, —CO—NH—NR₂₄R₂₅, —CONR₂₂R₂₃, -alk-NR₂₄R₂₅, alkylsulfanyl, alkylsulfinyl, alkylsulfonyl, alkylsulfanylalkyl, alkylsulfinylalkyl, alkylsulfonylalkyl or hydroxyalkyl,
R₅ represents a hydrogen atom or an alkyl radical,
R₆ represents an Ar₁ or Het₁ radical,
R₇ represents a cycloalkyl, heterocycloalkyl or heterocyclenyl radical optionally substituted by a —CSO-phenyl radical,
R₈ represents a hydrogen atom or an alkyl radical,
R₉ represents a —CO—NR₂₆R₂₇, —COOH, —COOalk, —CH₂OH, —NH—CO—NH-alk, —CH₂—NHR₂₈ or —NHCOOalk radical,
R₁₀ represents an Ar₁ or Het₁ radical,
R₁₁ represents an —SO₂-alk, —SO₂—Ar₁ or —SO₂-Het₁ radical,
R₁₂ represents a hydrogen atom or an Ar₁ or Het₁ radical,
R₁₃ represents a hydrogen atom or an alkyl radical,
R₁₄ represents an Ar₁, Het₁, -alk-Ar₁ or -alk-Het₁ radical,
R₁₅ represents an alkyl, cycloalkyl or -alk-NR₂₉R₃₀ radical,
R₁₆ and R₁₇, which are identical or different, represent a hydrogen atom or an alkyl radical or else R₁₆ and R₁₇ form, together with the nitrogen atom to which they are attached, a saturated or unsaturated and mono- or bicyclic heterocycle having 3 to 10 ring members and optionally comprising one or more other heteroatoms chosen from oxygen, sulfur and nitrogen and optionally substituted by one or more alkyl radicals,
R₁₈ represents a hydrogen atom or an alkyl radical,
R₁₉ represents a hydrogen atom or an alkyl, cycloalkyl, cycloalkylalkyl, cycloalkylcarbonyl, —SO₂alk, —CO—NHalk or —COOalk radical,
or else, R₁₈ and R₁₉ form, with the nitrogen atom to which they are attached, a saturated or unsaturated and mono- or bicyclic heterocycle having 3 to 10 ring members and optionally comprising one or more heteroatoms chosen from oxygen, sulfur and nitrogen and optionally substituted by one or more alkyl radicals,
—NR₂₀R₂₁ represents a saturated or unsaturated monocyclic heterocycle having 3 to 8 ring members and optionally comprising another heteroatom chosen from oxygen, nitrogen and sulfur,
R₂₂ and R₂₃, which are identical or different, represent a hydrogen atom or an alkyl radical or else R₂₂ and R₂₃ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one more alkyl radicals,
R₂₄ and R₂₅, which are identical or different, represent a hydrogen atom or an alkyl, —COOalk, cycloalkyl, alkylcycloalkyl, -alk-O-alk or hydroxyalkyl radical or else R₂₄ and R₂₅ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂ radicals,
R₂₆ and R₂₇, which are identical or different, represent a hydrogen atom or an alkyl, hydroxyalkyl, cycloalkyl, cycloalkylalkyl, -alk-COOalk, -alk-Ar₁, alk-Het₁, Het₁ or -alk-N(alk)₂ radical, R₂₆ and R₂₇ can also form, with the nitrogen atom to which they are attached, a saturated or unsaturated and mono- or bicyclic heterocycle having 3 to 10 ring members and optionally comprising one or more heteroatoms chosen from oxygen, sulfur and nitrogen and optionally substituted by one or more alkyl, alkoxy or halogen radicals,
R₂₈ represents a —CH₂-alk, benzyl, —SO₂alk, —CONHalk, —COalk, cycloalkylalkylcarbonyl, cycloalkylcarbonyl or —CO—(CH₂)ₙOH radical,
n is equal to 1, 2, or 3,
R₂₉ and R₃₀, which are identical or different, represent a hydrogen atom or an alkyl radical or else R₂₉ and R₃₀ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl radicals,
R₃₁ and R₃₂, which are identical or different, represent a hydrogen atom or an alkyl, Ar₁ or -alk-Ar₁ radical or else R₃₁ and R₃₂ form, together with the nitrogen atom to which they are attached, a heterocycle chosen from aziridinyl, azetidinyl, pyrrolidinyl and piperidinyl,
Ar₁ represents a phenyl or naphthyl radical optionally substituted by one or more substituents chosen from halogen, alkyl, alkoxy, —CO-alk, cyano, —COOH, —COOalk, —CONR₂₂R₂₃, —CO—NH—NR₂₄R₂₅, alkylsulfanyl, alkylsulfinyl, alkylsulfonyl, alkylsulfanylalkyl, alkylsulfinylalkyl, alkylsulfonylalkyl, hydroxyalkyl, -alk-NR₂₄R₂₅, —NR₂₄R₂₅, alkylthioalkyl, formyl, hydroxyl, CF₃, OCF₃, Het₁, O-alk-NH-cycloalkyl or SO₂NH₂,
Het₁ represents a saturated or unsaturated and mono- or bicyclic heterocycle having 3 to 10 ring members and comprising one or more heteroatoms chosen from oxygen, sulfur and nitrogen and optionally substituted by one or more halogen, alkyl, alkoxy, alkoxycarbonyl, —CONR₂₂R₂₃, hydroxyl, hydroxyalkyl, oxo or SO₂NH₂,
or B:
R represents a CHR₃₃ radical,
R₃₃ represents an —NHCOR₃₄ or —N(R₃₅)—Y—R₃₆ radical,
Y is CO or SO₂,
R₃ and R₄, which are identical or different, represent either an aromatic radical chosen from phenyl, naphthyl and indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₃₇R₃₈, —CO—NH—NR₃₉R₄₀, alkylsulfanyl, alkylsulfinyl, alkylsulfonyl, alkylsulfanylalkyl, alkylsulfinylalkyl, alkylsulfonylalkyl, hydroxyalkyl or -alk-NR₃₇R₃₈; or a heteroaromatic radical chosen from the benzofuryl, benzothiazolyl, benzothienyl, benzoxazolyl, chromanyl, 2,3-dihydrobenzofuryl, 2,3-dihydrobenzothienyl, pyrimidinyl, furyl, imidazolyl, isochromanyl, isoquinolyl, pyrrolyl, pyridyl, quinolyl, 1,2,3,4-tetrahydroisoquinolyl, thiazolyl and thienyl rings, it being possible for these heteroaromatic radicals to be unsubstituted or substituted by a halogen, alkyl, alkoxy, hydroxyl, trifluoromethyl, trifluoromethoxy, cyano, —COOH, —COOalk, —CO—NH—NR₃₉R₄₀, —CONR₃₇R₃₈, -alk-NR₃₉R₄₀, alkylsulfanyl, alkylsulfinyl, alkylsulfonyl, alkylsulfanylalkyl, alkylsulfinylalkyl, alkylsulfonylalkyl or hydroxyalkyl,
R₃₄ represents an -alk-SO₂—R₄₁ radical, an -alk-SO₂—CH═CH—R₄₁ radical, a Het₂ radical substituted by —SO₂—R₄₁ or a phenyl radical substituted by —SO₂—R₄₁ or -alk-SO₂-41,
R₃₅ represents a hydrogen atom or an alkyl radical,
R₃₆ represents a phenylalkyl, Het₂ or Ar₂ radical,
R₃₇ and R₃₈, which are identical or different, represent a hydrogen atom or an alkyl radical or else R₃₇ and R₃₈ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
R₃₉ and R₄₀, which are identical or different, represent a hydrogen atom or an alkyl, —COOalk, cycloalkyl, alkylcycloalkyl, -alk-O-alk or hydroxyalkyl radical or else R₃₉ and R₄₀ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂,
R₄₁ represents an alkyl, Ar₂ or Het₂ radical,
Ar₂ represents a phenyl, naphthyl or indenyl radical, these radicals optionally being substituted by one or more halogen, alkyl, alkoxy, cyano, —CO-alk, —COOH, —COOalk, —CONR₄₂R₄₃, —CO—NH—NR₄₄R₄₅, alkylsulfanyl, alkylsulfinyl, alkylsulfonyl, -alk-NR₄₄R₄₅, —NR₄₄R₄₅, alkylthioalkyl, formyl, hydroxyl, hydroxyalkyl, Het₂, —O-alk-NH-cycloalkyl, OCF₃, CF₃, —NH—CO-alk, —SO₂NH₂, —HN—COCH₃, —NH—COOalk or Het₂ or else on two adjacent carbon atoms by a dioxymethylene,
Het₂ represents a saturated or unsaturated and mono- or bicyclic heterocycle having 3 to 10 ring members and comprising one or more heteroatoms chosen from oxygen, sulfur and nitrogen optionally substituted by one or more alkyl, alkoxy, vinyl, halogen, alkoxycarbonyl, oxo, hydroxyl, OCF₃ or CF₃, the nitrogenous heterocycles optionally being in their N-oxidized form,
R₄₂ and R₄₃, which are identical or different, represent a hydrogen atom or an alkyl radical or else R₄₂ and R₄₃ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl radicals,
R₄₄ and R₄₅, which are identical or different, represent a hydrogen atom or an alkyl, —COOalk, cycloalkyl, alkylcycloalkyl, -alk-O-alk or hydroxyalkyl radical or else R₄₄ and R₄₅ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂ radicals,
or C:
R represents a CHR₄₆ radical,
R₄₆ represents an —N(R₄₇)R₄₈, —N(R₄₇)—CO—R₄₈ or —N(R₄₇)—SO₂R₄₉ radical,
R₃ and R₄, which are identical or different, represent either an aromatic radical chosen from phenyl, naphthyl and indenyl, these aromatic radicals being unsubstituted or substituted by one or more halogen, alkyl, alkoxy, formyl, hydroxyl, trifluoromethyl, trifluoromethoxy, —CO-alk, cyano, —COOH, —COOalk, —CONR₅₀R₅₁, —CO—NH—NR₅₂R₅₃, alkylsulfanyl, alkylsulfinyl, alkylsulfonyl, alkylsulfanylalkyl, alkylsulfinylalkyl, alkylsulfonylalkyl, hydroxyalkyl or -alk-NR₇R₈ radicals; or a heteroaromatic radical chosen from the benzofuryl, benzothiazolyl, benzothienyl, benzoxazolyl, chromanyl, 2,3-dihydrobenzofuryl, 2,3-dihydrobenzothienyl, furyl, imidazolyl, isochromanyl, isoquinolyl, pyrrolyl, pyridyl, pyrimidyl, quinolyl, 1,2,3,4-tetrahydroisoquinolyl, thiazolyl and thienyl rings, it being possible for these heteroaromatic radicals to be unsubstituted or substituted by a halogen, alkyl, alkoxy, hydroxyl, trifluoromethyl, trifluoromethoxy, cyano, —COOH, —COOalk, —CO—NH—NR₅₂R₅₃, —CONR₅₀R₅₁, -alk-NR₅₂R₅₃, alkylsulfanyl, alkylsulfinyl, alkylsulfonyl, alkylsulfanylalkyl, alkylsulfinylalkyl, alkylsulfonylalkyl or hydroxyalkyl radical,
R₄₇ represents a —C(R₅₄)(R₅₅)-Het₃, -Het₃, —C(R₅₄)(R₅₅)—Ar₃, Ar₃, cycloalkyl or norbornyl radical,
R₄₈ represents a hydrogen atom or a hydroxyalkyl radical, -alk-COOalk radical, -alk-CONR₅₀R₅₁ radical, -alk-NR₅₀R₅₁ radical, alkoxy radical, Ar₃ radical, Het₃ radical, —CH₂Ar₃ radical, —CH₂Het₃ radical or alkyl radical optionally substituted with one or more halogen,
R₄₉ represents a hydroxyalkyl radical, -alk-COOalk radical, -alk-CONR₅₀R₅₁ radical, -alk-NR₅₀R₅₁ radical, alkoxy radical, Ar₃ radical, Het₃ radical, —CH₂Ar₃ radical, —CH₂Het₃ radical or alkyl radical optionally substituted with one or more halogen,
R₅₀ and R₅₁, which are identical or different, represent a hydrogen atom or an alkyl radical or else R₅₀ and R₅₁ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
R₅₂ and R₅₃, which are identical or different, represent a hydrogen atom or an alkyl, —COOalk, cycloalkyl, alkylcycloalkyl, -alk-O-alk or hydroxyalkyl radical or else R₅₂ and R₅₃ form, together with the nitrogen atom to which they are attached, a saturated or unsaturated and mono- or bicyclic heterocycle having 3 to 10 ring members optionally comprising another heteroatom chosen from oxygen, sulfur and nitrogen and optionally being substituted by one or more alkyl, —COalk, —COOalk, —CO—NHalk, —CS—NHalk, oxo, hydroxyalkyl, -alk-O-alk or —CO—NH₂,
R₅₄ represents a hydrogen atom or a hydroxyalkyl radical, -alk-COOalk radical, -alk-CONR₅₀R₅₁ radical, -alk-NR₅₀R₅₁ radical, alkoxyalkyl radical, Ar₃ radical, Het₃ radical, —CH₂Ar₃ radical, —CH₂Het₃ radical or alkyl radical optionally substituted with one or more halogen,
R₅₅ represents a hydrogen atom or a hydroxyalkyl radical, -alk-COOalk radical, -alk-CONR₅₀R₅₁ radical, -alk-NR₅₀R₅₁ radical, alkoxyalkyl radical or alkyl radical optionally substituted with one or more halogen,
or else R₅₄ and R₅₅ form, together with the carbon atom to which they are attached, a saturated mono- or bicyclic ring having 3 to 10 ring members optionally comprising another heteroatom chosen from oxygen, sulfur and nitrogen and optionally being substituted by one or more alkyl,
Ar₃ represents a phenyl, naphthyl or indenyl radical, these various radicals optionally being substituted by one or more halogen, alkyl, alkoxy, —CO-alk, cyano, —COOH, —COOalk, —CONR₅₆R₅₇, —CO—NH—NR₅₈R₅₉, alkylsulfanyl, alkylsulfinyl, alkylsulfonyl, -alk-NR₅₈R₅₉, —NR₅₈R₅₉, alkylthioalkyl, formyl, CF₃, OCF₃, Het₃, —O-alk-NH-cycloalkyl, SO₂NH₂, hydroxyl, hydroxyalkyl, —NHCOalk or —NHCOOalk or on 2 adjacent carbon atoms by dioxymethylene,
Het₃ represents a saturated or unsaturated and mono- or bicyclic heterocycle having 3 to 10 ring members and comprising one or more heteroatoms chosen from oxygen, sulfur and nitrogen optionally substituted by one or more alkyl, alkoxy, halogen, alkoxycarbonyl, oxo or hydroxyl, the nitrogenous heterocycles optionally being in their N-oxidized form,
R₅₆ and R₅₇, which are identical or different, represent a hydrogen atom or an alkyl radical or else R₅₆ and R₅₇ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
R₅₈ and R₅₉, which are identical or different, represent a hydrogen atom or an alkyl radical or else R₅₈ and R₅₉ form, together with the nitrogen atom to which they are attached, a saturated mono- or bicyclic heterocycle having 3 to 10 ring members optionally comprising another heteroatom chosen from oxygen, sulfur and nitrogen and optionally being substituted by one or more alkyl,
alk represents an alkyl or alkylene radical, and
the alkyl, alkylene and alkoxy radicals feature either straight or branched chains and comprise 1 to 6 carbon atoms, the cycloalkyl radicals comprise 3 to 10 carbon atoms and the heterocycloalkyl and heterocyclenyl radicals comprise 3 to 10 carbon atoms, or
an optical isomer of said CB1 antagonist or a pharmaceutically acceptable salt thereof.

16. The composition as set forth in claim 15, wherein the CB1 antagonist is selected from the group consisting of:
(RS)-1-[bis(4-chlorophenyl)methyl)]-3-[(3,5-difluoro-phenyl)(methylsulfonyl)methyl]azetidine,
(R)-1-[bis(4-chlorophenyl)methyl)]-3-[(3,5-difluoro-phenyl)(methylsulfonyl)methyl]azetidine,
(S)-1-[bis(4-chlorophenyl)methyl)]-3-[(3,5-difluoro-phenyl)(methylsulfonyl)methyl]azetidine,
(RS)-1-[bis(4-chlorophenyl)methyl)]-3-[(pyrid-3-yl)-(methylsulfonyl)methyl]azetidine,
(R)-1-[bis(4-chlorophenyl)methyl)]-3-[(pyrid-3-yl)-(methylsulfonyl)methyl]azetidine,
(S)-1-[bis(4-chlorophenyl)methyl)]-3-[(pyrid-3-yl)-(methylsulfonyl)methyl]azetidine,
(RS)-1-[bis(3-fluorophenyl)methyl]-3-[(3,5-difluorophenyl)(methylsulfonyl)methyl]azetidine,
(R)-1-[bis(3-fluorophenyl)methyl]-3-[(3,5-difluorophenyl)(methylsulfonyl)methyl]azetidine,
(S)-1-[bis(3-fluorophenyl)methyl]-3-[(3,5-difluorophenyl)(methylsulfonyl)methyl]azetidine,
1-[bis(4-chlorophenyl)methyl]-3-(RS)-{[3-(azetidin-1-yl)phenyl](methylsulfonyl)methyl}azetidine,
1-[bis(4-chlorophenyl)methyl]-3-(R)-{[3-(azetidin-1-yl)phenyl](methylsulfonyl)methyl}azetidine,
1-[bis(4-chlorophenyl)methyl]-3-(S)-{[3-(azetidin-1-yl)phenyl](methylsulfonyl)methyl}azetidine,
(RS)-1-[3-({1-[bis(4-chlorophenyl)methyl]azetidin-3-yl}(methylsulfonyl)methyl)phenyl]pyrrolidine,
(R)-1-[3-({1-[bis(4-chlorophenyl)methyl]azetidin-3-yl}(methylsulfonyl)methyl)phenyl]pyrrolidine,
(S)-1-[3-({1-[bis(4-chlorophenyl)methyl]azetidin-3-yl}(methylsulfonyl)methyl)phenyl]pyrrolidine,
(RS)—N-[3-({1-[bis(4-chlorophenyl)methyl]azetidin-3-yl}(methylsulfonyl)methyl)phenyl]-N-methylamine,
(R)—N-[3-({1-[bis(4-chlorophenyl)methyl]azetidin-3-yl}(methylsulfonyl)methyl)phenyl]-N-methylamine,
(S)—N-[3-({1-[bis(4-chlorophenyl)methyl]azetidin-3-yl}(methylsulfonyl)methyl)phenyl]-N-methylamine,
(RS)-1-[bis(4-chlorophenyl)methyl]-3-[(3,5-bis(trifluoro-methyl)phenyl)(methylsulfonyl)methyl]azetidine,
(R)-1-[bis(4-chlorophenyl)methyl]-3-[(3,5-bis(trifluoro-methyl)phenyl)(methylsulfonyl)methyl]azetidine,
(S)-1-[bis(4-chlorophenyl)methyl]-3-[(3,5-bis(trifluoro-methyl)phenyl)(methylsulfonyl)methyl]azetidine,
1-[bis(4-chlorophenyl)methyl]-3-(phenylsulfonyl-methyl)azetidine,
(RS)-1-[bis(4-chlorophenyl)methyl]-3-[(3,5-difluoro-phenyl)(methylsulfonyl)methyl]-3-methylazetidine,
(R)-1-[bis(4-chlorophenyl)methyl]-3-[(3,5-difluorophenyl)methylsulfonylmethyl]-3-methylazetidine,
(S)-1-[bis(4-chlorophenyl)methyl]-3-[(3,5-difluoro-phenyl)(methylsulfonyl)methyl]-3-methylazetidine,
(RS)-2-{1-[bis(4-chlorophenyl)methyl]azetidin-3-yl}-2-(3,5-difluorophenyl)-N-cyclohexylacetamide,
(R)-2-{1-[bis(4-chlorophenyl)methyl]azetidin-3-yl}-2-(3,5-difluorophenyl)-N-cyclohexylacetamide,
(S)-2-{1-[bis(4-chlorophenyl)methyl]azetidin-3-yl}-2-(3,5-difluorophenyl)-N-cyclohexylacetamide,
(RS)-2-{1-[bis(4-chlorophenyl)methyl]azetidin-3-yl}-2-(3,5-difluorophenyl)-N-isobutylacetamide,
(R)-2-{1-[bis(4-chlorophenyl)methyl]azetidin-3-yl}-2-(3,5-difluorophenyl)-N-isobutylacetamide,
(S)-2-{1-[bis(4-chlorophenyl)methyl]azetidin-3-yl}-2-(3,5-difluorophenyl)-N-isobutylacetamide,
(RS)-2-{1-[bis(4-chlorophenyl)methyl]azetidin-3-yl}-2-(3,5-difluorophenyl)-N-cyclopropylmethylacetamide,
(R)-2-{1-[bis(4-chlorophenyl)methyl]azetidin-3-yl}-2-(3,5-difluorophenyl)-N-isopropylacetamide,
(S)-2-{1-[bis(4-chlorophenyl)methyl]azetidin-3-yl}-2-(3,5-difluorophenyl)-N-cyclopropylmethylacetamide,
(RS)-2-{1-[bis(4-chlorophenyl)methyl]azetidin-3-yl}-2-(3,5-difluorophenyl)-N-isopropylacetamide,
(R)-2-{1-[bis(4-chlorophenyl)methyl]azetidin-3-yl}-2-(3,5-difluorophenyl)-N-isopropylacetamide,
(S)-2-{1-[bis(4-chlorophenyl)methyl]azetidin-3-yl}-2-(3,5-difluorophenyl)-N-isopropylacetamide,
(RS)-1-[bis(4-chlorophenyl)methyl]-3-[1-(3,5-difluorophenyl)-1-(methylsulfonyl)ethyl]azetidine,
(R)-1-[bis(4-chlorophenyl)methyl]-3-[1-(3,5-difluorophenyl)-1-(methylsulfonyl)ethyl]azetidine,
(S)-1-[bis(4-chlorophenyl)methyl]-3-[1-(3,5-difluorophenyl)-1-(methylsulfonyl)ethyl]azetidine,
(RS)-1-[bis(4-fluorophenyl)methyl]-3-[(3,5-difluoro-phenyl)(methylsulfonyl)methyl]azetidine,
(R)-1-[bis(4-fluorophenyl)methyl]-3-[(3,5-difluoro-phenyl)(methylsulfonyl)methyl]azetidine,
(S)-1-[bis(4-fluorophenyl)methyl]-3-[(3,5-difluoro-phenyl)(methylsulfonyl)methyl]azetidine,
(RS)-{1-[(3-pyridyl)(4-chlorophenyl)methyl]-3-[(3,5-difluoro-phenyl)(methylsulfonyl)methyl]azetidine,
(SS)-{1-[(3-pyridyl)(4-chlorophenyl)methyl]-3-[(3,5-difluoro-phenyl)(methylsulfonyl)methyl]azetidine,
(RR)-{1-[(3-pyridyl)(4-chlorophenyl)methyl]-3-[(3,5-difluoro-phenyl)(methylsulfonyl)methyl]azetidine,
(SR)-{1-[(3-pyridyl)(4-chlorophenyl)methyl]-3-[(3,5-difluoro-phenyl)(methylsulfonyl)methyl]azetidine,
(RS)-{1-[(4-pyridyl)(4-chlorophenyl)methyl]-3-[(3,5-difluoro-phenyl)(methylsulfonyl)methyl]azetidine,
(SS)-{1-[(4-pyridyl)(4-chlorophenyl)methyl]-3-[(3,5-difluoro-phenyl)(methylsulfonyl)methyl]azetidine,
(RR)-{1-[(4-pyridyl)(4-chlorophenyl)methyl]-3-[(3,5-difluoro-phenyl)(methylsulfonyl)methyl]azetidine,
(SR)-{1-[(4-pyridyl)(4-chlorophenyl)methyl]-3-[(3,5-difluoro-phenyl)(methylsulfonyl)methyl]azetidine,
(RS)-5-((4-chlorophenyl) {3-[(3,5-difluorophenyl)(methylsulfonyl)methyl]azetidin-1-yl}methyl)pyrimidine,
(SR)-5-((4-chlorophenyl) {3-[(3,5-difluorophenyl)(methylsulfonyl)methyl]azetidin-1-yl}methyl)pyrimidine,
(RR)-5-((4-chlorophenyl) {3-[(3,5-difluorophenyl)(methylsulfonyl)methyl]azetidin-1-yl}methyl)pyrimidine,
(SS)-5-((4-chlorophenyl) {3-[(3,5-difluorophenyl)(methylsulfonyl)methyl]azetidin-1-yl}methyl)pyrimidine,
(SS)-{1-[(2-chloropyrid-5-yl)(4-chlorophenyl)methyl]-3-[(3,5-difluorophenyl)(methylsulfonyl)methyl]azetidine,
(RR)-{1-[(2-chloropyrid-5-yl)(4-chlorophenyl)methyl]-3-[(3,5-difluorophenyl)(methylsulfonyl)methyl]azetidine,
(RS)-{1-[(2-chloropyrid-5-yl)(4-chlorophenyl)methyl]-3-[(3,5-difluorophenyl)(methylsulfonyl)methyl]azetidine,
(SR)-{1-[(2-chloropyrid-5-yl)(4-chlorophenyl)methyl]-3-[(3,5-difluorophenyl)(methylsulfonyl)methyl]azetidine,
N-{1-[bis(4-chlorophenyl)methyl]azetidin-3-yl}thien-2-ylsulfonamide,
N-{1-[bis(4-chlorophenyl)methyl]azetidin-3-yl}-4-methoxyphenylsulfonamide,
N-[4-(N-{1-[bis(4-chlorophenyl)methyl]azetidin-3-yl}sulfamoyl)phenyl]acetamide,
N-{1-[bis(4-chlorophenyl)methyl]azetidin-3-yl}-4-methylphenylsulfonamide,
N-{1-[bis(4-chlorophenyl)methyl]azetidin-3-yl}-3,4-dimethoxyphenylsulfonamide,
N-{1-[bis(4-chlorophenyl)methyl]azetidin-3-yl}-3-fluorophenylsulfonamide,
N-{1-[bis(4-chlorophenyl)methyl]azetidin-3-yl}-3,4-dichlorophenylsulfonamide,
N-{1-[bis(4-chlorophenyl)methyl]azetidin-3-yl}-3-cyanophenylsulfonamide,
N-{1-[bis(4-chlorophenyl)methyl]azetidin-3-yl}-2,5-dimethoxyphenylsulfonamide,
N-{1-[bis(4-chlorophenyl)methyl]azetidin-3-yl}-3-trifluoromethylphenylsulfonamide,
N-{1-[bis(4-chlorophenyl)methyl]azetidin-3-yl}naphth-2-ylsulfonamide,
N-{1-[bis(4-chlorophenyl)methyl]azetidin-3-yl}naphth-1-ylsulfonamide,
N-{1-[bis(4-chlorophenyl)methyl]azetidin-3-yl}-3,4-difluorophenylsulfonamide,
N-{1-[bis(4-chlorophenyl)methyl]azetidin-3-yl}-1-methyl-1H-imidazol-4-ylsulfonamide,
N-[4-(N-{1-[bis(4-chlorophenyl)methyl]azetidin-3-yl}sulfamoyl)-2-chlorophenyl]acetamide,
N-{1-[bis(4-chlorophenyl)methyl]azetidin-3-yl}pyrid-3-ylsulfonamide,
N-{1-[bis(4-chlorophenyl)methyl]azetidin-3-yl}-4-fluorophenylsulfonamide,
N-{1-[bis(4-chlorophenyl)methyl]azetidin-3-yl}quinol-8-ylsulfonamide,
N-{1-[bis(4-chlorophenyl)methyl]azetidin-3-yl}phenylsulfonamide,
N-{1-[bis(4-chlorophenyl)methyl]azetidin-3-yl}(phenylmethyl)sulfonamide,
N-{1-[bis(4-chlorophenyl)methyl]azetidin-3-yl}-3,5-difluorophenylsulfonamide,
N-{1-[bis(4-chlorophenyl)methyl]azetidin-3-yl}pyrid-2-ylsulfonamide,
N-{1-[bis(4-chlorophenyl)methyl]azetidin-3-yl}-(3-fluoro-5-pyrrolidin-1-ylphenyl)sulfonamide,
N-{1-[bis(4-chlorophenyl)methyl]azetidin-3-yl}-N-methyl-4-fluorophenylsulfonamide,
N-{1-[bis(4-chlorophenyl)methyl]azetidin-3-yl}-N-methylquinol-8-ylsulfonamide,
N-{1-[bis(4-chlorophenyl)methyl]azetidin-3-yl}-N-methylphenylsulfonamide,
N-{1-[bis(4-chlorophenyl)methyl]azetidin-3-yl}-N-methyl(phenylmethyl)sulfonamide,
N-{1-[bis(4-chlorophenyl)methyl]azetidin-3-yl}-3-sulfamoylphenylsulfonamide,
2-benzenesulfonyl-N-{1-[bis(4-chlorophenyl)methyl]azetidin-3-yl}acetamide,
N-{1-[bis(4-chlorophenyl)methyl]azetidin-3-yl}-2-(toluene-4-sulfonyl)acetamide,
(3-chloro-4-(methylsulfonyl)thiophene-2-carboxy) {1-[bis(4-chlorophenyl)methyl]azetidin-3-yl}amide,
N-{1-[bis(4-chlorophenyl)methyl]azetidin-3-yl}-3-(2-phenylethylenesulfonyl)propionamide,
N-{1-[bis(4-chlorophenyl)methyl]azetidin-3-yl}-4-(methylsulfonyl)benzamide,
(5-(methylsulfonyl)thiophene-2-carboxy)-{1-[bis(4-chlorophenyl)methyl]azetidin-3-yl}amide,
(5-(methylsulfonyl)-3-methyl-4-vinylthiophene-2-carboxy) {1-[bis(4-chlorophenyl)methyl]azetidin-3-yl}amide,
(RS)—N-{1-[{4-chlorophenyl)(pyridin-3-yl)methyl]azetidin-3-yl}-3,5-difluorobenzenesulfonamide,
(RS)—N-{1-[{4-chlorophenyl)(pyrimidin-5-yl)methyl]azetidin-3-yl}-3,5-difluorobenzenesulfonamide,
N-{1-[bis{4-chlorophenyl)methyl]azetidin-3-yl}-N-(6-chloropyrid-2-yl)methylsulfonamide,
N-{1-[bis{4-chlorophenyl)methyl]azetidin-3-yl}-N-(6-ethylpyrid-2-yl)methylsulfonamide,
N-{1-[bis{4-chlorophenyl)methyl]azetidin-3-yl}-N-(quinol-6-yl)methylsulfonamide,
N-{1-[bis{4-chlorophenyl)methyl]azetidin-3-yl}-N-(quinol-5-yl)methylsulfonamide,
N-{1-[bis{4-chlorophenyl)methyl]azetidin-3-yl}-N-(isoquinol-5-yl)methylsulfonamide,
N-{1-[bis{4-chlorophenyl)methyl]azetidin-3-yl}-N-(pyrid-3-yl)methylsulfonamide,
N-{1-[bis{4-chlorophenyl)methyl]azetidin-3-yl}-N-(1-oxidopyrid-3-yl)methylsulfonamide,
N-((1R,2S,4S)bicyclo[2.2.1]hept-2-yl)-N-{1-[bis(4-chlorophenyl)methyl]azetidin-3-yl}methylsulfonamide,
N-((1R,2R,4S)bicyclo[2.2.1]hept-2-yl)-N-{1-[bis(4-chlorophenyl)methyl]azetidin-3-yl}methylsulfonamide,
N-{1-[bis(4-chlorophenyl)methyl]azetidin-3-yl}-N-(3,5-difluorophenyl)methylsulfonamide,
N-{1-[bis(4-chlorophenyl)methyl]azetidin-3-yl}-N-(thiazol-2-yl)methylsulfonamide,
N-{1-[bis(4-chlorophenyl)methyl]azetidin-3-yl}-N-(3-methoxyphenyl)methylsulfonamide,
N-{1-[bis(4-chlorophenyl)methyl]azetidin-3-yl}-N-(3-(hydroxyphenyl)methylsulfonamide,
N-{1-[bis(4-chlorophenyl)methyl]azetidin-3-yl}-N-(3-(hydroxymethyl)phenyl)methylsulfonamide,
ethyl N-{1-[bis(4-chlorophenyl)methyl]azetidin-3-yl}-N-(methylsulfonyl)-3-aminobenzoate
N-{1-[bis(4-chlorophenyl)methyl]azetidin-3-yl}-N-(1-isobutylpiperid-4-yl)methylsulfonamide,
N-benzyl-N-{1-{bis(4-chlorophenyl)methyl]azetidin-3-yl}amine
N-{1-[bis(4-chlorophenyl)methyl]azetidin-3-yl}-N-(3,5-difluorobenzyl)amine,
N-{1-[bis(4-chlorophenyl)methyl]azetidin-3-yl}-N-(3,5-difluorobenzyl)methylsulfonamide,
N-{1-[bis(4-chlorophenyl)methyl]azetidin-3-yl}-N-(pyrid-3-ylmethyl)methylsulfonamide,
N-{1-[bis(4-fluorophenyl)methyl]azetidin-3-yl}-N-(3,5-difluorophenyl)methylsulfonamide,
(RS)—N-{1-[(4-chlorophenyl)(pyrid-3-yl)methyl]azetidin-3-yl}-N-(3,5-difluorophenyl)methylsulfonamide,
(R)—N-{1-[(4-chlorophenyl)(pyrid-3-yl)methyl]azetidin-3-yl}-N-(3,5-difluorophenyl)methylsulfonamide,
(S)—N-{1-[(4-chlorophenyl)(pyrid-3-yl)methyl]azetidin-3-yl}-N-(3,5-difluorophenyl)methylsulfonamide,
(RS)—N-{1-[(4-chlorophenyl)(pyrid-4-yl)methyl]azetidin-3-yl}-N-(3,5-difluorophenyl)methylsulfonamide,
(R)—N-{1-[(4-chlorophenyl)(pyrid-4-yl)methyl]azetidin-3-yl}-N-(3,5-difluorophenyl)methylsulfonamide,
(S)—N-{1-[(4-chlorophenyl)(pyrid-4-yl)methyl]azetidin-3-yl}-N-(3,5-difluorophenyl)methylsulfonamide,
(RS)—N-{1-[(4-chlorophenyl)(pyrimidin-5-yl)methyl]azetidin-3-yl}-N-(3,5-difluorophenyl)methylsulfonamide,
(R)—N-{1-[(4-chlorophenyl)(pyrimidin-5-yl)methyl]azetidin-3-yl}-N-(3,5-difluorophenyl)methylsulfonamide,
(S)—N-{1-[(4-chlorophenyl)(pyrimidin-5-yl)methyl]azetidin-3-yl}-N-(3,5-difluorophenyl)methylsulfonamide, and
N-{1-[bis(4-chlorophenyl)methyl]azetidin-3-yl}-N-(3,5-difluorophenyl)benzylsulfonamide,
or an optical isomer or a pharmaceutically acceptable salt thereof.

17. The composition as set forth in claim 15, wherein the CB1 antagonist is selected from the group consisting of:
N-{1-[bis(4-chlorophenyl)methyl]azetidin-3-yl}-N-(pyrid-3-yl)methylsulfonamide, and
N-{1-[bis(4-chlorophenyl)methyl]azetidin-3-yl}-N-(3,5-difluorophenyl)methylsulfonamide
or an optical isomer or a pharmaceutically acceptable salt thereof.

18. The composition as set forth in claim 15, wherein said antipsychotic agent is selected from the group consisting of: olanzapine, clozapine, haloperidol and haloperidol decanoate, loxapine succinate, molindone hydrochloride, pimozide and risperidone.
